(19) United States
(12) Patent Application Publication
(10) Pub. No.: US 2024/0083938 A1
(43) Pub. Date: Mar. 14, 2024

(54) Peptide Synthesis Processes

(71) Applicant: CEM Corporation, Matthews, NC (US)

(72) Inventors: Jonathan McKinnon Collins, Charlotte, NC (US); Sandeep K. Singh, Charlotte, NC (US)

(21) Appl. No.: 18/237,576

(22) Filed: Aug. 24, 2023

Related U.S. Application Data
(60) Provisional application No. 63/401,349, filed on Aug. 26, 2022.
(60) Provisional application No. 63/442,216, filed on Jan. 31, 2023.
(60) Provisional application No. 63/452,550, filed on Mar. 16, 2023.
(60) Provisional application No. 63/452,674, filed on Mar. 16, 2023.
(60) Provisional application No. 63/521,623, filed on Jun. 16, 2023.
(60) Provisional application No. 63/532,041, filed on Aug. 10, 2023.

(51) Int. Cl. C07K 1/04; C07K 1/10

(52) U.S. Cl. CPC C07K 1/04; C07K 1/10

(57) ABSTRACT

A process for deprotecting a protected amino acid during solid phase peptide synthesis (SPPS) includes removing a protecting group of a protected amino acid in a reaction vessel with a deprotecting base present in the reaction vessel in an amount greater than zero to about 5 vol %, based on the total volume (100 vol %) of a deprotection reaction mixture in the reaction vessel. At least a portion of the deprotecting base evaporates into an upper interior portion of the reaction vessel (e.g., the headspace) during the removing step. The process also includes directing an inert gas through the reaction vessel to remove evaporated deprotecting base from the interior of the reaction vessel (e.g., from the headspace) during the step of removing the protecting group.

### CROSS-REFERENCE TO PRIORITY APPLICATIONS

This application hereby claims the benefit of pending U.S. Patent Application No. 63/401,349, filed Aug. 26, 2022; pending U.S. Patent Application No. 63/442,216, filed Jan. 31, 2023; pending U.S. Patent Application No. 63/452,550, filed Mar. 16, 2023; pending U.S. Patent Application No. 63/452,674, filed Mar. 16, 2023; pending U.S. Patent Application No. 63/521,623, filed Jun. 16, 2023; and pending U.S. Patent Application No. 63/532,041, filed Aug. 10, 2023, the entire disclosure of each of which is hereby incorporated by reference in their entirety.

### FIELD

The present disclosure relates to processes for deprotecting a protected amino acid and/or a protected peptide (e.g., as a step in peptide synthesis including deprotecting and coupling steps, such as solid phase peptide synthesis, also referred to herein as SPPS, and/or liquid and/or solution phase peptide synthesis, also referred to herein as LPPS).

### BACKGROUND

Since its inception in 1963, solid phase peptide synthesis (SPPS) has been a major enabling tool for peptide synthesis. SPPS dramatically simplified the production of peptides compared to liquid phase peptide synthesis (LPPS) by allowing straightforward isolation of products by simple filtration at each step as opposed to more tedious extraction processes after each deprotection and coupling step. However, compared to LPPS, SPPS results in significant waste production from successive washing steps between each deprotection and coupling step. Historically, about 5 washes were needed between each step resulting in 80-90% of the total waste being generated from washing.

The use of microwave energy and heating in general was initially applied to SPPS for accelerating synthesis times and improving purity by driving reaction steps toward completion. While successful in this regard, these initial efforts did not fundamentally eliminate the need for washing in the process. Later developments introduced a microwave-assisted high efficiency SPPS process for 9-fluorenylmethyloxycarbonyl (Fmoc) SPPS that eliminated washing after each coupling step. This was based on the insight that residual activated amino ester is quickly scavenged by the deprotection base before an insertion could occur. This approach eliminated half of the washing and reduced the overall cycle time of the process. It was later further demonstrated that deprotection base could be added directly to the post-coupling solution without any draining. This “one-pot” deprotection and coupling process allows for reuse of both solvent and heat from the coupling solution to facilitate the deprotection process resulting in further solvent, energy, and time savings.

Despite the foregoing, SPPS processes still generally requires significant washing during each amino acid addition cycle, primarily due to the need to remove deprotection reagents that cause undesirable insertion and deletion impurities to form during the next coupling step. Typically, if residual base from deprotection contaminates the next coupling step, it will remove the Fmoc protecting group on the next amino acid leading to the undesirable insertion of an additional amino acid onto the growing chain. Furthermore, residual base can react with and consume activated amino ester before it reacts with the peptide terminus. The result is the generation of both insertions and deletions of the next amino acid, which can lead to impurities that are difficult to separate (e.g., by reverse-phase HPLC). Thus, washing after the deprotection step has been considered unavoidable.

### SUMMARY

The present disclosure relates to processes for deprotecting a protected amino acid and/or a protected peptide (e.g., as a step in peptide synthesis including deprotecting and coupling steps, such as solid phase peptide synthesis, also referred to herein as SPPS, and/or liquid and/or solution phase peptide synthesis, also referred to herein as LPPS). The deprotection reaction removes a protecting group of the protected amino acid and/or a protected peptide (deprotects the amino acid and/or deprotects the peptide) to prepare the amino acid and/or peptide for a coupling reaction with a second amino acid.

The terms “liquid phase peptide synthesis” and “solution phase peptide synthesis” may be used interchangeably herein and/or may be generally referred to herein as “LPPS.”

In some embodiments, in contrast to prior deprotection steps of a SPPS process, the present disclosure can help reduce the amount of solvent used for washing step(s) after deprotection or can help eliminate washing step(s) after deprotection. This in turn can, in some embodiments, reduce or eliminate washing step(s) (e.g., reduce or eliminate all post-coupling washing steps and reduce or eliminate all post deprotection washing steps) to provide significant reduction in waste (e.g., up to 95% reduction in overall waste) and associated cost and time savings.

In some embodiments, in contrast to prior deprotection steps of a LPPS process, the present disclosure can help reduce the amount of solvent used for extraction step(s) after deprotection and before coupling. This can, in some embodiments, reduce the amount of solvent used in post-deprotection extraction step(s) and/or reduce or eliminate washing step(s) post-coupling to provide significant reduction in waste and associated cost and time savings.

The process for deprotecting a protected amino acid (e.g., during solid phase peptide synthesis and/or during liquid phase peptide synthesis) includes a step of removing a protecting group of a protected amino acid and/or a protected peptide in a reaction vessel (e.g., in a batch-type SPPS and/or LPPS reaction vessel) using a deprotecting base in an amount of about 5 vol % or less, based on the total volume (100 vol %) of a deprotection reaction mixture (e.g., a deprotection reaction solution) in the reaction vessel.

In some embodiments, the deprotecting base may be present in the reaction vessel in an amount from about 1 vol % to about 5 vol %, for example from about 2 vol % to about 5 vol %, for example from about 2 vol % to about 4.5 vol %, for example from about 3 vol % to about 4.5 vol %, and as another example from about 3.5 vol % to about 4.5 vol %, based on the total volume (100 vol %) of a deprotection reaction mixture (e.g., a deprotection reaction solution) in the reaction vessel. In some embodiments, the deprotecting base may be present in the reaction vessel in an amount greater than zero to about 4.5 vol %, for example about 2 vol % to about 4.5 vol %, based on the total volume of the deprotection reaction mixture (e.g., the deprotection reaction solution). In some embodiments, the deprotecting base may be present in the reaction vessel in an amount greater than zero to about 4 vol %, for example about 2 vol % to about 4 vol %, based on the total volume of the deprotection reaction mixture (e.g., the deprotection reaction solution). In some embodiments, the deprotecting base may be present in the reaction vessel in an amount greater than zero to about 3.5 vol %, for example about 2 vol % to about 3.5 vol %, based on the total volume of the deprotection reaction mixture (e.g., the deprotection reaction solution). The amount of deprotecting base may be any value within the ranges described herein, including end points (e.g., any value within a range of greater than zero to about 5 vol %) and all subranges within the range are also disclosed.

At least a portion (e.g., a majority) of the deprotecting base evaporates (volatizes) during the removing step (e.g., at least a portion, majority, etc. of the deprotecting base evaporates into an upper interior portion of the reaction vessel during the removing step). The process for deprotecting a protected amino acid in accordance with the present disclosure also includes directing (e.g., continuously and/or intermittently directing) inert gas through the reaction vessel to assist in removing (e.g., to assist in flushing, venting, discharging, displacing, replacing, purging, etc., e.g., to remove, flush, vent, discharge, displace, replace, purge, etc.) evaporated (volatized) deprotecting base from the interior of the reaction vessel (e.g., from the headspace of the reaction vessel) during the step of removing the protecting group.

In some embodiments, the directing step can include introducing inert gas into an upper interior portion of the reaction vessel through a first opening located in an upper portion of the reaction vessel; and venting (flushing) inert gas and evaporated deprotecting base from the upper interior portion of the reaction vessel (e.g., from the headspace of the reaction vessel) through a second opening also located in the upper portion of the reaction vessel.

In some embodiments, the directing step can include introducing the inert gas into a lower interior portion of the reaction vessel through an opening located in a lower portion of the reaction vessel; and venting (flushing) the inert gas and evaporated deprotecting base from an upper interior portion of the reaction vessel (e.g., from the headspace of the reaction vessel) through an opening located in an upper portion of the reaction vessel.

In some embodiments, the directing step can include introducing inert gas into an upper interior portion of the reaction vessel through a first opening located in an upper portion of the reaction vessel and also introducing inert gas into a lower interior portion of the reaction vessel through a second opening located in a lower portion of the reaction vessel; and venting inert gas and evaporated deprotecting base from the upper interior portion of the reaction vessel (e.g., from the headspace of the reaction vessel) through a third opening located in the upper portion of the reaction vessel. In this embodiment, inert gas introduced into the lower interior portion of the reaction vessel may serve to agitate (bubble, stir, etc.) materials (e.g., the deprotection reaction mixture, e.g., the deprotection reaction solution) in the lower interior portion of the reaction vessel and/or to participate in headspace venting (flushing, purging, etc.) of evaporated deprotecting base as described herein.

In some embodiments, the process can further include heating the protected amino acid and/or the protected peptide and/or the deprotecting base during the step of removing the protecting group from the protected amino acid and/or the protected peptide. The heating step may be conducted, for example, at a temperature from about 40° C. to about 120° C., as another example about 60° C. to about 120° C., and as another example about 90° C. to about 120° C. The heating step may be conducted using microwave radiation.

In exemplary embodiments of the deprotection processes disclosed herein, the deprotection processes can use (e.g., protected amino acid and/or protected peptide can be attached to) a solid resin support having a resin substitution of less than or about 0.35 mmol/g (e.g., <0.35 mmol/g), for example less than or about 0.30 mmol/g (e.g., <0.30 mmol/g). In some embodiments, the deprotection processes can use a solid resin support with resin substitution from 0.10 mmol/g to 0.35 mmol/g, for example 0.15 mmol/g to 0.35 mmol/g, for example 0.10 mmol/g to 0.34 mmol/g, for example 0.15 mmol/g to 0.34 mmol/g, for example 0.20 mmol/g to 0.35 mmol/g, for example 0.20 mmol/g to 0.34 mmol/g, for example 0.20 mmol/g to 0.33 mmol/g. The amount of resin substitution may be any value within the ranges described herein, including end points and all subranges within the range are also disclosed. In exemplary embodiments, the resin may be PEG-PS (polyethylene glycol-polystyrene) resin (e.g., PEG-PS based resin (Pro-Tide), PS (polystyrene) resin, etc.

The present disclosure also relates to a process for solid phase peptide synthesis (SPPS) and/or liquid phase peptide synthesis (LPPS). The SPPS and/or LPPS process may include deprotecting a first protected amino acid and/or protected peptide to provide a deprotected amino acid and/or deprotected peptide, as described herein; and coupling a second amino acid to the deprotected amino acid to form a peptide from the first and second amino acids and/or coupling an amino acid to the deprotected peptide to form a second peptide from the deprotected peptide and the amino acid.

In some embodiments, the process does not include a washing step after a deprotecting step and before a (next, successive) coupling step of a SPPS cycle.

In some embodiments, the process may include a washing step after a deprotection step and before a (next, successive) coupling step of a SPPS cycle using a washing composition (e.g., solvent). In some embodiments, the process may include a washing step after a deprotection step and before a (next, successive) coupling step of a SPPS cycle using a washing composition (e.g., solvent) in an amount that is about the same as the total volume of the deprotection reaction mixture (e.g., the deprotection reaction solution). In some embodiments, the process may include a washing step after a deprotection step and before a (next, successive) coupling step of a SPPS cycle using a washing composition (e.g., solvent) in an amount that is less than the total volume of the deprotection reaction mixture (e.g., the deprotection reaction solution). For example, the washing step may include washing using a washing composition (e.g., a solvent) in an amount that is less than or about ½ of the total volume of the deprotection reaction mixture (e.g., the deprotection reaction solution), and as another example washing using a washing composition (e.g., a solvent) in an amount that is less than or about ⅓ of the total volume of the deprotection reaction mixture (e.g., the deprotection reaction solution). In some embodiments, the washing step may include washing the interior of the reaction vessel using a total volume of solvent that is 2 times or less of a bed volume of resin present in the reaction vessel (e.g., a solid resin support present in the reaction vessel), for example, 1 times or less of a bed volume of resin present in the reaction vessel.

In some embodiments, the process may include one or more extraction steps after a deprotection step and before a (next, successive) coupling step of a LPPS cycle using an extraction solvent (e.g., an aqueous solvent that is immiscible with the deprotection reaction product, e.g., the growing peptide chain). In some embodiments, the process may include one or more extraction steps after a deprotection step and before a (next, successive) coupling step of a LPPS cycle using a total volume of an extraction solvent (e.g., a total combined volume of extraction solvent for all extraction steps of a single deprotection-coupling cycle) that is two times (2×) or less than the total amount (total volume) of the deprotection reaction mixture in the reaction vessel of that deprotecting-coupling cycle.

### BRIEF DESCRIPTION OF THE DRAWINGS

The present invention is described hereinafter with reference to the accompanying drawings in which embodiments of the present invention are shown and in which like reference numbers may indicate the same or similar elements. The drawings are provided as examples, may be schematic, and are not intended to be drawn to scale. The present inventive aspects may be embodied in many different forms and should not be construed as limited to the examples depicted in the drawings, For purposes of clarity, not every component is labeled in every figure, nor is every component of each embodiment of the invention shown where illustration is not necessary to allow those of ordinary skill in the art to understand the invention.

FIG. 1A is a cross-sectional view of an exemplary reaction vessel and schematically depicts a process of deprotecting a protected amino acid (e.g., in a SPPS process) in accordance with embodiments of the present disclosure.

FIG. 1B is a cross-sectional view of another exemplary reaction vessel and schematically depicts a process of deprotecting a protected amino acid (e.g., in a SPPS process) in accordance with other embodiments of the present disclosure.

FIG. 2A is a schematic flow diagram depicting selected portions of an exemplary peptide synthesis system (e.g., in a SPPS and/or LPPS system system) in accordance with embodiments of the present disclosure.

FIG. 2B is a schematic flow diagram depicting selected portions of another exemplary peptide synthesis system (e.g., a SPPS and/or LPPS system) in accordance with other embodiments of the present disclosure.

FIG. 3 is a flow chart schematically depicting the steps of a cycle of conventional solid phase peptide synthesis (SPPS) processes.

FIGS. 4A and 4B are flow charts schematically depicting the steps of a cycle of SPPS processes in accordance with the present disclosure.

FIG. 4C is a flow chart schematically depicting the steps of a cycle of LPPS processes in accordance with the present disclosure.

FIG. 5A is a cross-sectional view of an exemplary reaction vessel and schematically depicts a process of deprotecting a protected amino acid (e.g., in a LPPS process) in accordance with embodiments of the present disclosure.

FIG. 5B is a cross-sectional view of another exemplary reaction vessel and schematically depicts a process of deprotecting a protected amino acid (e.g., in a LPPS process) in accordance with other embodiments of the present disclosure.

FIG. 6A is a schematic flow diagram depicting selected portions of another exemplary peptide synthesis system (e.g., a SPPS and/or LPPS system) in accordance with embodiments of the present disclosure.

FIG. 6B is a schematic flow diagram depicting selected portions of another exemplary peptide synthesis system (e.g., a SPPS and/or LPPS system) in accordance with other embodiments of the present disclosure.

### DETAILED DESCRIPTION

Examples of embodiments are disclosed in the following. The above and other aspects, features, and advantages of the present invention will become apparent from the detailed description of the following embodiments. The present invention may, however, be embodied in many different forms and should not be construed as limited to the embodiments set forth herein. For example, features disclosed as part of one embodiment or example (e.g., features of one or more of processes of deprotecting a protected amino acid and/or a protected peptide during peptide synthesis including deprotecting steps and coupling steps, solid phase peptide synthesis (SPPS) processes, and/or liquid and/or solution phase peptide synthesis (LPPS) processes, etc.) can be used in the context of another embodiment or example to yield a further embodiment or example. The embodiments are provided for complete disclosure and to provide thorough understanding of the present invention by those skilled in the art. Sometimes well-known aspects are not described in detail to avoid unnecessarily obscuring the present invention. This detailed description is thus not to be taken in a limiting sense, and it is intended that other embodiments are within the spirit and scope of the present invention. The scope of the present invention should be defined only by the appended claims.

Embodiments of the present disclosure relate to processes and systems for deprotecting a protected amino acid (e.g., deprotecting a protected amino acid, deprotecting a protected amino acid-derived unit of a peptide, also referred to herein as deprotecting a protected peptide, wherein the protected peptide refers to a peptide including a unit derived from a protected amino acid) as a step peptide synthesis (e.g., in solid phase peptide synthesis and/or liquid phase peptide synthesis). In exemplary embodiments, the processes and systems are batch-based processes and systems.

The skilled artisan will understand the meaning of the term amino acid. As used herein, the term amino acid in its broadest sense refers to organic compounds that contain both amine and carboxylic acid functional groups, and in some instances also a side chain. The skilled artisan will also understand that amino acids include natural amino acids (proteinogenic amino acids) and/or non-proteinogenic amino acids, and will also understand the single letter designations used to identify the same.

The processes of the present disclosure may be useful in the production of peptides and/or proteins. The terms peptide and/or protein will also be understood by the skilled artisan. For example, as used herein, the term peptide and/or protein can refer to amides derived from two or more amino acids (the same or different) by bonding the carbonyl carbon of one amino acid to the nitrogen atom of another amino acid. As understood by the skilled artisan, peptides and proteins may be distinguished by chain length (e.g., peptides have a shorter chain length of amino acids linked by chemical bonds (fewer amino acids), as compared to proteins). For ease of discussion, however, the term peptide will used consistently throughout, and the present disclosure is not limited to the production of peptides (e.g., the processes described herein may be applicable to the production of peptides and/or proteins).

For ease of reference, the present disclosure refers to processes for deprotecting a protected amino acid (e.g., removing a protecting group of a protected amino acid). The skilled artisan will understand that the protected amino acid may be part of a peptide and that discussions herein to processes for deprotecting a protected amino acid also include processes for deprotecting a protected peptide (e.g., removing a protecting group of a protected amino acid-derived unit of a peptide).

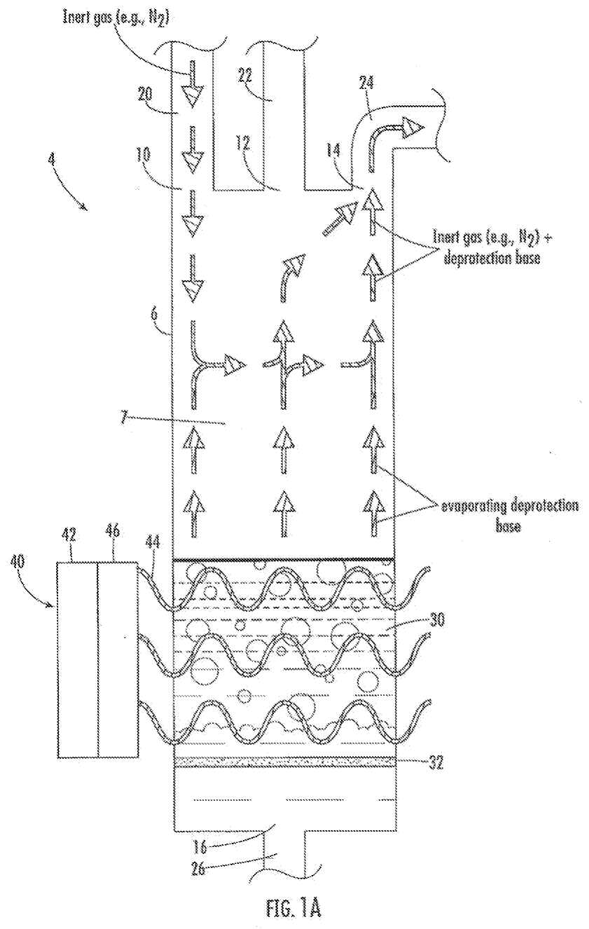

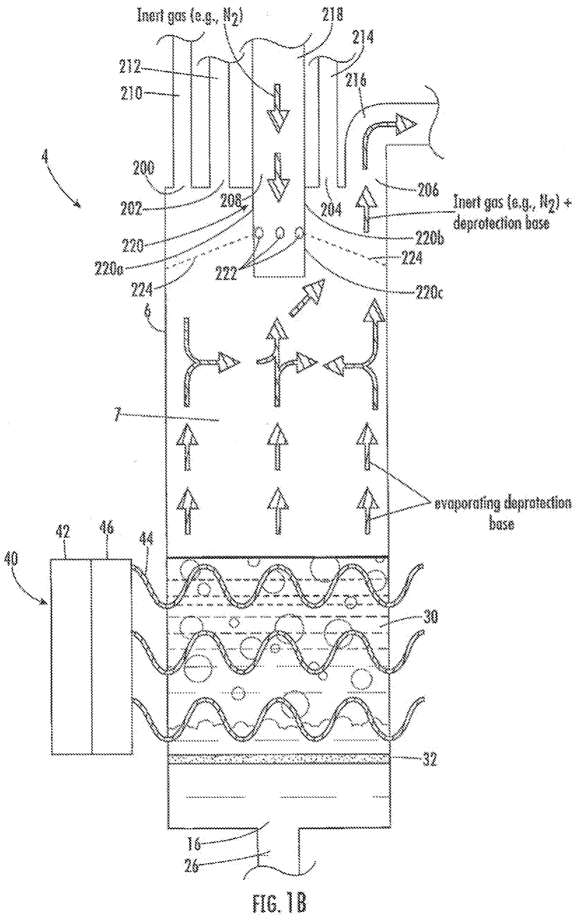

FIG. 1A is a schematic cross-sectional view of a reaction vessel suitable for use in the amino acid deprotecting and peptide synthesis processes and systems (e.g., SPPS processes and systems) in accordance with embodiments of the present disclosure. FIG. 1A also schematically depicts a process (e.g., SPPS) according to an embodiment of the present disclosure for deprotecting a protected amino acid.

FIG. 1B is a schematic cross-sectional view of another reaction vessel suitable for use in the amino acid deprotecting and peptide synthesis processes and systems (e.g., SPPS processes and systems) in accordance with embodiments of the present disclosure. FIG. 1B also schematically depicts a process (e.g., SPPS) according to an embodiment of the present disclosure for deprotecting a protected amino acid.

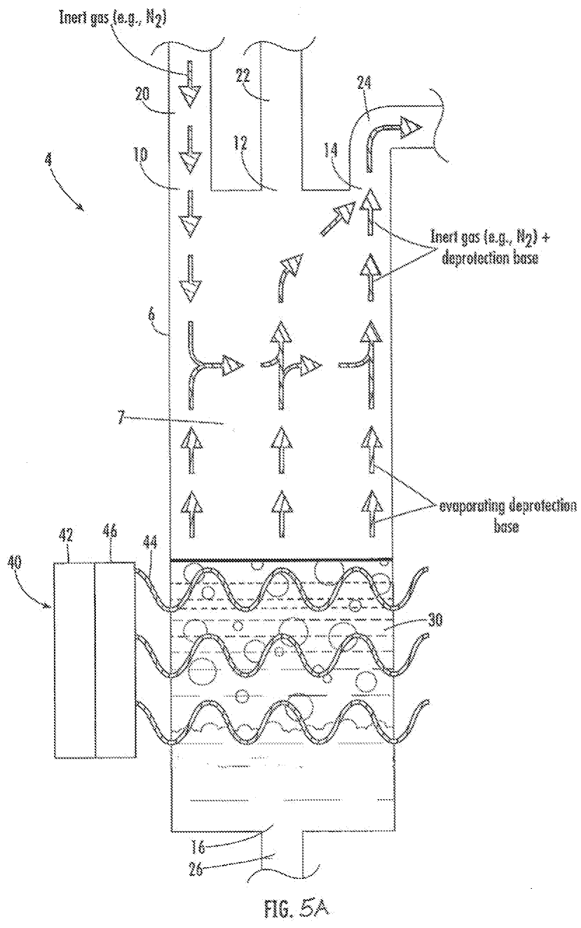

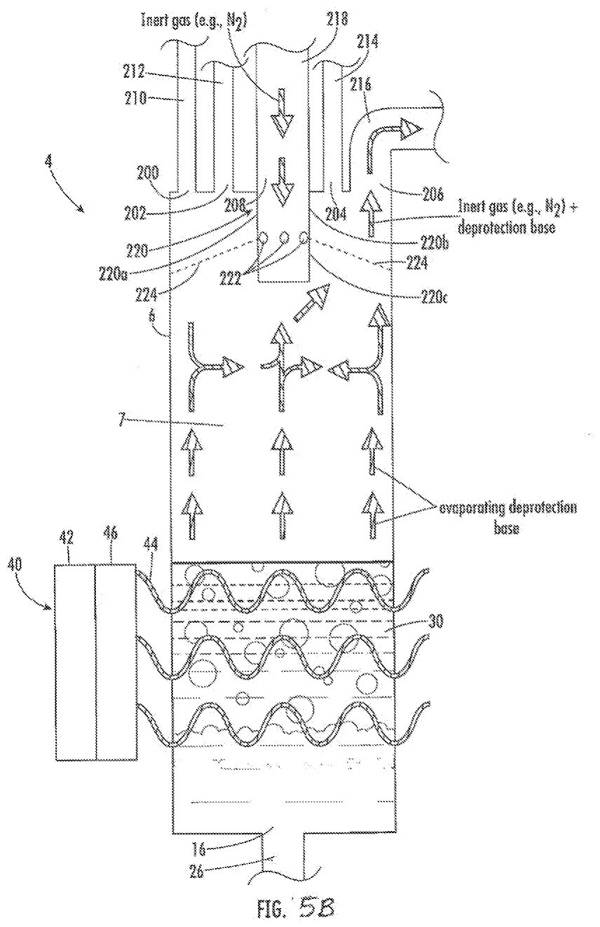

FIGS. 5A and 5B are schematic cross-sectional views of reaction vessels suitable for use in the amino acid deprotecting and peptide synthesis processes and systems (e.g., LPPS processes and systems) in accordance with embodiments of the present disclosure. FIGS. 5A and 5B also schematically depict processes (e.g., LPPS) according to embodiments of the present disclosure for deprotecting a protected amino acid. The skilled artisan will understand that the reaction vessels depicted in FIGS. 5A and 5B can have many, if not most, of the same elements as described herein with regard to the reaction vessels of FIGS. 1A and 1B, except that the reaction vessels for LPPS do not typically include a filter 32 as described herein. As depicted herein, the reaction vessels of FIGS. 5A and 5B useful for LPPS do not include a filter 32 and a mixture 30 also described herein may rest on a lower surface (e.g., bottom wall) of the reaction vessel. A lower portion of the reaction vessels of FIGS. 5A and 5B also may be configured as known in the art to allow extraction (removal) of a waste layer (e.g., an aqueous layer including residual deprotecting base) following deprotection while maintaining a product layer (e.g., an organic layer including a growing peptide chain). Such reaction vessels including suitable extraction mechanisms for use in LPPS processes are known in the art and are not described in detail herein. The skilled artisan will also understand that the reaction vessels of FIGS. 5A and 5B are representative of suitable reaction vessels for LPPS processes, the present disclosure is not limited thereto, and that other commercially available reaction vessels suitable for LPPS processes may be used.

Except where indicated otherwise, elements illustrated in FIG. 1A will carry the same reference numerals as in the other Figures, including FIGS. 1B, 5A, and 5B.

As shown in FIGS. 1A and 1B (and FIGS. 5A and 5B), a reaction vessel 4 includes at least one side wall 6 extending around an interior 7 (e.g., cavity) of reaction vessel 4 (the interior 7 of the reaction vessel also referred to herein as an outer interior space). The specific size and shape of reaction vessel 4 are not limited. Reaction vessels suitable for use in solid phase peptide synthesis and/or liquid phase synthesis are well known in the art and are commercially available.

The size (interior volume) of the reaction vessel is not limited. Exemplary reaction vessel sizes can range from less than 1 liter up to 40 liters, or more, for example, 10 ml, 30 ml, 125 ml, 1 liter, 3 liters, 5 liters, 8 liters, 10 liters, 15 liters, etc. up to 40 liters, or more, without limitation.

Reaction vessel 4 further includes one or more openings. As a non-limiting example, FIG. 1A (and FIG. 5A) depicts openings 10, 12, and 14 located in an upper portion (e.g., in a top wall) of reaction vessel 4, and an opening 16 located in a lower portion (e.g., a bottom wall) of reaction vessel 4. As another non-limiting example, FIG. 1B (and FIG. 5B) depicts openings 200, 202, 204, 206, and 208 located in an upper portion (e.g., in a top wall) of reaction vessel 4, and an opening 16 located in a lower portion (e.g., a bottom wall) of reaction vessel 4.

The openings (e.g., inlets, outlets, ports, etc.) allow the introduction and/or removal of fluids and/or solids, such as reactants, solvents, gases, products (peptides), byproducts, excess (residual) reactants, and the like as discussed in more detail herein.

The skilled artisan will understand that the reaction vessel 4 is not limited to the number and/or locations of openings depicted in FIGS. 1A and 1B (and FIGS. 5A and 5B) and that other reaction vessel designs and configurations having fewer or more openings and/or different locations thereof can be used (e.g., the reaction vessel may include fewer or more openings located in a top wall and/or a bottom wall and/or a side wall, etc.).

Fluids and/or solids can be introduced (e.g., moved, transported, directed, flushed, purged, evacuated, vented, etc.) into and out of reaction vessel 4 via one or more flow paths (e.g., lines, passageways, tubes, manifolds, etc.), such as flow paths 20, 22, and 24 in fluid communication with openings 10, 12, and 14, respectively, and flow path 26 in fluid communication with opening 16, as depicted in FIG. 1A (and FIG. 5A).

Other non-limiting examples of flow paths are depicted in FIG. 1B (and FIG. 5B) as flow paths 210, 212, 214, 216, and 218 in fluid communication with openings 200, 202, 204, 206, and 208, respectively, and flow path 26 in fluid communication with opening 16.

In some embodiments, reaction vessel 4 can include at least one spray head (e.g., spray nozzle) or equivalent structure located in the interior of the reaction vessel for adding (e.g., directing, supplying, spraying, etc.) fluid (e.g., solvent, reactant, and/or inert gas) into the reaction vessel. The spray head can be a part (e.g., component, element, etc.) that is separate from the reaction vessel (e.g., can be installed in and removed from the reaction vessel) or can be an integrated part of the reaction vessel.

As a non-limiting example, FIG. 1B (and FIG. 5B) schematically depicts an embodiment including a spray head 220 located in the interior space 7 (also referred to herein as the outer interior space) of reaction vessel 4. Spray head 220 includes at least one side wall 220a positioned in the interior space 7 (also referred to as the outer interior space) of reaction vessel 4 and extending around an inner interior space. Spray head 220 includes a first portion (end) 220b proximate opening 208 in fluid communication with opening 208 (and flow path 218) and a second portion (end) 220c distal opening 208. A plurality of holes 222 (e.g., ports, etc.) extend at least partially around the inner interior space and can be, for example, defined in side wall 220a. The inner interior space and the outer interior space are in fluid communication with one another by way of holes of the plurality of holes.

Spray head 220 is configured so that fluid (such as inert gas, solvent, etc. as discussed herein) directed into the inner interior space of spray head 220 via flow path 218 and opening 208 passes (e.g., is sprayed, directed, supplied, etc.) through holes (e.g., through one, more than one, a majority, or all) of the plurality of holes 222 into the outer interior space 7 of reaction vessel 4. In some embodiments, spray head 220 can be configured so that fluid exiting at least one or more holes of the plurality of holes is directed (sprayed) towards side wall 6 of the reaction vessel. An exemplary spray pattern is schematically depicted in FIG. 1B (and FIG. 5B) by dashed lines 224 (e.g., in an approximate manner), in which fluid exiting holes of the plurality of holes 222 is directed at a generally downward angle towards side wall 6.

The present disclosure is not limited with respect to a specific spray head structure, location in the reaction vessel, spray pattern and/or direction (angle) of fluid exiting holes of the spray head, such as illustrated in FIG. 1B (and FIG. 5B), and other spray head structures, locations, spray patterns, and/or spray angles, etc., can be used. Spray heads suitable for use in SPPS processes and systems are known in the art and can be used in the present disclosure.

The present disclosure is not limited to a specific number and/or locations of openings and flow paths and the reaction vessel can accordingly have one, two, three, four, or more openings and associated flow paths as appropriate. Further, any series of flow paths and associated valves that serve to direct, allow, and/or block (e.g., close, restrict, etc.) the flow of fluids and/or solids can be used.

Reaction vessel 4 includes a mixture of components, designated generally at 30 in FIGS. 1A and 1B (and FIGS. 5A and 5B), located in a lower portion of reaction vessel 4. In FIGS. 1A and 1i, the mixture of components 30 can rest on a filter 32 located in the lower portion of reaction vessel 4. Filter 32 can also prevent solid materials present in the mixture (such as solid resin support linked to a growing peptide chain as discussed in more detail herein) from entering flow path 26. As noted herein, and as depicted herein, the reaction vessels of FIGS. 5A and 5B useful for LPPS may not include a filter 32 and the mixture 30 may rest on a lower surface (e.g., bottom wall) of the reaction vessel.

For example, mixture 30 includes a protected amino acid, i.e., an amino acid including at least one protecting group attached to a functional group, such as a terminal amine group, to protect from unwanted reactions of the functional group. The protected amino acid may be part of a growing peptide chain linked to a solid resin support and/or other suitable support (e.g., a soluble tag), as understood and known in the art and as discussed in more detail herein.

Suitable protecting groups for use in the processes of the present disclosure are well-known in the art. An example of a protecting group suitable for protection of amine or N-terminus includes without limitation a fluorenylmethyloxycarbonyl (Fmoc) protecting group. See, for example, Chan and White, Fmoc solid phase peptide synthesis, a practical approach, Oxford University Press (2000).

The amino acid can also include side-chain protecting groups. Examples of side-chain protecting groups can include without limitation trityl, t-butyl, and/or 2,2,4,6,7-pentamethyldihydrobenzofuran-5-sulfonyl (Pbf) protecting groups, and the like. When the desired peptide chain length has been obtained, the side-chain protecting groups can be removed.

The protected amino acid can be directly or indirectly attached to a solid support or other suitable support (e.g., a soluble tag for LPPS) as known in the art. For example, the carboxy terminus of the protected amino acid can be attached to the solid support or other suitable support via a suitable linker. As another example, the carboxy terminus of the protected amino acid can be indirectly attached to the solid support or other suitable support, for example, the carboxy terminus of the protected amino acid can be coupled to the amine or N-terminus of another amino acid (e.g., to the amine or N-terminus of a single amino acid or to the amine or N-terminus of an amino acid that is part of a growing peptide chain) that is in turn linked to the solid support or other suitable support via a suitable linker.

Solid supports known in the art may be used in the processes of the present disclosure. In exemplary embodiments, the solid support is a solid resin support. Examples of solid resin support materials may include without limitation polystyrene (e.g., in resin form such as microporous polystyrene resin, mesoporous polystyrene resin, macroporous polystyrene resin), glass, polysaccharides (e.g., cellulose, agarose), polyacrylamide resins, polyethylene glycol, and/or copolymer resins (e.g., comprising polyethylene glycol, polystyrene, etc.).

In exemplary embodiments of the deprotection processes disclosed herein, the protected amino acid can be attached to a solid resin support having a resin substitution of less than or about 0.35 mmol/g (e.g., <0.35 mmol/g), for example less than or about 0.30 mmol/g (e.g., <0.30 mmol/g). In some embodiments, the deprotection processes can use resin with resin substitution from 0.10 mmol/g to 0.35 mmol/g, for example 0.15 mmol/g to 0.35 mmol/g, for example 0.10 mmol/g to 0.34 mmol/g, for example 0.15 mmol/g to 0.34 mmol/g, for example 0.20 mmol/g to 0.35 mmol/g, for example 0.20 mmol/g to 0.34 mmol/g, for example 0.20 mmol/g to 0.33 mmol/g, for example 0.21 mmol/g to 0.35 mmol/g, for example 0.21 mmol/g to 0.34 mmol/g, for example 0.21 mmol/g to 0.33 mmol/g, for example 0.22 mmol/g to 0.35 mmol/g, for example 0.22 mmol/g to 0.34 mmol/g, for example 0.22 mmol/g to 0.33 mmol/g. The amount of resin substitution may be any value within the ranges described herein, including end points and all subranges within the range are also disclosed. In exemplary embodiments, the resin may be PEG-PS (polyethylene glycol-polystyrene) resin (e.g., PEG-PS based resin (Pro-Tide), PS (polystyrene) resin, etc. In exemplary embodiments, the solid support is a solid PEG-PS (polyethylene glycol-polystyrene) resin support (e.g., PEG-PS based resin (Pro-Tide) at moderate substitution (e.g., 0.2-0.3 mmol/g, 0.20-0.25 mmol/g, 0.21-0.3 mmol/g, 0.21-0.25 mmol/g, 0.22-0.3 mmol/g, 0.22-0.25 mmol/g, etc.).

The solid support may have any suitable form. For example, the solid support can be in the form of beads, particles, fibers, and/or in any other suitable form.

The present disclosure is not limited to solid support materials described herein, and other types of support and/or carrier and/or protecting group materials known in the art may also be used in the deprotecting processes during peptide synthesis including deprotecting and coupling steps, SPPS processes, LPPS processes, etc. described herein.

In exemplary embodiments including a deprotecting step of a LPPS process, a carboxy or C-terminus of an amino acid and/or peptide may be linked to a suitable support and/or carrier and/or protecting group, and the N-terminus may also include a protecting group (e.g., a Fmoc protecting groups).

As a non-limiting example, in some embodiments, soluble tags (e.g., soluble polymer tags (also referred to as soluble resin tags) and/or soluble compound tags, etc.) known in the art can be used as a carrier and/or support and/or protecting group (e.g., a carboxyl or C-terminus protecting group of an amino acid and/or peptide) in deprotecting processes during peptide synthesis including deprotecting and coupling steps, such as LPPS processes, etc. described herein. In some embodiments of the present disclosure (e.g., LPPS processes) protected amino acid(s), singly or as part of a (growing) peptide chain, may be attached to one or more soluble tags (e.g., soluble polymer tags, soluble compound tags, etc. as known in the art). Soluble tags (e.g., soluble polymer tags, soluble compound tags, etc.) can include polymers and/or compounds that can precipitate under certain conditions and dissolve in other conditions as known in the art. Thus, as known in the art, soluble tags (e.g., soluble polymer tags and/or soluble compound tags) useful in embodiments of the present disclosure (e.g., LPPS processes) can have a property of reversibly changing between solid-phase state and liquid-phase state depending on conditions (e.g., solvent, reagent, temperature, etc.). Generally, in solution phase peptide synthesis processes of the present disclosure, the soluble tag and amino acid and/or peptide chain linked thereto is in a solution or liquid phase state during deprotecting and/or coupling steps.

Exemplary soluble polymer tags and/or soluble compound tags can include one or more functional groups (e.g., alcohol functional groups, amine functional groups, etc.) that can react with a carboxy or amino terminus of an amino acid and/or peptide to link the amino acid and/or peptide to the soluble polymer tag and/or soluble compound tag. In exemplary embodiments of the present disclosure, the soluble tag is linked to the carboxyl (C-terminus) of the amino acid and/or peptide (which can have also a protected N-terminus, such as a Fmoc protected N-terminus).

Soluble tags (e.g., soluble polymer tags, soluble compound tags, etc.) may be used in the deprotecting processes during peptide synthesis including deprotecting and coupling steps, LPPS processes, etc. described herein (e.g., used in liquid-phase states during deprotecting and/or coupling steps as described herein). Soluble tags can adopt solution phase kinetics for reaction steps (e.g., deprotecting and/or coupling steps as described herein) which can allow for lower reagent equivalents to be used.

Suitable soluble tags (e.g., soluble polymer tags, soluble compound tags, etc.) are known in the art and/or are commercially available and the skilled artisan will understand how to use the same in deprotecting processes during peptide synthesis including deprotecting and coupling steps, such as LPPS processes, etc. as described herein, including suitable conditions to promote solid-phase and/or liquid-phase states thereof in deprotecting and/or coupling steps.

For example, soluble compound tags may include aromatic compounds (e.g., benzyl compounds) with functional group(s) (e.g., alcohol and/or amine functional groups), hydrophobic group(s) (e.g., groups including a long chain alkyl component), etc. Exemplary soluble compound tags may include, for example, hydrophobic benzyl alcohol or amine type compounds as shown below:

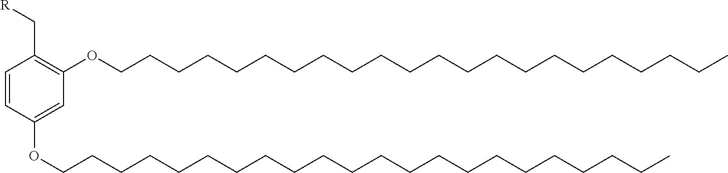

wherein R represents a group including one or more atoms (e.g., oxygen nitrogen, sulfur, etc.) that can react with a carboxy or amide terminus of an amino acid and/or peptide to link the polymer tag and amino acid and/or peptide. A typical example of a soluble compound tag may include the compound above wherein R═OH or R═NH.

The skilled artisan will understand how link (couple) an amino acid and/or peptide to a support (e.g., a solid resin support and/or a soluble tag). Accordingly, a detailed discussion of methods known in the art for linking (coupling) an amino acid and/or peptide to a solid support and/or a soluble tag is not provided.

Turning again to FIGS. 1A and 1B (and FIGS. 5A and 5B), mixture 30 also includes a deprotection reaction mixture (e.g., a deprotection reaction solution). The deprotection reaction mixture (e.g., the deprotection reaction solution) may include a mixture (e.g., may include the total volume of liquid in the reaction vessel) including a deprotection solution including a deprotecting base (e.g., a deprotection solution including a deprotecting base added to the reaction vessel) and a coupling solution from a preceding coupling step (e.g., an undrained post-coupling solution that remains in the reaction vessel from a preceding (previous) coupling step). In some embodiments, the deprotection reaction mixture (e.g., the deprotection reaction solution) may include additional liquid(s) (e.g., additional solvent added to the reaction vessel).

In this regard, as discussed in more detail herein, the process for deprotecting a protected amino acid in accordance with the present disclosure may include adding a deprotection solution that includes the deprotecting base to the reaction vessel. The amount of the deprotection solution including the deprotecting base added to the reaction vessel corresponds to the deprotection solution including a deprotecting base of the deprotection reaction mixture (e.g., the deprotection reaction solution).

Also in this regard, the process for deprotecting a protected amino acid in accordance with the present disclosure may include a preceding coupling step (e.g., a coupling step prior to the step of removing the protecting group). In exemplary embodiments, the reaction vessel may include various components after completion of the coupling step. The components in the reaction vessel post-coupling are referred to herein generally as a post-coupling mixture.

For example, as understood in the art, the reaction vessel may include a coupling solution after completion of the preceding coupling step. In exemplary embodiments, the process of the present disclosure does not require draining step(s) (and/or washing step(s)) after the preceding coupling step and before the next (e.g., successive) deprotecting step (e.g., the deprotecting step of the next SPPS and/or LSSP cycle) to remove (e.g., drain) the coupling solution from the reaction vessel. When there is no draining step between a preceding coupling step and a successive deprotecting step, the deprotection reaction mixture (e.g., the deprotection reaction solution) in the reaction vessel includes the coupling solution remaining in the reaction vessel from the preceding coupling step.

Thus, the post-coupling mixture may include a post-coupling solution (also referred to herein as a coupling solution). For example, the reaction vessel may include a coupling solution (e.g., undrained post-coupling solution) that remains in the reaction vessel from the previous coupling step. The components of the coupling solution (e.g., undrained post-coupling solution) will be understood by the skilled artisan. For example, the coupling solution (e.g., the undrained post-coupling solution) may include solvent and residual (excess) coupling reagents and/or coupling reagent byproducts from the preceding coupling reaction. Examples of residual (excess) coupling reagents and/or byproducts thereof that may be present in the coupling solution include without limitation residual (excess) activated amino acid, residual (excess) amino acid activator (e.g., DIC, etc., as described herein), residual (excess) amino acid activator additive (e.g., Oxyma, etc., as described herein), other residual (excess) coupling additives, non-activated protected amino acids, and/or byproducts thereof, without limitation. The skilled artisan will understand the meaning of the term coupling and/or post-coupling solution as used herein and the definition thereof is not necessarily limited to the components described herein.

As another example, the skilled artisan will also understand that the post-coupling mixture in the reaction vessel may include solids, such as post-coupling reaction products (e.g., a growing peptide chain(s) including two or more amino acids coupled to a solid support resin and/or a soluble polymer support) that are not part of the coupling solution (e.g., the undrained post-coupling solution that remains in the reaction vessel from the previous coupling step). The post-coupling mixture, however, in some embodiments may include post-coupling reaction products in a solution or liquid phase state (e.g., a peptide chain including two or more amino acids coupled to a soluble tag that is in a solution or liquid phase state). The deprotection reaction mixture (e.g., deprotection reaction solution) in some embodiments may accordingly also include post-coupling reaction products that are in a liquid or solution phase state. Again, the skilled artisan will understand the meaning of the term post-coupling reaction products as used herein and the definition thereof is not necessarily limited to the components described herein.

In exemplary embodiments, the process of the present disclosure does not include a draining step between the preceding coupling step and the next deprotecting step (e.g., the deprotecting step of the next SPPS and/or LSSP cycle) to remove (drain) the coupling solution from the reaction vessel. When there is no draining step between a preceding coupling step and a successive deprotecting step, the coupling solution remaining in the vessel from the preceding coupling step corresponds to the coupling solution of the deprotection reaction mixture (e.g., the deprotection reaction solution).

In exemplary embodiments, the process of the present disclosure may include a draining step between the preceding coupling step and the next deprotecting step (e.g., the deprotecting step of the next SPPS and/or LSSP cycle). When there is a draining step between a preceding coupling step and a successive deprotecting step, some of (e.g., less than half of, half of, substantially all, all of, etc.) the coupling solution from the preceding coupling step may be drained from the reaction vessel and, in some embodiments, the deprotection reaction mixture (e.g., the deprotection reaction solution) may include minimal, if any, coupling solution (e.g., undrained post-coupling solution) from the preceding coupling step, depending on the amount of post-coupling solution drained from the reaction vessel. When post-coupling solution is drained from the reaction vessel between the preceding coupling step and the next deprotecting step, the volume percent deprotecting base in the reaction vessel may still be the same as described in more detail herein (e.g., an amount greater than zero to about 5 vol % based on the total volume (100 vol %) of the deprotection reaction mixture (e.g., the deprotection reaction solution)). In some embodiments, much of the total volume of the deprotection reaction mixture (e.g., the deprotection reaction solution) (e.g., half of, substantially of, all of, etc.) may correspond& substantially to the volume of deprotection solution added to the reaction vessel, alone or in combination with a volume of additional liquid (e.g., solvent) added to the reaction vessel. For example, in some embodiments in which substantially all (e.g., all) of a post-coupling solution is drained from the reaction vessel prior to the next deprotecting step, a deprotection solution including greater than zero to about 5 vol % deprotecting agent, and optionally additional solvent, may be added to the drained reaction vessel to provide the deprotecting base in the reaction vessel in an amount of greater than zero to about 5 vol %, based on the total volume (100 vol %) of the deprotection reaction mixture (e.g., the deprotection reaction solution) (which in this case, the volume of deprotection reaction mixture (e.g., the deprotection reaction solution) may substantially correspond to the total volume of the added deprotection solution and additional solvent when present) in the reaction vessel. As another example, in some embodiments in which substantially all (e.g., all) of a post-coupling solution is drained from the reaction vessel prior to the next deprotecting step, a deprotection solution including deprotecting agent in a vol % greater than about 5 vol % and additional solvent may be added to the drained reaction vessel to provide the deprotecting base in the reaction vessel in an amount of greater than zero to about 5 vol %, based on the total volume (100 vol %) of the deprotection reaction mixture (e.g., the deprotection reaction solution) (which in this case, the volume of deprotection reaction mixture (e.g., the deprotection reaction solution) may substantially correspond to the total volume of the added deprotection solution and the added solvent) in the reaction vessel.

The process for deprotecting the protected amino acid and/or protected peptide in accordance with the present disclosure includes removing the protecting group of the protected amino acid and/or the protected peptide in a reaction vessel such as reaction vessel 4 with the deprotecting base, wherein the deprotecting base is present in the reaction vessel in an amount greater than zero to about 5 vol %, based on the total volume (100 vol %) of the deprotection reaction mixture (e.g., the total volume of the deprotection reaction solution, total volume of liquid) in the reaction vessel. The deprotecting base reacts with the protected amino acid and/or protected peptide to remove the protecting group and to make the previously protected functional group (e.g., a terminal amine group) available for reaction (e.g., with one, two, or more, successive amino acid to form a peptide chain).

The deprotection reaction mixture (e.g., the deprotection reaction solution) may include a mixture (e.g., a total volume of liquid in the reaction vessel) including a deprotection solution including the deprotecting base (e.g., a deprotection solution including the deprotecting base added to the reaction vessel) and a coupling solution from a preceding coupling step (e.g., an undrained post-coupling solution remaining in the reaction vessel from a preceding coupling step). In some embodiments, the deprotection reaction mixture (e.g., the deprotection reaction solution) may also include additional liquid (e.g., solvent) added to the reaction vessel and/or post-coupling reaction products that are in a liquid or solution phase state. In some embodiments, the vol % deprotecting base of the deprotection reaction mixture (e.g., the deprotection reaction solution) may be based on the total volume of liquid in the reaction vessel (e.g., the deprotection reaction mixture or solution may include the total volume of liquid in the reaction vessel). For example, in some embodiments, when the coupling solution from a preceding coupling step is not drained, the vol % deprotecting base may be based on the total volume of liquid in the reaction vessel including the volume of a coupling solution from a preceding coupling step (e.g., the volume of an undrained post-coupling solution remaining in the reaction vessel from a preceding coupling step, not including solids, for example not including a growing peptide chain attached to a solid support) and/or post-coupling reaction products that are in a liquid or solution phase state. and the volume of the added deprotection solution including the deprotecting base (and volume of additional solvent when added to the reaction vessel). In some other embodiments, when the coupling solution from a preceding coupling step is partially or substantially completely drained (e.g., less than half of, half of, substantially all of, or all of coupling solution is drained), the vol % deprotecting base may be based on the total volume of liquid in the reaction vessel including the volume of the added deprotection solution including the deprotecting base (and volume of additional solvent when added to the reaction vessel) and any remaining (e.g., greater than half of, half of, substantially no or minimal (residual) or no) volume coupling solution and/or post-coupling reaction products that are in a liquid or solution phase state (e.g., from the preceding coupling step).

In some embodiments, the deprotecting base may be present in the reaction vessel in an amount of about 1 vol % to about 5 vol %, for example about 2 vol % to about 5 vol %, for example from about 2 vol % to about 4.5 vol %, for example from about 3 vol % to about 4.5 vol %, and as another example from about 3.5 vol % to about 4.5 vol %, based on the total volume (100 vol %) of the deprotection reaction mixture (e.g., the total volume of the deprotection reaction solution, total volume of liquid) in the reaction vessel. In some embodiments, the deprotecting base may be present in the reaction vessel in an amount greater than zero to about 4.5 vol %, for example about 2 vol % to about 4.5 vol %, based on the total volume of the deprotection reaction mixture (e.g., the total volume of the deprotection reaction solution, total volume of liquid) in the reaction vessel. In some embodiments, the deprotecting base may be present in the reaction vessel in an amount greater than zero to about 4 vol %, for example about 2 vol % to about 4 vol %, based on the total volume of the deprotection reaction mixture (e.g., the total volume of the deprotection reaction solution, total volume of liquid) in the reaction vessel. In some embodiments, the deprotecting base may be present in the reaction vessel in an amount greater than zero to about 3.5 vol %, for example about 2 vol % to about 3.5 vol %, based on the total volume of the deprotection reaction mixture (e.g., the total volume of the deprotection reaction solution, total volume of liquid) in the reaction vessel. The amount of deprotecting base may be any value within the ranges described herein, including end points (e.g., any value within a range of greater than zero to about 5 vol %) and all subranges within the range are also disclosed.

Deprotecting bases used in SPPS and/or LSSP processes are typically liquid at room temperature. Thus, the deprotecting base is typically added to the reaction vessel as a part of a deprotection solution that includes the deprotecting base and a suitable solvent. Alternatively, in some embodiments, the deprotecting base may be added neat to the reaction vessel.

In exemplary embodiments, the process of the present disclosure includes adding the deprotection solution including the deprotecting base (alone or in combination with a volume of additional solvent) to the reaction vessel under conditions sufficient to provide a desired amount (e.g., volume) of the deprotecting base in the reaction vessel to be available for removing the protecting group of the protected amino acid (e.g., to provide greater than zero to about 5 vol % deprotecting base in the reaction vessel, based on the total volume (100 vol %) of the deprotection reaction mixture (e.g., total volume of deprotection reaction solution, total volume of liquid) in the reaction vessel). In exemplary embodiments, this may include adding the deprotection solution (and optionally additional solvent) to the reaction vessel in an amount (volume) sufficient and/or adding deprotection solution having a concentration of the deprotecting base sufficient to provide the desired amount (volume) of the deprotecting base in the reaction vessel available for removing the protecting group of the protected amino acid (e.g., to provide greater than zero to about 5 vol % deprotecting base in the reaction vessel, based on the total volume (100 vol %) of the deprotection reaction mixture (e.g., deprotection reaction solution, liquid) in the reaction vessel). The deprotection solution added to the reaction vessel may include the deprotecting base in a higher concentration than the resultant concentration of the deprotecting base in the reaction vessel after the deprotection solution is added to the reaction vessel.

As a non-limiting example, following completion of a small-scale coupling reaction, a reaction vessel may include about 3.5 mL post-coupling solution (including solvent, residual (excess) activated amino acid, residual (excess) amino acid activator such as DIC, and/or residual (excess) amino acid activator additive such as Oxyma). A deprotecting step may be initiated by adding 0.75 mL of a 17% v/v pyrrolidine/DMF deprotecting solution (0.1275 mL of pyrrolidine) directly to the undrained post-coupling solution to give a total volume of 4.25 mL deprotection reaction mixture (e.g., deprotection reaction solution) in the reaction vessel, including about 3 vol % pyrrolidine based on the total volume of the deprotection reaction mixture (e.g., the deprotection reaction solution).

The deprotection solution to be added to the reaction vessel can have a higher concentration of the deprotecting base, as compared to the concentration of the deprotecting base in the deprotection reaction mixture (e.g., the deprotection reaction solution) after deprotection solution is added to the reaction vessel. Thus, a relatively small amount (volume) of the deprotection solution may be added to the reaction vessel to provide the desired deprotecting base concentration of the deprotection reaction mixture (e.g., the deprotection reaction solution).

In some embodiments, as used herein, a deprotection solution added to the reaction vessel having a “high” or “higher” concentration of deprotecting base may include a deprotection solution including solvent and about 10 vol % or higher deprotecting base, about 15 vol % or higher deprotecting base, about 20 vol % or higher deprotecting base, about 25 vol % or higher deprotecting base, about 30 vol % or higher deprotecting base, about 40 vol % or higher deprotecting base, and less than 50 vol % deprotecting base, based on the total volume of the deprotection solution. In some embodiments, the deprotection solution added to the reaction vessel may include the deprotecting base in an amount from about 10 vol % to about 40 vol %, for example from about 10 vol % to about 30 vol %, and as another example from about 10 vol % to about 25 vol %, based on the total volume of the deprotection solution. In some embodiments, the deprotection solution added to the reaction vessel may include the deprotecting base in an amount of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 vol %, based on the total vol (100 vol %) of the deprotection solution. Further, according to some embodiments, the deprotecting base can be present in an amount of from about any of the foregoing amounts to about any other of the foregoing amounts. In some other embodiments, a deprotection solution added to the reaction vessel having a “high” or “higher” concentration of deprotecting base may include a deprotection solution including solvent and deprotecting base in an amount of about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 vol %, based on the total vol (100 vol %) of the deprotection solution. Further, according to some embodiments, the deprotecting base can be present in an amount of from about any of the foregoing amounts to about any other of the foregoing amounts.

The present disclosure, however, is not limited to the use of a deprotection solution having relatively high concentrations of deprotecting base, and the process may also include adding a deprotection solution to the reaction vessel having a concentration of deprotecting base that is less than about 10 vol % (e.g., the deprotection solution added to the reaction vessel may include solvent and the deprotecting base, wherein the deprotecting base is present in an amount greater than zero vol % to about 10 vol %, for example greater than zero vol % to about 5 vol %, based on the total volume of the deprotection solution), so long as the amount of the deprotection solution added to the reaction vessel is selected to provide the desired amount (greater than zero to about 5 vol %) of deprotecting base, relative to the total volume of the deprotection reaction mixture (e.g., the deprotection reaction solution) as defined herein. In some embodiments, the deprotection solution added to the reaction vessel may include the deprotecting base in an amount of about 1, 2, 3, 4, 5, 6, 7, 8, 9, or 10 vol %, based on the total vol (100 vol %) of the deprotection solution. Further, according to some embodiments, the deprotecting base can be present in an amount of from about any of the foregoing amounts to about any other of the foregoing amounts.

The deprotecting base can be an organic base. In some embodiments, the deprotecting base can have a boiling point of less than or about 107° C. In some embodiments, the deprotecting base can have a boiling point of less than or about 107° C. and/or a difference between a deprotection reaction temperature such as discussed herein and a boiling point of the deprotecting base may be less than or about 50° C., for example less than or about 25° C., for example less than or about 15° C., for example the difference between the deprotection reaction temperature and the boiling point of the deprotecting base may range from about 1° C. to about 50° C., for example the difference between the deprotection reaction temperature and the boiling point of the deprotecting base may range from about 15° C. to about 50° C., for example the difference between the deprotection reaction temperature and the boiling point of the deprotecting base may range from about 1° C. to about 35° C., and as another example the difference between the deprotection reaction temperature and the boiling point of the deprotecting base may range from about 1° C. to about 25° C. In some embodiments, the deprotecting base can have a boiling point of less than or about 107° C. and/or the difference between the deprotection reaction temperature and boiling point of the deprotecting base may be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50° C. The difference between the deprotection reaction temperature and boiling point of the deprotecting base may be any value within the ranges described herein, including end points (e.g., any value within a range of about 1° C. to about 50° C.) and all subranges within the range are also disclosed. In some embodiments, the deprotecting base can have a boiling point of less than or about 107° C. and/or the deprotection step may be performed at a temperature of no less than about 35° C. below the boiling point of the deprotection base used. Examples of an organic base suitable for use as a deprotecting base may include without limitation piperidine and/or pyrrolidine. Other organic bases that provide the deprotection function without otherwise interfering with the other steps in the process, the growing peptide chain, or the system, can be appropriate as well.

Examples of solvents that can be part of the deprotection solution (and/or may be added separately to a reaction vessel) may include without limitation dimethylformamide (DMF), dimethylacetamide (DMA), N-methylpyrrolidinone (NMP), green solvents and/or non-reprotoxin solvents, and the like, and combinations and/or mixtures thereof. Examples of green and/or non-reprotoxin solvents may include without limitation N-formylmorpholine (NFM), N-butylpyrrolidinone (NBP), alkoxybenzene-based solvents (e.g., anisole, dimethoxybenzene-based solvents such as 1,3-dimethoxoybenzene, etc.), and the like, and combinations and/or mixtures thereof.

Referring again to FIGS. 1A and 1B (and FIGS. 5A and 5B), mixture 30 is typically initially present in a lower interior portion of a reaction vessel. At least a portion of the deprotecting base present in mixture 30 evaporates during the removing step (e.g., at least a portion of the deprotecting base evaporates into an upper interior portion of the reaction vessel during the removing step). For example, the deprotecting base can have a lower boiling point relative to the deprotection reaction temperature/temperature of the reaction vessel during deprotection and/or a lower boiling point relative to the boiling point of solvent(s) that may be present in the reaction vessel (e.g., solvents present in the deprotection reaction mixture (e.g., the deprotection reaction solution)), such as dimethylformamide (DMF) and N-methylpyrrolidinone (NMP). Accordingly, the deprotecting base can volatize or evaporate during the deprotection process.

In some embodiments, the deprotecting step (e.g., the deprotection reaction step) can be conducted without heat (e.g., can be conducted at room temperature), so long as the deprotecting conditions (type of base, time, etc.) are selected to promote evaporation of the deprotecting base (e.g., into the headspace of the reaction vessel).

More typically, in some embodiments, the deprotecting step/process may be conducted with heat (e.g., the deprotecting step/process may include heating the protected amino acid and/or the protected peptide and/or the deprotecting base (e.g., heating the deprotection solution including the deprotecting base) and/or other liquids (e.g., additional added solvent, residual coupling solution, etc.) and/or the reaction vessel, etc.). The protected amino acid and/or the protected peptide and/or the deprotecting base and/or other liquids and/or the reaction vessel, etc. may be heated before and/or during the step of removing the protecting group from the protected amino acid and/or the protected peptide. For example, the process may include heating the protected amino acid and/or the protected peptide and/or the deprotecting base and/or other liquids such as added solvent before and/or during delivery into the reaction vessel 4 and/or heating the protected amino acid and/or the protected peptide and/or the deprotecting base and/or other liquids in the reaction vessel 4 before and/or during the deprotection reaction (e.g., by heating the reaction vessel before and/or during the step of removing the protecting group from the protected amino acid and/or the protected peptide). Heating during solid phase peptide synthesis can be useful, for example, to accelerate the rate of deprotection and thereby reduce the amount of time required for peptide synthesis.

Heating temperatures of the heating step may vary. In some embodiments, the heating step (e.g., heating during the deprotecting step/process) can be conducted at a temperature from about 40° C. to about 120° C., for example from about 50° C. to about 120° C., as another example from about 60° C. to about 120° C., as another example from about 70° C. to about 120° C., as another example from about 80° C. to about 120° C., as another example from about 90° C. to about 120° C., as another example from about 80° C. to about 110° C., and as another example from about 90° C. to about 110° C., without limitation. In certain embodiments, the heating step (e.g., heating during the deprotecting step/process) can be conducted at a temperature from about 60° C. to about 120° C., for example from about 90° C. to about 120° C., and as another example from about 90° C. to about 110° C., without limitation. The temperature may be any value within the ranges described herein, including end points (e.g., any value within a range of from about 40° C. to about 120° C.) and all subranges within the range are also disclosed.

FIGS. 1A and 1B (and FIGS. 5A and 5B) schematically depict a heating step wherein a heat source 40 heats reaction vessel 4 and mixture 30. In certain embodiments, heat source 40 includes a microwave source 42 positioned to direct microwave radiation 44 through a waveguide 46 attached to a microwave cavity (not illustrated) containing reaction vessel 4. Microwave power may be regulated as known in the art to provide reaction temperatures and/or reaction times (e.g., without limitation, to provide deprotection temperatures as described herein and to provide deprotection reaction times ranging from about 10 sec to about 15 minutes, as another non-limiting example from about 40 sec to about 8 minutes).

In embodiments utilizing microwave energy to heat the reactants, reaction vessel 4 can be formed of a material that is transparent to microwave radiation, such as but not limited to glass, Teflon, and/or polypropylene.

Microwave sources are well known in the art and can include, for example, magnetrons, klystrons, and/or solid-state diodes. Microwave sources, waveguides and microwave cavities suitable for solid phase peptide synthesis processes and systems are well known in the art and also are commercially available (e.g., systems commercially available from CEM Corporation such as discussed herein). Accordingly, the skilled artisan will understand how to use the same in solid phase peptide synthesis processes and systems without undue experimentation.

The present disclosure, however, is not limited to the use of microwave sources as the heat source, and other types of heat sources known in the art for solid phase peptide synthesis can used.

Despite the benefits of heating, elevated temperatures during the deprotecting step can present various challenges for peptide synthesis.

For example, organic amines used in deprotection reactions can have relatively low boiling points, as compared to the boiling point of a solvent used in a deprotection reaction and/or the temperature of the deprotecting step. Piperidine has a boiling point of about 106° C. and pyrrolidine has a boiling point of about 87° C. In contrast, the solvent dimethylformamide (DMF) has a boiling point of about 153° C. and the solvent N-methylpyrrolidinone (NMP) has a boiling point of about 200° C. Also in contrast, as noted herein, deprotecting reactions can be conducted at elevated temperatures, for example up to about 120° C., for example about 90° C. to about 120° C., and as another example about 90° C. to about 110° C., without limitation.

A reaction vessel can exhibit a temperature continuum during processing, wherein an upper portion thereof can be at a lower temperature than lower portions. Because the deprotecting base can have a boiling point lower than the boiling point of other components present in the reaction vessel such as solvents and/or lower than reaction temperatures, the deprotecting base can volatize (evaporate) into the upper portion of the reaction vessel (e.g., the headspace) and then condense on upper portions of the reaction wall(s) and/or on a top wall of the reaction vessel. The rate/amount of volatilization (evaporation) can also increase, for example, when the reactants are bubbled during deprotection (e.g., using an inert gas such as nitrogen) to help mix the reactants.

Volatilization of a deprotecting base can be especially problematic using pyrrolidine. Pyrrolidine would be desirable as a deprotecting base because pyrrolidine can provide faster deprotection than piperidine. As a 5-membered ring (versus a 6-membered piperidine ring), the carbon atoms of pyrrolidine are bent back more from the nitrogen atom, which facilitates an easier attack for deprotection. Because pyrrolidine has a lower boiling point than piperidine, however, significant evaporation followed by condensation can occur during deprotection processes, thereby limiting its use, including without example in the synthesis of long peptides.

In the processes of the present disclosure, the heating step can volatize (evaporate) the deprotecting base (e.g., pyrrolidine) in the deprotection reaction mixture (e.g., the deprotection reaction solution) from the lower portion of reaction vessel 4 upwardly into the upper portion (e.g., into the headspace above mixture 30) of reaction vessel 4.

Residual deprotecting base remaining in a reaction vessel (e.g., residual deprotecting base condensed on upper portions of the reaction wall(s) and/or on a top wall of the reaction vessel) during subsequent solid phase peptide synthesis steps (e.g., a subsequent coupling step) can be problematic. Residual deprotecting base can, for example, prematurely remove a protecting group from an amino acid to be coupled to the already deprotected amino acid. This can result in undesirable insertions into the peptide chain. Residual deprotecting base can also reduce activated amino acid by reacting with the amino acid, which can result in deletions in the peptide chain.

Accordingly, conventional SPPS processes required washing steps after deprotection and before coupling (e.g., to help remove residual deprotecting base to minimize or prevent participation thereof in subsequent solid phase peptide synthesis steps such as a subsequent coupling step).

In contrast to conventional SPPS processes, the processes of the present disclosure can help eliminate washing step(s) between deprotecting and coupling steps and/or reduce the amount of solvent required for a washing step(s) between deprotecting and coupling steps of a SPPS process.

Further, as known in the art, conventional LPPS processes required extraction step(s) after deprotection and before coupling. Extraction steps include adding a suitable extraction solvent such as water to the reaction vessel including a peptide product (e.g., a growing peptide chain) and residual deprotecting base. This results in the formation of separate layers, including an organic layer including the peptide product and an aqueous layer including waste products such as residual deprotecting base. The waste layer (e.g., the aqueous laying including residual base) can be drained from the reaction vessel while maintaining the organic product layer including a growing peptide chain in the reaction vessel using techniques known in the art. Extraction processes are used to separate a peptide product and waste products, such as residual deprotecting base, to minimize or prevent participation of the base in subsequent liquid phase peptide synthesis steps such as a subsequent coupling step.

Nonetheless, conventional LPPS processes typically require several extraction steps and/or significant amounts of extraction solvent to remove residual base, and even then the remaining organic layer including the peptide product can be contaminated with waste products (e.g., can include higher than desired amounts of deprotecting base, etc.).

In contrast to conventional LPPS processes, in some embodiments, the LPPS processes of the present disclosure can help reduce the volume (amount) of extraction solvent required after deprotection and before the next successive coupling step and/or help remove a significant portion of residual deprotecting base remaining after completion of the deprotection from the reaction vessel.

To help eliminate washing step(s) between deprotecting and coupling steps and/or reduce the amount of solvent required for a washing step(s) between deprotecting and coupling steps of a SPPS process, and/or to help reduce the amount of solvent used in an extraction step(s) after deprotection and before the next successive coupling step in a LPPS process, the process of the present disclosure uses small amounts of deprotecting base as described herein and/or directs (e.g., continuously and/or intermittently directs) an inert gas through the interior of the reaction vessel (e.g., directs inert gas through the upper interior portion, or headspace, of the reaction vessel) during the deprotection step to assist in removing (e.g., to assist in flushing, venting, discharging, displacing, replacing, purging, etc., e.g., to remove, flush, vent, discharge, displace, replace, purge, etc.) evaporated (volatized) deprotecting base from the interior of the reaction vessel (e.g., from the reaction vessel headspace).

The processes of the present disclosure may also generally facilitate the production of peptides having acceptable purity levels for downstream applications.

In some embodiments, the step of directing an inert gas through the interior of the reaction vessel may include directing (introducing, supplying, flowing, etc.) inert gas into an upper interior portion of the reaction vessel via one or more openings (entry ports) located in an upper portion of the reaction vessel so that inert gas flows through the upper interior portion of the reaction vessel and out of the upper interior portion of the reaction vessel through one or more other openings (exit ports) located in the upper portion of the reaction vessel. In this manner, inert gas may flow through the upper interior portion of the reaction vessel including evaporated deprotecting base (e.g., through the vessel headspace) and assist in removing (e.g., assist in flushing, venting, discharging, displacing, replacing, purging, etc., e.g., to remove, flush, vent, discharge, displace, replace, purge, etc.) evaporated deprotecting base from the upper interior portion (e.g., from the headspace above mixture 30) of the reaction vessel through one or more other openings (exit ports) located in the upper portion of the reaction vessel.

In some embodiments, the step of directing an inert gas through the interior of the reaction vessel may include directing (introducing, supplying, flowing, etc.) inert gas into a lower interior portion of the reaction vessel via one or more openings (entry ports) located in a lower portion of the reaction vessel so that inert gas flows upwardly from the lower interior portion of the reaction vessel into/through the upper interior portion of the reaction vessel and out of the upper interior portion of the reaction vessel through one or more other openings (exit ports) located in the upper portion of the reaction vessel. In this manner, inert gas may flow upwardly from the lower interior portion of the reaction vessel into/through the upper interior portion of the reaction vessel including evaporated deprotecting base (e.g., through the vessel headspace) and assist in removing (e.g., assist in flushing, venting, discharging, displacing, replacing, purging, etc., e.g., to remove, flush, vent, discharge, displace, replace, purge, etc.) evaporated deprotecting base from the upper interior portion (e.g., from the headspace above mixture 30) of the reaction vessel through one or more other openings (exit ports) located in the upper portion of the reaction vessel.

In some embodiments, the step of directing an inert gas through the interior of the reaction vessel may include directing (directing, supplying, flowing, etc.) inert gas into both an upper interior portion and a lower interior portion of the reaction vessel via one or more openings (entry ports) located in an upper portion and a lower portion of the reaction vessel, respectively so that inert gas flows through the upper interior portion of the reaction vessel (including optionally upwardly from the lower interior portion of the reaction vessel into/through the upper interior portion of the reaction vessel) and out of the upper interior portion of the reaction vessel through one or more other openings (exit ports) located in the upper portion of the reaction vessel. In this manner, inert gas may flow through the upper interior portion of the reaction vessel including evaporated deprotecting base (e.g., through the vessel headspace) and may also optionally flow upwardly from the lower interior portion of the reaction vessel into/through the upper interior portion of the reaction vessel including evaporated deprotecting base (e.g., through the vessel headspace) and assist in removing (e.g., assist in flushing, venting, discharging, displacing, replacing, purging, etc., e.g., to remove, flush, vent, discharge, displace, replace, purge, etc.) evaporated (volatized) deprotecting base from the upper interior portion (e.g., from the headspace above mixture 30) of the reaction vessel through one or more other openings (exit ports) located in the upper portion of the reaction vessel.

In some embodiments, the inert gas can be continuously directed through the reaction vessel as a continuous flow. In some embodiments, the inert gas can be directed through the reaction vessel as an intermittent (e.g., pulsed) flow.

In some embodiments, inert gas directed into and/or flowing through the reaction vessel may have a pressure of about 1 psi to about 25 psi. In some embodiments, inert gas directed into and/or flowing through the reaction vessel (e.g., flowing through the headspace of the reaction vessel including volatized deprotecting base; and/or bubbling through mixture 30 and/or flowing upwardly through mixture 30 of the reaction vessel) may have a pressure of about 1, 2, 3, 4, 5, 6, 7, 8, 9, 10, 11, 12, 13, 14, 15, 16, 17, 18, 19, 20, 21, 22, 23, 24, or 25 psi. In some embodiments, inert gas directed into and/or flowing through the reaction vessel may have a pressure in a range from about any of the foregoing pressure values to about any other of the foregoing pressure values. The pressure of the inert gas, including inert gas directed into and/or flowing through an upper interior portion of the reaction vessel and/or directed into and/or flowing through a lower interior portion of the reaction vessel (including directed into and/or flowing through mixture 30, e.g., bubbling through and/or flowing generally upwardly through mixture 30 into the upper interior portion of the reaction vessel), may be any value within the ranges described herein, including end points (e.g., any value within a range of from about 1 to about 25 psi) and all subranges within the range are also disclosed.

In some embodiments, the pressure of inert gas directed into and/or flowing through the reaction vessel may be higher than about 25 psi. As a non-limiting example, in some large-scale production methods (e.g., large scale microwave peptide synthesizer production methods) including deprotection step(s) according to the present disclosure (e.g., including one or more deprotecting steps as described herein using a deprotecting base in an amount from greater than zero to about 5 vol %, based on the total volume of the deprotection reaction mixture (e.g., the deprotection reaction solution) and/or using an inert gas to flush volatized deprotecting base from the reaction vessel (e.g., from the head space of the interior of the reaction vessel), also as described in more detail herein), the inert gas may have a pressure from about 1 psi to about 100 psi, for example from greater than or about 25 psi to about 100 psi, for example from about 50 psi to about 95 psi, and as another example from about 75 psi to about 95 psi. In some embodiments, a large scale production method including deprotection step(s) according to the present disclosure may employ a reaction vessel having a size (e.g., interior volume) of 3 liters or larger (e.g., 3 liters, 8 liters, 10 liters, 15 liters, etc., up to 40 liters or larger), and/or may have a synthesis scale of about 25 mmol or higher (e.g., about 25 mmol or higher, about 50 mmol or higher, about 100 mmol or higher, about 200 mmol or higher, about 250 mmol or higher, about 500 mmol or higher., etc.), and/or may provide peptide quantities per batch of up to about 500 grams or higher (e.g., about 500 grams or higher, about 1 kg or higher, etc.), and/or have varying deprotection cycle times (e.g., from about 8 minutes to about 15 minutes, about 10 minutes, etc.).

In some embodiments in which inert gas is introduced into the reaction vessel both via a first opening located in an upper portion of the reaction vessel (the upper opening) and a second opening located in a lower portion of the reaction vessel (the lower opening), inert gas flowing into and/or through the upper opening and/or upper interior portion of the reaction vessel may have a higher psi than inert gas flowing into and/or through the lower opening and/or lower interior portion of the reaction vessel.

The inert gas may also be directed through the interior (e.g., through an upper interior portion, or headspace) of the reaction vessel during the deprotection step at a flow rate based on a time rate within which the inert gas substantially replaces (displaces) the headspace gas volume. More specifically, the inert gas flow rate may be an amount (volume) of inert gas that allows for (results in) substantial replacement (displacement) of the volume of gas in the headspace area of the reaction vessel with (by) the inert gas (e.g., that results in substantial replacement of the volume of volatized deprotecting base in the headspace area of the reaction vessel with the inert gas) within a selected time period (time rate). For example, the inert gas flow rate may be an amount (volume) of inert gas that results in (allows for) the substantial replacement (displacement) of the volume of gas in the headspace area (e.g., the volume of volatized deprotecting base in the headspace area) of the reaction vessel about every one (1) to twenty (20) seconds, for example, about every five (5) to ten (10) seconds. The skilled artisan will understand how to determine and calculate suitable inert gas flow rates to replace (displace) a volume of headspace gas (volatized deprotecting base) in a reaction vessel within a time frame (time rate) without undue experimentation.

Although not wishing to be bound by any explanation or theory, it is currently believed that directing a source of inert gas into and/or through the headspace during deprotection can cause a large air exchange rate in the gas above the deprotection reaction mixture (e.g., the deprotection reaction solution) and/or other reactants, products, etc. in a lower interior portion of the reaction vessel (e.g., in the headspace gas including the volatized deprotecting base) such that the inert gas displaces the volatized deprotecting base from the reaction vessel. This can reduce residence time of the volatized deprotecting base in the reaction vessel and the volatized deprotecting base can be more quickly removed with less condensation on the side and/or top walls of the vessel. This in turn can help reduce the amount of residual deprotecting base remaining in the reaction vessel after the deprotecting step is completed. The inert gas can also provide downward force on droplets (e.g., condensed deprotecting base) on a side wall of reaction vessel 4 and can thereby blow the droplets toward mixture 30 in the lower portion of reaction vessel 4.

Because the deprotecting process uses a low amount of deprotecting base (less than or about 5 vol % deprotecting base, based on the total volume of deprotection reaction mixture (e.g., the deprotection reaction solution)), the deprotecting base (e.g., pyrrolidine) may be essentially completely removed from the reaction vessel upon completion of the deprotection step. For example, without being bound by any theory or explanation, in such embodiments, it is currently believed that the deprotecting base may substantially completely evaporate during a heating step and/or volatized deprotecting base may be substantially completely removed from the headspace using inert gas flushing, each as described herein. Also without being bound by any theory or explanation, in such embodiments, it is currently believed that any residual amount of deprotecting base remaining after completion of the deprotection step is small enough to minimize issues associated with the presence of residual deprotecting base in the next coupling step, even without a washing step after the deprotection step and/or with a washing step after the deprotection step using amounts of washing liquid (e.g. solvent) and/or using reduced amounts of extraction solvent after a deprotection step in a LPPS process, also as described in more detail herein.

Thus, in exemplary embodiments, the deprotecting processes described herein can remove a significant portion of the deprotecting base used for the deprotection step from the reaction vessel (e.g., by evaporation). As used herein, a “significant portion of the deprotecting base” can include without limitation at least a majority (e.g., more than half), at least about 60%, at least about 70%, at least about 80%, at least about 90%, at least about 95%, at least about 99%, or more, of the deprotecting base used for the deprotection step from the reaction vessel. For example, in some embodiments, the deprotecting processes described herein can remove at least about 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of the deprotecting base used for the deprotection step from the reaction vessel. Further, according to some embodiments, the amount of deprotecting base removed from the reaction vessel can be an amount of from about any of the foregoing amounts to about any other of the foregoing amounts.

Because the amount of residual deprotecting base can be reduced, in contrast to conventional approaches, the process of the present disclosure may also facilitate the production of peptides having acceptable purity levels for downstream applications.

The inert gas can be nitrogen. The present disclosure is not limited to the use of nitrogen as the inert gas and other inert gases, such as the noble gases, with limited or no interference chemically with solid phase peptide synthesis reactions and with the solid phase peptide synthesis system can be used.

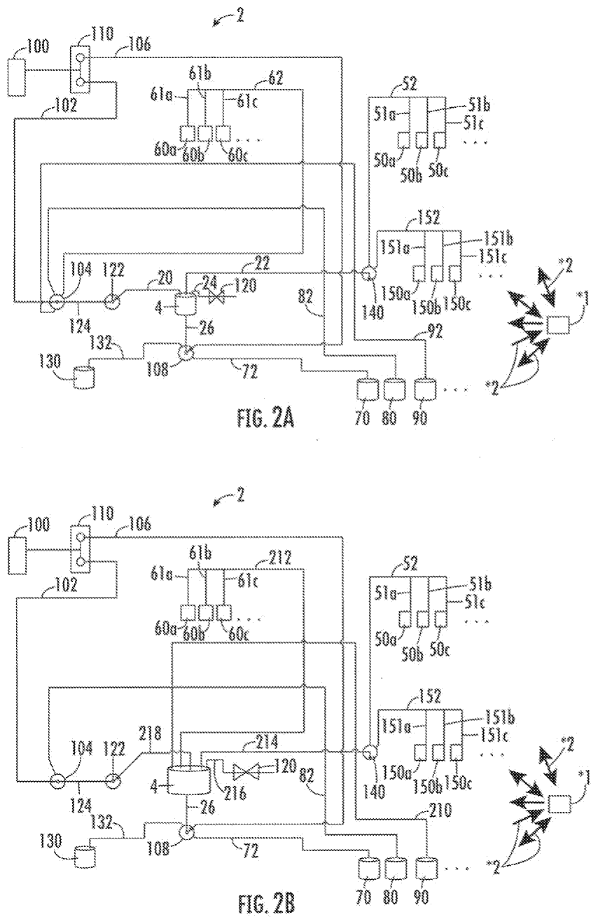

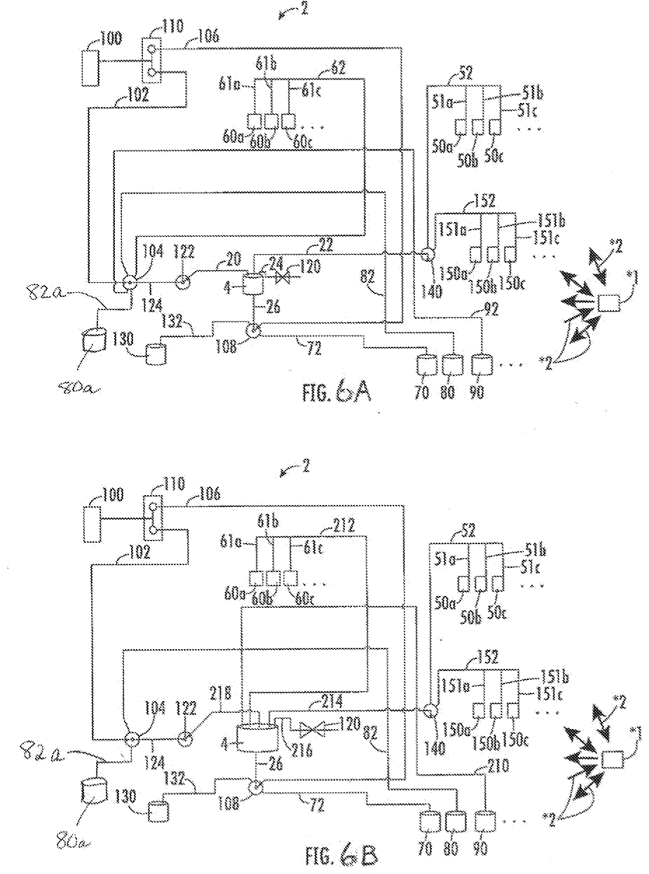

In certain embodiments, as depicted in FIG. 1A (and FIG. 5A), the process can include providing pressurized inert gas from an inert gas source (such as inert gas source designated as 100 in FIGS. 2A and 6A) through flow path 20 and directing the pressurized inert gas (e.g., generally downwardly directing the pressurized inert gas) into the upper portion of the interior of the reaction vessel 4 through opening 10 and into the headspace above mixture 30 (including the deprotection reaction mixture (e.g., the deprotection reaction solution) and protected amino acids) located in a lower interior portion of the reaction vessel. As the pressurized inert gas flows through the headspace from the upper portion of the interior of the reaction vessel 4, the inert gas purges (e.g., removes, flushes, displaces, replaces, vents, discharges, etc.) volatized deprotecting base out of the interior of the reaction vessel 4 through opening 14 into flow path 24. In this manner, the pressurized inert gas in effect displaces the volatized deprotecting base from the headspace of the reaction vessel. This can reduce residence time and minimize condensation of the deprotecting base on the walls of the reaction vessel.

Gas flow (movement) in reaction vessel 4, including upward flow of volatized deprotecting base (e.g., pyrrolidine) from mixture 30 in the lower interior portion of reaction vessel 4 into the headspace above the mixture (e.g., into the upper interior portion of reaction vessel 4), the flow of inert gas downwardly into and through the headspace (upper interior portion) of reaction vessel 4, and purging (e.g., removing, flushing, displacing, venting, discharging, replacing, etc.) of volatized deprotecting base and inert gas from the headspace (upper interior portion) of reaction vessel 4, is schematically depicted by the arrows in FIG. 1A (and FIG. 5A).

In certain embodiments, as depicted in FIG. 1B (and FIG. 5B), the process can include providing (e.g., directing) pressurized inert gas from an inert gas source (such as inert gas source designated as 100 in FIGS. 2B and 6B) through flow path 218, opening 208, the inner interior space of spray head 220, and out openings 222 into outer interior space 7 of reaction vessel 4 (e.g., into the headspace above mixture 30). FIG. 1B (and FIG. 5B) also depicts embodiments wherein the spray head 220 directs (e.g., sprays) inert gas through openings 222 at an angle (e.g., spray pattern) schematically depicted by dashed lines 224 towards side wall 6 of the reaction vessel 4. This can facilitate a washing effect, wherein the inert gas can contact the side wall and “wash” condensed deprotecting base toward reactants in the lower portion of the interior of the reaction vessel 4.

As the pressurized inert gas flows through the headspace of reaction vessel 4, the inert gas purges (e.g., removes, flushes, displaces, replaces, vents, discharges, etc.) volatized deprotecting base out of reaction vessel 4 through opening 206 into flow path 216. Again, the pressurized inert gas in effect displaces the volatized deprotecting base from the headspace of the reaction vessel, which may reduce residence time and minimize condensation of the deprotecting base on the walls of the reaction vessel.

Gas flow (movement) in reaction vessel 4, including upward flow of volatized deprotecting base (e.g., pyrrolidine) from mixture 30 in the lower interior portion of reaction vessel 4 into the headspace above the mixture (e.g., into the upper interior portion of reaction vessel 4), flow of inert gas from spray head 220 through openings 222 (e.g., angled flow towards side wall 6) into and through the headspace (e.g., the upper interior portion) of the reaction vessel, and purging (e.g., flushing, displacement, venting, removing, discharging, etc.) of volatized deprotecting base and inert gas from the headspace (e.g., the upper interior portion) of reaction vessel 4, is schematically depicted by the arrows and dashed lines in FIG. 1B (and FIG. 5B).

In certain embodiments, as discussed herein, the process may include introducing (directing, etc.) an inert gas into a lower interior portion of reaction vessel 4, in addition to or as an alternative to introducing (directing, etc.) an inert gas into an upper interior portion (e.g., into the headspace) of the reaction vessel. For example, referring to FIGS. 1A and 1B (and FIGS. 5A and 5B), the process can include directing a pressurized inert gas from an inert gas source (which can be the same as or different from an inert gas source of an inert gas introduced into the upper interior portion of the reaction vessels when present) through flow path 26 and into the lower interior portion of reaction vessel 4 through opening 16. The pressurized inert gas may flow upwardly from the lower interior portion of the reaction vessel (e.g., generally upwardly through mixture 30) and into/through the upper interior portion (e.g., headspace) of the reaction vessel 4 including volatized (evaporated) deprotecting base. As the inert gas flows upwardly (e.g., through the headspace), the inert gas may purge (e.g., flush, displace, replace, vent, discharge, remove, etc.) volatized deprotecting base from the upper interior portion (e.g., the headspace) of the reaction vessel 4 through opening 14 into flow path 24. Again, in this manner, the pressurized inert gas may displace the volatized deprotecting base from the headspace of the reaction vessel, which may reduce residence time and minimize condensation of the deprotecting base on the walls of the reaction vessel.

The pressurized inert gas introduced into the lower interior portion of the reaction vessel may in addition, or alternatively, agitate (mix, bubble, etc.) mixture 30.

In some embodiments, the process may include introducing (directing) both a first pressurized inert gas into an upper interior portion (e.g., the headspace) of the reaction vessel including evaporated deprotecting base and a second pressurized inert gas into a lower interior portion of a reaction vessel. As non-limiting examples, referring to FIGS. 1A and 1B (and FIGS. 5A and 5B), the process may include directing the first pressurized inert gas into the upper interior portion of reaction vessel 4 through a first opening located in an upper portion of the reaction vessel, such as opening 10 of FIG. 1A (and FIG. 5A) or opening 208 and openings 222 of FIG. 1B (and FIG. 5B), and directing the second pressurized inert gas into the lower interior portion of the reaction vessel 4 through a second opening located in a lower portion of the reaction vessel such as opening 16 of FIGS. 1A and 1B (and FIGS. 5A and 5B). The first pressurized inert gas may flow through the upper interior portion (e.g., through the headspace) of the reaction vessel including evaporated deprotecting base. The second pressurized inert gas may flow through the mixture 30 to agitate (stir, bubble, etc.) the mixture 30 and/or flow generally upwardly from the lower interior portion of the reaction vessel through mixture 30 and into/through the upper interior portion (e.g., the headspace) of the reaction vessel including evaporated deprotecting base. The first pressurized inert gas and optionally the second pressurized inert gas may purge (e.g., flush, displace, replace, vent, discharge, remove, etc.) volatized deprotecting base from the upper interior portion (e.g., from the headspace) of the reaction vessel, for example, through a third opening located in an upper portion of the reaction vessel, such as opening 14 or 206 of FIGS. 1A and 1B (and FIGS. 5A and 5B), respectively.

The first inert gas (also referred to herein as the overhead inert gas) directed into and/or flowing through the upper interior portion (e.g., the headspace above mixture 30) of reaction vessel 4 (e.g., the first inert gas directed through a first opening located in an upper portion of the reaction vessel, such as opening 10 of FIG. 1A (and FIG. 5A) or opening 208 and openings 222 of FIG. 1B (and FIG. 5B)) may have a higher pressure than the second inert gas directed into and/or flowing through the lower interior portion (and optionally upwardly into the headspace) of reaction vessel 4. As a non-limiting example, the first (overhead) inert gas directed into and/or flowing through the upper interior portion (e.g., flowing through the headspace) of reaction vessel 4 can have a pressure from about 1 psi to about 25 psi. In some embodiments, the first (overhead) inert gas directed into and/or flowing through the upper interior portion of reaction vessel 4 can have a pressure of about 1, 2, 3, 4, 5, 6, 7, 8, 9, 10, 11, 12, 13, 14, 15, 16, 17, 18, 19, 20, 21, 22, 23, 24, or 25 psi. Further, according to some embodiments, the first (overhead) inert gas directed into and/or flowing through the upper interior portion of the reaction vessel 4 can have a pressure in a range from about any of the foregoing pressure values to about any other of the foregoing pressure values.

As a non-limiting example, the second inert gas directed into and/or flowing through the lower interior portion of the reaction vessel 4 (and optionally upwardly into the headspace) can have a pressure that is less than the pressure of the first (overhead) inert gas directed into and/or flowing through the upper interior portion of the reaction vessel 4. For example, the second inert gas may have a pressure from about 1 psi to about 25 psi, so long as the pressure of the second inert gas is less than the pressure of the first inert gas. In embodiments wherein an inert gas is introduced only into the lower interior portion of the reaction vessel (there is no inert gas introduced into an upper interior portion of the reaction vessel), the inert gas may also have a pressure from about 1 psi to about 25 psi.

In a non-limiting example, the first (overhead) inert gas directed into and/or flowing through the upper interior portion (e.g., head space) of reaction vessel 4 can have a pressure of about 15 psi and the second inert gas introduced into and/or flowing through the lower portion of the reaction vessel 4 (and optionally upwardly into the headspace) can have a pressure that is less than the pressure of the first (overhead) inert gas directed into and/or flowing through the upper portion of the reaction vessel 4, such as a pressure of about 5 psi.

The process accordingly may allow the use of deprotecting bases with relatively lower boiling points at higher temperatures to accelerate reaction times, while minimizing (reducing) adverse effects associated with using a low boiling point, readily volatized reactant.

After deprotecting is completed, the inert gas flow may be stopped and a coupling step may be conducted using processes as known in the art.

The present disclosure also relates to solid phase peptide synthesis and/or liquid phase peptide synthesis processes including one or more deprotecting steps as described in more detail herein (e.g., including one or more deprotecting steps as described herein using a deprotecting base in an amount of about 5 vol % or less, based on the total volume of the deprotection reaction mixture (e.g., the deprotection reaction solution), and/or using an inert gas to flush volatized deprotecting base from the reaction vessel (e.g., from the head space of the interior of the reaction vessel), also as described in more detail herein). The solid phase peptide synthesis and/or liquid phase peptide synthesis process of the present disclosure further includes one or more coupling steps (e.g., may include one or more deprotecting-coupling cycles). Solid phase peptide synthesis and/or liquid phase peptide synthesis coupling steps and systems for conducting the same are generally known in the art and accordingly are not described in detail herein.

Conventional SPPS processes require multiple washing steps between deprotection and coupling steps (e.g., after deprotection and before coupling) to remove residual deprotecting base. In some embodiments of the present disclosure (e.g., SPPS processes including a deprotection step as described herein), a washing liquid (e.g., a solvent such as but not limited to dimethylformamide (DMF), methanol and/or isopropanol) can be added to the reaction vessel for a washing step after deprotection. The washing step may include a single washing step or the washing step may be carried out repetitively (e.g., with two, three, four, five, etc., repetitions).

Washing steps, however, can require the use of large amounts of solvent, necessitate solvent recovery and disposal, etc. This can increase material costs and peptide synthesis times, decrease efficiencies, etc. In addition, multiple washing steps may be less effective in preventing unwanted reactions and reducing impurities, particularly as peptide length increases, which can make it difficult to synthesize peptides with purities acceptable for downstream applications.

In contrast to conventional processes, in some embodiments, the present disclosure is directed to a SPPS process including a deprotecting step followed by a coupling step, wherein the SPPS process does not include a washing step after the deprotecting step and before the associated coupling step. Stated differently, the process of the present disclosure may eliminate one or more washing steps (e.g., may eliminate all washing steps) between a deprotection step and its associated coupling step (i.e., the coupling step immediately following the deprotection step). This can provide benefits such as improved process efficiencies, energy savings, reduced amounts of solvent required in the SPPS process, reduced material costs, reduced solvent disposal issues, etc.

For example, the SPPS process of the present disclosure may include a series of deprotection-coupling cycles, wherein one or more (e.g., all) washing step(s) are eliminated (e.g., there is no washing step) between the deprotection step and the coupling step of at least one of the deprotection-coupling cycles of the SPPS process. In other examples, the SPPS process of the present disclosure may include a series of deprotection-coupling cycles, wherein one or more (e.g., all) washing step(s) are eliminated (e.g., there is no washing step) between the deprotection step and the coupling step of more than one of the deprotection-coupling cycles, for example for half of the deprotection-coupling cycles, for example for a majority of the deprotection-coupling cycles, and as another example for all of deprotection-coupling cycles, of the SPPS process.

In yet other embodiments, the present disclosure is directed to a SPPS process including a deprotecting step followed by a coupling step, wherein the SPPS process includes one or more washing steps (e.g., one, two, three, four, five, etc. washing steps) using a washing composition (e.g., a solvent) after the deprotecting step and before the associated coupling step. In contrast to conventional washing steps, however, the washing step(s) of this embodiment may use reduced amounts of solvent as compared to conventional SPPS processes.

In some embodiments, the washing step(s) after deprotection and before coupling may include washing the interior of the reaction vessel one or more times (e.g., one, two, three, four, five, etc. times) using a washing composition (e.g., a solvent) in an amount (volume) that is about the same as the total amount (total volume) of the deprotection reaction mixture (e.g., the deprotection reaction solution). In addition, the SPPS process may include a series of deprotection-coupling cycles, wherein one or more of (e.g., half of, a majority of, or all of) the deprotection-coupling cycles include one or more washing steps (e.g., one, two, three, four, five, etc. washing steps) between the deprotection step and the coupling step, and wherein the washing step(s) uses a washing composition (e.g., solvent) in an amount (volume) that is about the same as the total amount (total volume) volume of the deprotection reaction mixture (e.g., the deprotection reaction solution).

In some embodiments, the washing step(s) after deprotection and before coupling may include washing the interior of the reaction vessel using a washing composition (e.g., a solvent) in an amount (volume) that is less than the total amount (total volume) of the deprotection reaction mixture (e.g., the deprotection reaction solution). For example, the washing step can include washing the interior of the reaction vessel one or more times (e.g., one, two, three, four, five, etc. times) using a washing composition (e.g., a solvent) in an amount (volume) that is less than or about ½ of the total amount (total volume) of the deprotection reaction mixture (e.g., the deprotection reaction solution). As another non-limiting example, the process can include washing the interior of the reaction vessel one or more times (e.g., one, two, three, four, five, etc. times) using a washing composition (e.g., a solvent) in an amount (volume) that is less than or about ⅓ of the total amount (total volume) of the deprotection reaction mixture (e.g., the deprotection reaction solution). In some embodiments, the process can include washing the interior of the reaction vessel one or more times (e.g., one, two, three, four, five, etc. times) using a washing composition (e.g., a solvent) in an amount (total volume) that is 2 times or less of a bed volume of a resin present in the reaction vessel (e.g., solid support resin as described herein present in the reaction vessel), for example, 1 times or less of a bed volume of a resin present in the reaction vessel. The skilled artisan will understand that the term “bed volume” refers to the area of the reaction vessel taken up (occupied) by a resin present in the reaction vessel (e.g., solid support resin as described herein present in the reaction vessel) and that a total volume of solvent that is 2 times or less of the bed volume of the resin present in the reaction vessel, for example, 1 times or less of the bed volume of the resin present in the reaction vessel, refers to a volume of liquid (solvent) that fills up this same area (e.g., fills up 2 times or less of the bed volume of the resin present in the reaction vessel, for example 1 times or less of the bed volume of the resin present in the reaction vessel, etc.). In addition, the SPPS process may include a series of deprotection-coupling cycles, wherein one or more of (e.g., half of, a majority of, or all of) the deprotection-coupling cycles include one or more washing steps (e.g., one, two, three, four, five, etc. washing steps) between the deprotection step and the coupling step, and wherein the washing step(s) uses a washing composition (e.g., solvent) in an amount (volume) that is less than the total amount (total volume) of the deprotection reaction mixture (e.g., the deprotection reaction solution) (for example, in an amount that is less than or about ½ of the total volume of the deprotection reaction mixture (e.g., the deprotection reaction solution), as another example in an amount that is less than or about ⅓ of the total volume of the deprotection reaction mixture (e.g., the deprotection reaction solution), and as another example in an amount (total volume) that is 2 times or less of the bed volume of the resin present in the reaction vessel, for example, 1 times or less of the bed volume of the resin present in the reaction vessel.

When used, the washing composition (washing liquid) can include a solvent such as but not limited to dimethylformamide (DMF), methanol and/or isopropanol.

Conventional LPPS processes require one or more extraction steps between deprotection and coupling steps (e.g., after deprotection and before coupling) to remove residual deprotecting base. In some embodiments of the present disclosure (e.g., LPPS processes including a deprotection step as described herein), an extraction solvent (e.g., an aqueous solvent) can be added to the reaction vessel for one or more extraction step(s) after deprotection. The extraction step may include a single extraction step or the extraction step may be carried out repetitively (e.g., with two, three, four, five, etc., repetitions).

Extraction steps, however, can require the use of large amounts of solvent, necessitate solvent recovery and disposal, etc. This can increase material costs and peptide synthesis times, decrease efficiencies, etc. In addition, multiple extraction steps may be less effective in preventing unwanted reactions and reducing impurities, particularly as peptide length increases, which can make it difficult to synthesize peptides with purities acceptable for downstream applications.

Exemplary embodiments of the present disclosure are directed to a LPPS process including a deprotecting step followed by a coupling step, wherein the LPPS process includes one or more extraction steps (e.g., one, two, three, four, five, etc. extraction steps) using an extraction solvent (e.g., water) after the deprotecting step and before the associated (next) coupling step. Extraction steps according to the processes of the present disclosure include adding a suitable extraction solvent such as water to the reaction vessel after the deprotecting step and before the associated (next) coupling step. The reaction vessel includes a peptide product (e.g., a growing peptide chain) and residual deprotecting base. Adding the extraction solvent to the reaction vessel (and optionally shaking the vessel) results in the formation of separate layers, including an organic layer including the peptide product and an aqueous layer including waste products such as residual deprotecting base. The waste layer (e.g., the aqueous laying including residual base) can be formed and drained from the reaction vessel while maintaining the organic product layer including a growing peptide chain in the reaction vessel using techniques known in the art. In contrast to conventional extraction steps, however, the extraction step(s) of this embodiment may use reduced amounts of solvent as compared to conventional LPPS processes.

In some embodiments, the LPPS process includes one or more extraction step(s) (e.g., one, two, three, four, five, etc. extracting steps) after a deprotection step and before the next successive coupling step, wherein the total volume of extraction solvent used in all extraction steps associated with the deprotection-coupling cycle (the total combined volume of extraction solvent for all extraction steps of a single deprotection-coupling cycle) is 2 times (2×) or less than the total amount (total volume) of the deprotection reaction mixture (e.g., the deprotection reaction solution) of the deprotection step of that deprotecting-coupling cycle. In addition, the LPPS process may include a series of deprotection-coupling cycles, wherein one or more of (e.g., half of, a majority of, or all of) the deprotection-coupling cycles include extraction step(s) (e.g., one, two, three, four, five, etc. extraction steps) after a deprotection step and before the next successive coupling step, wherein the total volume of extraction solvent used in all extraction steps associated with a deprotection-coupling cycle (the total combined volume of extraction solvent for all extraction steps of a single deprotection-coupling cycle) is 2 times (2×) or less than the total amount (total volume) of the deprotection reaction mixture (e.g., the deprotection reaction solution) of the deprotection step of that deprotecting-coupling cycle.

When a washing and/or extraction step is used, in some embodiments, the washing liquid (e.g., solvent) and/or extraction solvent (e.g. aqueous solvent) can be introduced into the reaction vessel via a suitable opening into an upper interior portion of the reaction vessel, such as opening 10 of FIG. 1A (and FIG. 5A), and/or using a different spray head or the same spray head (e.g., spray head 220 of FIG. 1B and FIG. 5B) used to introduce the inert gas into the reaction vessel during the deprotecting step described herein. As a non-limiting example, as depicted in FIG. 1B (and FIG. 5B), the process can include providing (e.g., directing) solvent from a solvent source (not illustrated in FIG. 1B or FIG. 5B) through flow path 218, opening 208, the inner interior space of spray head 220, and out openings 222 into the outer interior space 7 of reaction vessel 4. As also schematically depicted in FIG. 1B (and FIG. 5B), in some embodiments, the spray head 220 can direct (e.g., spray) solvent through openings 222 at an angle (e.g., spray pattern) schematically depicted by dashed lines 224 towards side wall 6 of the reaction vessel 4, which can facilitate washing deprotecting base condensed on the side wall downwardly toward the lower interior portion of reaction vessel 4.

When a washing step in a SPPS process and/or extraction step in a LPPS process is included, the washing solution and/or extraction waste layer (e.g., an aqueous layer including deprotecting base) can then be removed in a draining step, after which a coupling step can be initiated in accordance with known processes.

In some embodiments, the peptide synthesis process (e.g., the solid phase peptide synthesis process and/or the liquid or solution phase peptide synthesis process) can include: deprotecting a first amino acid (e.g., removing a protecting group of a first protected amino acid) to form a deprotected amino acid; coupling a second amino acid to the deprotected amino acid to form a peptide from the first and second amino acids; and repeating the deprotecting and coupling steps to form a peptide comprising the first, second, and successive plurality of amino acids,
wherein the deprotecting and coupling steps take place in a reaction vessel (e.g., in a reaction vessel such as a batch-type reaction vessel 4 described in more detail herein),
wherein one or more of the deprotecting steps employ a deprotecting base in an amount of about 5 vol % or less, based on the total volume of a deprotection reaction mixture (e.g., the deprotection reaction solution), as described in more detail herein, and/or
wherein one or more of the deprotecting steps employ an inert gas purging (e.g., headspace flushing) step to assist in removing (e.g., to remove) evaporated deprotecting base from the interior (e. g., from the headspace) of the reaction vessel, also as described in more detail herein.

In some embodiments, the peptide synthesis process (e.g., the solid phase peptide synthesis process and/or the liquid or solution phase peptide synthesis process) can include: deprotecting a protected peptide (e.g., removing a protecting group of a protected peptide) to form a deprotected peptide; coupling an amino acid to the deprotected peptide to form a second peptide from the deprotected peptide and amino acid; and repeating the deprotecting and coupling steps to form a peptide comprising first, second, and successive plurality of amino acids,
wherein the deprotecting and coupling steps take place in a reaction vessel (e.g., in a reaction vessel such as a batch-type reaction vessel 4 described in more detail herein),
wherein one or more of the deprotecting steps employ a deprotecting base in an amount of about 5 vol % or less, based on the total volume of a deprotection reaction mixture (e.g., the deprotection reaction solution), as described in more detail herein, and/or
wherein one or more of the deprotecting steps employ an inert gas purging (e.g., headspace flushing) step to assist in removing (e.g., to remove) evaporated deprotecting base from the interior (e. g., from the headspace) of the reaction vessel, also as described in more detail herein.

In the SPPS processes including a deprotecting step as described herein, the solid phase peptide synthesis process may not include a washing step between the deprotection step and the coupling step of one or more of the deprotection-coupling cycles of the SPPS process. In other SPPS processes including a deprotecting step as described herein, the solid phase peptide synthesis process may include one or more washing steps between the deprotection step and the coupling step of one or more of the deprotection-coupling cycles of the SPPS process, the washing step using amounts of washing liquid (e.g., solvent) as described in more detail herein (e.g., using solvent in an amount that is about the same as and/or less than the total volume of the deprotection reaction mixture (e.g., the deprotection reaction solution) of the deprotection step, for example, in an amount that is less than or about ½ of the total volume of the deprotection reaction mixture (e.g., the deprotection reaction solution), as another example in an amount that is less than or about ⅓ of the total volume of the deprotection reaction mixture (e.g., the deprotection reaction solution), and as another example in an amount (e.g., total volume) that is 2 times or less of the bed volume of the resin present in the reaction vessel, for example, 1 times or less of the bed volume of the resin present in the reaction vessel). For example, the solid phase peptide synthesis process may omit one or more (e.g., all) washing steps between the deprotection step and the coupling step of one or more of the deprotection-coupling cycles of the SPPS process. As another example, the solid phase peptide synthesis process may include one or more washing steps using reduced amounts of washing liquid (e.g., solvent) as described in more detail herein (e.g., using solvent in an amount that is about the same as and/or is less than the total volume of the deprotection reaction mixture (e.g., the deprotection reaction solution), for example, in an amount that is less than or about ½ of the total volume of the deprotection reaction mixture (e.g., the deprotection reaction solution), as another example in an amount that is less than or about ⅓ of the total volume of the deprotection reaction mixture (e.g., the deprotection reaction solution), and as another example in an amount (e.g., total volume) that is 2 times or less of the bed volume of the resin present in the reaction vessel, for example, 1 times or less of the bed volume of the resin present in the reaction vessel) between the deprotection step and the coupling step of one or more of the deprotection-coupling cycles of the SPPS process.

In the LPPS processes including a deprotecting step as described herein, the liquid phase peptide synthesis process may include one or more extraction steps between the deprotection step and the successive coupling step of one or more deprotection-coupling cycles of the LPPS process, the extraction step(s) using amounts of extraction solvent (e.g., water) as described in more detail herein (e.g., using a total amount of extraction solvent for a single deprotection-coupling cycle that is 2× or less than the total volume of the deprotection reaction mixture (e.g., the deprotection reaction solution) of the deprotection step of that deprotecting-coupling cycle).

The solid phase peptide synthesis and/or liquid phase peptide synthesis process can further include, prior to coupling, activating chemical group(s) on the second amino acid (and successive amino acid(s)) using processes and agents known in the art to prepare the second (and successive) amino acid(s) for coupling with the first (and sequential) amino acid(s).

An amino acid activating agent (amino acid activator) may be used to activate the amino acid (e.g., convert the acid group of the amino acid into an activated form) prior to a coupling step. Any suitable amino acid activating agent may be used. Examples of an amino acid activating agent include without limitation carbodiimides and/or onium salt activating agents. The amino acid activating agent comprises, in some embodiments, a carbodiimide, such as but not limited to N,N′-diisopropylcarbodiimide (DIC), N,N′-dicyclohexylcarbodiimide (DCC), 1-ethyl-3-(3-dimethylaminopropyl)carbodiimide (EDC), and the like and combinations thereof. In certain embodiments, the amino acid activating agent comprises an onium activating agent, such as but not limited to benzotriazol-1-yloxytripyrrolidinophosphonium hexafluorophosphate (PyBOP), O-(benzotriazol-1-yl)-N,N,N′,N′-tetramethyluronium hexafluorophosphate (HBTU), 2-(7-aza-1H-benzotriazole-1-yl)-1,1,3,3-tetramethyluronium hexafluorophosphate (HATU), 1-[(1-(cyano-2-ethoxy-2-oxoethylideneaminooxy) dimethylaminomorpholino)] uronium hexafluorophosphate (COMU), and the like and combinations thereof.

An amino acid activating agent additive (amino acid activator additive) may also be used to activate the amino acid prior to a coupling step. Any suitable amino acid activator additive may be used. Examples of amino acid activator additives include without limitation benzotriazole additives, such as 1-hydroxybenzotriazole (HOBt), 1-hydroxy-7-azabenzotriazole (HOAt), and 6-chloro-1-hydroxybenzotriazole (6-Cl-HOBt); ethyl (hydroxyimino)cyanoacetate (Oxyma); 1-hydroxy-2,5-pyrrolidinedione (NHS), and the like and combinations thereof.

Still further, in exemplary embodiments, the solid phase peptide synthesis and/or liquid phase peptide synthesis process can include applying microwave energy during one or more of the solid phase peptide synthesis and/or liquid phase peptide synthesis steps, for example, during the deprotecting and/or coupling steps.

In exemplary embodiments, the solid phase peptide synthesis process can further include cleaving the peptide from the solid phase resin after the deprotecting, optional washing, and/or coupling steps.

In exemplary embodiments, the liquid phase peptide synthesis process can further include cleaving the peptide from support and/or carrier and/or protecting group materials (e.g., from a soluble tag) after the deprotecting, extraction, and/or coupling steps.

The skilled artisan will understand how to join or couple amino acids to form a chain. Processes and agents for cleaving a peptide from a solid phase resin and/or LPPS support and/or carrier and/or protecting group materials (e.g., from a soluble tag) are also well known in the art. Accordingly, a detailed discussion of processes known in the art for joining amino acids to form a peptide and/or cleaving a peptide from a solid phase resin and/or LPPS support and/or carrier and/or protecting group materials (e.g., from a soluble tag) is not provided.

The scale of solid phase peptide synthesis and/or liquid phase peptide synthesis including deprotection processes disclosed herein is not limited and may include, for example, research and/or production (e.g., large) scale solid phase peptide synthesis and/or liquid phase peptide synthesis.

In exemplary embodiments, the deprotection processes disclosed herein (e.g., including one or more deprotecting steps as described herein using a deprotecting base in an amount from greater than zero to about 5 vol %, based on the total volume of the deprotection reaction mixture (e.g., the deprotection reaction solution) and/or using an inert gas to flush volatized deprotecting base from the reaction vessel (e.g., from the head space of the interior of the reaction vessel), also as described in more detail herein) may be used on large scale systems that incorporate the ability to heat the reaction solution (e.g., a large scale Liberty PRO system available from CEM Corporation at 25 mmol, which is around about 125 grams of resin at 0.2 mmol/g.) In such embodiments, the amount of base (e.g., pyrrolidine) may be used in very low concentrations much less than typically used—amount of 2.5% vol (standard is 20%), which can facilitate reducing and/or eliminating washing. Also in some of these embodiments, deprotection conditions may be conducted for variable times and temperatures to facilitate more complete deprotection and base removal to a satisfactory removal. One example of this is 10 minutes at 90° C.

In other exemplary embodiments, the deprotection processes disclosed herein may include:
a process for deprotecting a protected amino acid during solid phase peptide synthesis (SPPS) and/or liquid phase peptide synthesis (LPPS) including deprotecting steps and coupling steps, the deprotecting process comprising:
removing a protecting group of a protected amino acid and/or a protected peptide in a reaction vessel with a deprotecting base, wherein at least a portion of the deprotecting base evaporates into an upper interior portion of the reaction vessel during the removing step; and
directing an inert gas through the reaction vessel to assist in removing evaporated deprotecting base from the interior of the reaction vessel during the step of removing the protecting group,
wherein the deprotecting process may be conducted under one or more of the following conditions:
(a) in a SPPS process, the deprotecting process can use (e.g., a protected amino acid can be attached to) a solid resin support having a resin substitution of less than or about 0.35 mmol/g, for example, less than or about 0.30 mmol/g, for example 0.10 mmol/g to 0.35 mmol/g, for example 0.15 mmol/g to 0.35 mmol/g, for example 0.20 mmol/g to 0.35 mmol/g, for example 0.10 mmol/g to 0.34 mmol/g, for example 0.15 mmol/g to 0.34 mmol/g, for example 0.20 mmol/g to 0.34 mmol/g, for example 0.20 mmol/g to 0.33 mmol/g; and/or
(b) the deprotecting process may use a deprotecting base (e.g., pyrrolidine) in an amount greater than zero to about 5 vol %, for example greater than zero to about or less than 3.5 vol %, based on the total volume (100 vol %) of a deprotection reaction mixture (e.g., a deprotection reaction solution) in the reaction vessel as defined herein; and/or
(c) the deprotecting process may be conducted at a temperature above 30° C., for example, above 50° C., as another example above 70° C., as another example at a temperature from 30° C. to 120° C.; and/or
(d) the directing step may include directing the inert gas into the reaction vessel through a first opening and out of a second opening located in an upper portion of the reaction vessel to assist in removing evaporated deprotecting base from the interior of the reaction vessel (e.g., to purge, vent, etc. evaporated deprotecting base from the headspace); and/or
(e) in a SPPS process, the deprotecting process optionally can include washing the interior of the reaction vessel after the deprotecting step using a total volume of solvent that is 2 times or less of the bed volume of the resin present in the reaction vessel, for example, 1 times or less of the bed volume of the resin present in the reaction vessel; and/or
(f) the total time for the deprotection reaction (e.g., the total time for a deprotection reaction of one deprotection-coupling cycle of a SPPS process) may be within 1.5 hours or less, for example, from about 30 seconds to about an hour, for example from about 30 seconds to about 10 minutes, and as another example from about 10 minutes to about an hour; and/or
(g) the boiling point of the deprotecting base is less than 107° C.; and/or
(h) a difference between the deprotection reaction temperature and boiling point of the deprotecting base may be less than or about 50° C., for example less than or about 25° C., for example less than or about 15° C., for example the difference between the deprotection reaction temperature and the boiling point of the deprotecting base may range from 15° C. to 50° C.; and/or
(i) in a LPPS process, a total amount of extraction solvent for a single deprotection-coupling cycle may be 2× or less than the total volume of the deprotection reaction mixture (e.g., the deprotection reaction solution) of the deprotection step; and/or
(j) the deprotection may be performed at a temperature that is no less than 35° C. below the boiling point of the deprotecting base and/or for a time of 1.5 hours or less.

In exemplary embodiments, the process includes: heating the protected amino acid and the deprotecting base during the step of removing the protecting group from the protected amino acid; the deprotecting base is pyrrolidine; the protected amino acid is attached directly or indirectly to a solid PEG-PS (polyethylene glycol-polystyrene) resin support, wherein the resin can have a resin substitution of from 0.2 mmol/g to 0.3 mmol/g, for example from 0.20 mmol/g to 0.25 mmol/g; the protecting group of the protected amino acid is a 9-fluorenylmethyloxycarbonyl (Fmoc) protecting group; and the heating step is conducted at a temperature of about 60° C. or higher.

Other exemplary embodiments of the present disclosure may include but are not limited to processes for deprotecting a protected peptide during liquid phase peptide synthesis including deprotecting steps and coupling steps, wherein the deprotecting process includes removing a protecting group of a protected peptide in the liquid phase, wherein the C-terminus of the protected peptide is protected, in a reaction vessel with a deprotecting base at a temperature that is no less than 35° C. below the boiling point of the deprotecting base and for a time of 1.5 hours or less to provide a deprotected peptide. The deprotecting base is present in the reaction vessel in an amount greater than zero to about 5 vol %, based on the total volume (100 vol %) of a deprotection reaction mixture in the reaction vessel, and a majority of the deprotecting base evaporates into an upper interior portion of the reaction vessel during the removing of the protecting group of the protected peptide. The process may further include directing an inert gas through the reaction vessel to assist in the removal of evaporated deprotecting base from the interior of the reaction vessel during the removing the protecting group of the protected peptide; and extracting residual deprotecting base from the deprotection reaction mixture after removing the protecting group and before a successive coupling step of one or more deprotection-coupling cycles of the liquid phase peptide synthesis using a total volume of extraction solvent that is 2× or less than the total volume of the deprotection reaction mixture of that deprotecting-coupling cycle. In some embodiments, the directing step may include directing inert gas into an upper interior portion of the reaction vessel through a first opening located in an upper portion of the reaction vessel; and venting inert gas and evaporated deprotecting base from the upper interior portion of the reaction vessel through a second opening located in the upper portion of the reaction vessel.

Other exemplary embodiments of the present disclosure may include but are not limited to processes for deprotecting a protected peptide during liquid phase peptide synthesis including deprotecting steps and coupling steps, wherein the deprotecting step includes removing a protecting group of a protected peptide in the liquid phase, wherein the C-terminus of the protected peptide is protected, in a reaction vessel with a deprotecting base at a temperature that is no less than 35° C. below the boiling point of the deprotecting base and for a time of 1.5 hours or less to provide a deprotected peptide. The deprotecting base is present in the reaction vessel in an amount greater than zero to about 5 vol %, based on the total volume (100 vol %) of a deprotection reaction mixture in the reaction vessel, and a majority of the deprotecting base evaporates into an upper interior portion of the reaction vessel during the removing of the protecting group of the protected peptide. The deprotecting step also includes directing inert gas into an upper interior portion of the reaction vessel through a first opening located in an upper portion of the reaction vessel; and venting inert gas and evaporated deprotecting base from the upper interior portion of the reaction vessel through a second opening located in the upper portion of the reaction vessel. In some embodiments, the process may further include extracting residual deprotecting base from the deprotection reaction mixture after removing the protecting group and before a successive coupling step of one or more deprotection-coupling cycles of the liquid phase peptide synthesis using a total volume of extraction solvent that is 2× or less than the total volume of the deprotection reaction mixture of that deprotecting-coupling cycle.

Other exemplary embodiments of the present disclosure may include but are not limited to processes for liquid phase peptide synthesis (LPPS), including deprotecting a protected peptide in the liquid phase, wherein the C-terminus of the protected peptide is protected, in a reaction vessel to provide a deprotected peptide. The deprotecting includes removing a protecting group of the protected peptide with a deprotecting base at a temperature that is no less than 35° C. below the boiling point of the deprotecting base and for a time of 1.5 hours or less, wherein the deprotecting base is present in the reaction vessel in an amount great than zero to about 5 vol %, based on the total volume (100 vol %) of a deprotection reaction mixture in the reaction vessel, and wherein a majority of the deprotecting base evaporates into an upper interior portion of the reaction vessel during the removing of the protecting group; and directing an inert gas through the reaction vessel to remove evaporated deprotecting base from the interior of the reaction vessel during the removing of the protecting group. The LPPS may further include extracting residual deprotecting base from the deprotection reaction mixture after removing the protecting group and before a successive coupling step of one or more deprotection-coupling cycles of the liquid phase peptide synthesis using a total volume of extraction solvent that is 2× or less than the total volume of the deprotection reaction mixture of that deprotecting-coupling cycle; coupling an amino acid to the deprotected peptide to form a peptide from the deprotected peptide and the amino acid; and repeating the deprotecting, extracting, and coupling to form a peptide further comprising one or more successive amino acids. In some embodiments, the directing step may include directing inert gas into an upper interior portion of the reaction vessel through a first opening located in an upper portion of the reaction vessel; and venting inert gas and evaporated deprotecting base from the upper interior portion of the reaction vessel through a second opening located in the upper portion of the reaction vessel.

Other exemplary embodiments of the present disclosure may include but are not limited to processes for liquid phase peptide synthesis (LPPS), including deprotecting a protected peptide in the liquid phase, wherein the C-terminus of the protected peptide is protected, in a reaction vessel to provide a deprotected peptide. The deprotecting may include removing a protecting group of the protected peptide with a deprotecting base at a temperature that is no less than 35° C. below the boiling point of the deprotecting base and for a time of 1.5 hours or less, wherein the deprotecting base is present in the reaction vessel in an amount great than zero to about 5 vol %, based on the total volume (100 vol %) of a deprotection reaction mixture in the reaction vessel, and wherein a majority of the deprotecting base evaporates into an upper interior portion of the reaction vessel during the removing of the protecting group; and directing inert gas into an upper interior portion of the reaction vessel through a first opening located in an upper portion of the reaction vessel; and venting inert gas and evaporated deprotecting base from the upper interior portion of the reaction vessel through a second opening located in the upper portion of the reaction vessel to remove evaporated deprotecting base from the reaction vessel during the removing of the protecting group. The LPPS process further includes coupling an amino acid to the deprotected peptide to form a peptide from the deprotected peptide and the amino acid; and repeating the deprotecting and coupling to form a peptide further comprising one or more successive amino acids. In some embodiments, the LPPS process may further include extracting residual deprotecting base from the deprotection reaction mixture after removing the protecting group and before a successive coupling step of one or more deprotection-coupling cycles of the liquid phase peptide synthesis using a total volume of extraction solvent that is 2× or less than the total volume of the deprotection reaction mixture of that deprotecting-coupling cycle.

Other exemplary embodiments of the present disclosure may include but are not limited to processes for deprotecting a protected amino acid during solid phase peptide synthesis (SPPS) including deprotecting steps and coupling steps, wherein the deprotecting process includes removing a protecting group of a protected amino acid attached to a solid resin support in a reaction vessel having a size of at least 3 liters with a deprotecting base to provide a deprotected amino acid. The deprotecting base is present in the reaction vessel in an amount greater than zero to about 5 vol %, based on the total volume (100 vol %) of a deprotection reaction mixture in the reaction vessel, and at least a portion of the deprotecting base evaporates into an upper interior portion of the reaction vessel during the removing of the protecting group of the protected amino acid. The deprotecting process further includes directing an inert gas having a pressure of about 50 psi to about 95 psi through the reaction vessel to remove evaporated deprotecting base from the interior of the reaction vessel during the removing the protecting group of the protected amino acid.

Other exemplary embodiments of the present disclosure may include but are not limited to processes for solid phase peptide synthesis having a synthesis scale of about 25 mmol or higher. The processes may include deprotecting a first protected amino acid to provide a deprotected amino acid; and coupling a second amino acid to the deprotected amino acid to form a peptide from the first and second amino acids. The deprotecting may include removing a protecting group of the protected amino acid in a reaction vessel having a size of at least 3 liters with a deprotecting base, wherein the deprotecting base is present in the reaction vessel in an amount great than zero to about 5 vol %, based on the total volume (100 vol %) of a deprotection reaction mixture in the reaction vessel, and wherein at least a portion of the deprotecting base evaporates into an upper interior portion of the reaction vessel during the removing of the protecting group; and directing an inert gas having a pressure of about 50 psi to about 95 psi through the reaction vessel to remove evaporated deprotecting base from the interior of the reaction vessel during the removing of the protecting group.

The present disclosure also relates to a system for solid phase peptide synthesis and/or liquid phase peptide synthesis. FIG. 2A (and FIG. 6A) is a schematic flow diagram depicting selected portions of an exemplary solid phase peptide synthesis system and/or liquid phase peptide synthesis system in accordance with embodiments of the present disclosure. FIG. 2B (and FIG. 6B) is a schematic flow diagram depicting selected portions of another exemplary solid phase peptide synthesis system and/or liquid phase peptide synthesis system in accordance with other embodiments of the present disclosure

Generally, the elements illustrated in both FIG. 1A and FIG. 2A (and FIGS. 5A and 6A) will carry the same reference numerals. Similarly, generally the elements illustrated in both FIG. 1B and FIG. 2B (and FIGS. 5B and 6B) will carry the same reference numerals. Also, except where indicated otherwise, elements illustrated in both FIG. 2A and FIG. 2B (and FIGS. 6A and 6B) will carry the same reference numerals.

The peptide synthesis system of FIGS. 2A and 2B (and FIGS. 6A and 6B) is designated generally as 2. The peptide synthesis systems 2 of FIGS. 2A and 2B (and FIGS. 6A and 6B) are suitable for use in SPPS processes and/or LPPS processes as described herein. Peptide synthesis system 2 includes reaction vessel 4 as discussed herein. Peptide synthesis system 2 also includes a plurality of reagent containers located in a position upstream of reaction vessel 4 in fluid communication with reaction vessel 4.

For example, as depicted in FIGS. 2A and 2B (and FIGS. 6A and 6B), system 2 can include a plurality of solid support and/or support suitable for LPPS processes (e.g., soluble tag) containers 50a, 50b, and 50c in fluid communication with a flow path 52 fluidly connecting the solid support and/or support for LPPS (e.g., soluble tag) containers and reaction vessel 4 for delivering a solid support (e.g., a solid resin having a protected amino acid linked thereto) and/or support for LPPS (e.g., soluble tag having a protected amino acid linked thereto) from the solid support and/or support for LPPS (e.g., soluble tag) container(s) to reaction vessel 4. Flow paths 51a, 51b, and 51c fluidly connect solid support and/or support for LPPS (e.g., soluble tag) containers 50a, 50b, and 50c, respectively, and flow path 52.

In certain embodiments, flow path 52 can be in direct fluid communication with reaction vessel 4, e.g., via opening 12 or 204 of FIG. 1A or 1B (and FIG. 5A or 5B), respectively. In certain embodiments, as depicted schematically in FIG. 2A (and FIG. 6A), the system can include a rotary valve 140 that can rotate among multiple positions (e.g., two positions) to fluidly connect reaction vessel 4 through opening 12, flow path 22, and flow path 52 with solid support and/or support for LPPS (e.g., a soluble tag) containers 50a, 50b, and 50c. In certain other embodiments, as depicted schematically in FIG. 2B (and FIG. 6B), rotary valve 140 can rotate among multiple positions (e.g., two positions, or more) to fluidly connect reaction vessel 4 through opening 204, flow path 214, and flow path 52 with solid support and/or support for LPPS (e.g., a soluble tag) containers 50a, 50b, and 50c. During the deprotecting step described herein, as schematically depicted in FIGS. 2A and 2B (and FIGS. 6A and 6B), rotary valve 140 may be closed with respect to flow path 52 (and flow path 152).

As another example, in certain embodiments, as depicted schematically in FIGS. 2A and 2B (and FIGS. 6A and 6B), system 2 can include a plurality of amino acid containers 60a, 60b, and 60c in fluid communication with flow path 62 (FIG. 2A and FIG. 6A) or flow path 212 (FIG. 2B and FIG. 6B) fluidly connecting the amino acid containers and reaction vessel 4 for delivering protected amino acid from the amino acid container(s) to reaction vessel 4. Flow paths 61a, 61b, and 61c fluidly connect amino acid containers 60a, 60b, and 60c, respectively, with flow path 62 (FIG. 2A and FIG. 6A) or flow path 212 (FIG. 2B and FIG. 6B). In some embodiments, flow path 62 and/or flow path 212 can be in direct fluid communication with reaction vessel 4, e.g., via opening 10 or 202 of FIG. 1A or 1B (and FIGS. 5A and 5B), respectively. In some embodiments, the system can include one or more additional valves and/or flow paths, as schematically depicted in FIGS. 2A and 2B (and FIGS. 6A and 6B) and as described in more detail below. During the deprotecting step described herein, opening 10 or 202 may be closed with respect to flow path 62 or flow path 212 of FIGS. 2A and 2B (and FIGS. 6A and 6B), respectively.

As another example, as schematically depicted in FIGS. 2A and 2B (and FIGS. 6A and 6B), system 2 can include a deprotecting base container 70 in fluid communication with a flow path 72 fluidly connecting the deprotecting base container and reaction vessel 4 for delivering deprotecting base (for example, as part of a deprotection solution as described herein) from the deprotecting base container to reaction vessel 4. In some embodiments, flow path 72 can be in direct fluid communication with reaction vessel 4, e.g., via opening 16 of FIGS. 1A and 1B (and FIGS. 5A and 5B). In some embodiments, the system can include one or more additional valves and/or flow paths, as schematically depicted in FIGS. 2A and 2B (and FIGS. 6A and 6B) and as described in more detail below. During the deprotecting step described herein, opening 16 may be closed with respect to flow path 72 of FIGS. 2A and 2B (and FIGS. 6A and 6B), respectively.

As yet another example, as schematically depicted in FIGS. 2A and 2B (and FIGS. 6A and 6B), system 2 can include a solvent container 80 in fluid communication with a flow path 82 fluidly connecting the solvent container and reaction vessel 4 for delivering solvent from the solvent container to reaction vessel 4. In some embodiments, flow path 82 can be in direct fluid communication with reaction vessel 4, e.g., via opening 10 or 208 of FIG. 1A or 1B (and FIG. 5A or 5B), respectively. In some embodiments, the system can include one or more additional valves and/or flow paths, as schematically depicted in FIGS. 2A and 2B (and FIGS. 6A and 6B) and as described in more detail below. FIG. 2B (and FIG. 6B) also schematically depicts embodiments wherein solvent can be introduced into reaction vessel 4 using at least a portion of the same flow path used to introduce an inert gas, e.g., via flow path 218, opening 208, spray head 220 and plurality of openings 222, as described in more detail herein. During the deprotecting step described herein, opening 10 or 208 may be closed with respect to flow path 82 of FIGS. 2A and 2B (and FIGS. 6A and 6B), respectively.

As yet another example, as schematically depicted in FIGS. 6A and 6B, for LPPS processes, the system 2 can include additional solvent containers (e.g., extraction solvent containers), such as solvent container 82a, that can be in fluid communication with a flow path 82a fluidly connecting the extraction solvent container and reaction vessel 4 for delivering extraction solvent from the solvent container to reaction vessel 4. In some embodiments, flow path 82a can be in direct fluid communication with reaction vessel 4, e.g., via opening 10 or 208 of FIG. 1A or 1B (and FIG. 5A or 5B), respectively. In some embodiments, the system can include one or more additional valves and/or flow paths, as schematically depicted in FIGS. 6A and 6B and as described in more detail below. FIG. 6B also schematically depicts embodiments wherein solvent can be introduced into reaction vessel 4 using at least a portion of the same flow path used to introduce an inert gas, e.g., via flow path 218, opening 208, spray head 220 and plurality of openings 222, as described in more detail herein. During the deprotecting step described herein, opening 10 or 208 may be closed with respect to flow path 82a of FIGS. 6A and 6B, respectively.

As yet another example, as schematically depicted in FIGS. 2A and 2B (and FIGS. 6A and 6B), system 2 can include an additional reagent container 90, which can be for example an activating agent container, in fluid communication with a flow path 92 (FIG. 2A and FIG. 6A) or flow path 210 (FIG. 2B and FIG. 6B) fluidly connecting the additional reagent container and reaction vessel 4 for delivering an additional reagent such as an activating agent from the additional reagent container to reaction vessel 4. In some embodiments, flow path 92 or 210 can be in direct fluid communication with reaction vessel 4, e.g., via opening 10 or 200 of FIGS. 1A and 1B (and FIGS. 5A and 5B), respectively. In some embodiments, the system can include one or more additional valves and/or flow paths, as schematically depicted in FIGS. 2A and 2B (and FIGS. 6A and 6B) and as described in more detail below. During the deprotecting step described herein, opening 10 or 200 may be closed with respect to flow path 92 or flow path 210 of FIGS. 2A and 2B (and FIGS. 6A and 6B), respectively.

The skilled artisan will appreciate that the number of reaction vessels, solid support containers, amino acid containers, deprotecting base containers, solvent containers, and/or other reagent containers and associated flow paths, as well as the manner in which these elements are connected, can vary and is not limited to the depiction thereof in FIG. 2A and FIG. 2B (and FIGS. 6A and 6B). The skilled artisan will also appreciate that the system can include other containers such as a container for an extraction solvent and associated flow paths (e.g., for delivery of the extraction solvent to the reaction vessel); associated valves, pathways, containers, etc. for removal of extraction waste from a reaction vessel and/or for disposal thereof, etc. The skilled artisan will also understand that the peptide synthesis system can include various subsystems associated with the aforementioned containers, flow paths, and/or reaction vessel(s), including for example flow paths, valves, filters, gauges, monitors, controllers, etc., to direct the flow of materials into and/or out of containers and/or the reaction vessel(s) at appropriate stages of the solid phase peptide synthesis process and/or liquid phase peptide synthesis process. Such subsystems are well known in the art and will not be described in detail herein.

System 2 is also associated with a heating source (not shown) such as a microwave source, and associated elements such as microwave guides and/or microwave cavities, for heating reaction vessel 4, as described herein. Heating sources, including microwave heating sources, and associated elements such as microwave guides and/or microwave cavities, and the use thereof in solid phase peptide synthesis and/or liquid phase peptide synthesis processes and systems, are also well known in the art and are not described in more detail herein.

System 2 is schematically depicted as operating in an amino acid deprotecting step and/or peptide deprotecting step of a solid phase peptide synthesis and/or liquid phase peptide synthesis, such as described herein with reference to FIG. 1A and FIG. 1B (and FIGS. 5A and 5B). In this operational state, reactants including a protected amino acid and/or a protected peptide and a deprotecting base have already been delivered to reaction vessel 4 (and/or are already present in reaction vessel 4). The reaction vessel also may include a coupling solution from a preceding coupling step, which together with the deprotection solution forms the deprotection reaction mixture (e.g., the deprotection reaction solution), all also as described herein.

The protected amino acid and/or a protected peptide can be attached to a solid support and/or a suitable support for LPPS (e.g., a soluble tag). In some embodiments, the protected amino acid and/or protected peptide can be directly attached to the solid support and/or LPPS support (e.g., a soluble tag) (e.g., can be a protected amino acid and/or a protected peptide directly attached to a solid support and/or LPPS support (e.g., a soluble tag) delivered to reaction vessel 4 via flow path 52 from one or more of solid support and/or LPPS support (e.g., a soluble tag) containers 50a, 50b, and 50c). In some embodiments (e.g., after a preceding coupling step), the protected amino acid and/or protected peptide can be directly or indirectly attached to a solid support and/or LPPS support (e.g., a soluble tag) (e.g., can be attached to another amino acid or to a growing peptide chain, wherein the other amino acid or peptide is attached to the solid support and/or LPPS support (e.g., a soluble tag)).

System 2 further includes an inert gas source 100 located in a position upstream of reaction vessel 4 in fluid communication with reaction vessel 4. In certain embodiments, as depicted schematically in FIGS. 2A and 2B (and FIGS. 6A and 6B), a flow path 102 fluidly connects inert gas source 100 and reaction vessel 4 and delivers inert gas supplied from inert gas source 100 into the upper interior portion of reaction vessel 4 via opening 10 (FIG. 1A and FIG. 5A) or opening 208 (FIG. 1B and FIG. 5B) described herein. In certain embodiments, system 2 includes a valve 104 in fluid communication with flow path 102 located between inert gas source 100 and opening 10 (FIG. 1A and FIG. 5A) or opening 208 (FIG. 1B and FIG. 5B), wherein valve 104 has an open position and a closed position relative to flow path 102.

Valve 104 is shown in an open position in FIG. 2A and FIG. 2B (and FIGS. 6A and 6B) relative to flow path 102. In the open position, valve 104 allows flow of pressurized inert gas from inert gas source 100 through flow path 102, flow path 20 (FIG. 1A and FIG. 5A) or flow path 218 (FIG. 1B and FIG. 5B), and opening 10 (FIG. 1A and FIG. 5A) or opening 208 and spray head 220 (FIG. 1B and FIG. 5B), and into the interior upper portion of reaction vessel 4 (e.g., into the reaction vessel headspace). In this manner, the system can continuously and/or intermittently direct an overhead source of pressurized inert gas into the upper portion of the interior of the reaction vessel during the heating/deprotecting step to purge volatized deprotecting base out of the reactor headspace, in accordance with the process described herein.

In contrast, when valve 104 is in a closed position relative to flow path 102, valve 104 can prevent the flow of pressurized inert gas from the inert gas source through flow path 102, flow path 20 (FIG. 1A and FIG. 5A) or flow path 218 (FIG. 1B and FIG. 5B), and opening 10 (FIG. 1A and FIG. 5A) or opening 208 and spray head 220 (FIG. 1B and FIG. 5B).

The system can also include a flow path 106 fluidly connecting inert gas source 100 and opening 16 in the lower portion of reaction vessel 4. The system can also include a valve 108 in fluid communication with flow path 106 located between inert gas source 100 and opening 16, wherein valve 108 has an open position and a closed position relative to flow path 106.

When in the open position relative to flow path 106 (such as illustrated in FIGS. 2A and 2B and FIGS. 6A and 6B), valve 108 allows flow of pressurized gas from inert gas source 100 through flow path 106, flow path 26, and opening 16 into the interior lower portion of reaction vessel 4. As described herein, in the deprotecting process (e.g., when valve 108 is optionally open), in this manner pressurized inert gas can be directed into the lower portion of the reaction vessel to agitate (e.g., stir, bubble) the reactants and/or flush evaporated deprotecting base from the headspace of the reaction vessel. The closed position of second valve 108 relative to flow path 106 prevents flow of pressurized inert gas from inert gas source 100 through flow path 106, flow path 26, and opening 16 into the interior lower portion of reaction vessel 4.

In certain embodiments, flow paths 102 and 106 can be fluidly connected via a pressure regulator, designated in FIG. 2A (and FIG. 6A) and FIG. 2B (and FIG. 6B) as 110, to a single inert gas source 100. Alternatively, flow paths 102 and 106 can fluidly connect the reaction vessel to at least two different inert gas sources.

When present, pressure regulator 110 can be located in a downstream position from inert gas source 100 and an upstream position from valves 104 and 108. In these embodiments, pressure source 100 directs inert gas to pressure regulator 110, which supplies pressurized inert gas to flow path 102 having a higher pressure (the “high pressure” inert gas) than inert gas supplied to flow path 106 (the “low pressure” inert gas). For example, without being limited thereto, pressure regulator 110 can supply “high pressure” inert gas having a pressure of about 1 psi to about 25 psi to flow path 102. Pressure regulator 110 can also supply “low pressure” inert gas having a pressure that is less than the pressure of the “high pressure” inert gas supplied to flow path 102.

Pressure regulators are also well known in the art and the skilled artisan will understand how to use the same in system 2 to provide a high pressure inert gas and a low pressure inert gas as discussed herein.

As also schematically depicted in FIGS. 2A and 2B and FIGS. 6A and 6B (and FIGS. 1A and 1B and FIGS. 5A and 5B), in exemplary embodiments, the system can include flow path 24 (FIGS. 1A/5A and 2A/6A) or flow path 216 (FIGS. 1B/5B and 2B/6B) located in a downstream position from opening 14 (FIG. 1A and FIG. 5A) or opening 206 (FIG. 1B and FIG. 5B) of reaction vessel 4. Flow paths 24 and 216 function as a gaseous waste flow path to allow the purging of gaseous waste (e.g., volatized deprotecting base, inert gas, etc.) during the deprotecting process described herein from the upper interior portion (headspace) of reaction vessel 4.

In certain embodiments, the system includes a valve 120 in fluid communication with flow path 24 or flow path 216. Valve 120 has an open position and a closed position relative to flow path 24 or flow path 216. The open position of valve 120 allows flow of gas from the upper interior portion of reaction vessel 4 through opening 14 or opening 206 and flow path 24 or flow path 216 to a waste recovery zone, such as a vent and/or waste container (not shown) to permit purging/venting of gas from reaction vessel 4. The closed position of valve 120 prevents flow of gas out of the upper interior portion of the reaction vessel through opening 14 or opening 206.

FIGS. 2A and 2B (and FIGS. 6A and 6B) illustrate certain embodiments of the system in an operational state wherein both valve 104 and valve 120 are in an open position with respect to flow path 102, flow path 20 or 218, and flow path 24 or flow path 216, respectively. This corresponds to the positions that would be used during the (heated) deprotecting process described herein. The simultaneously open positions of valve 104 and valve 120 relative to flow path 102, flow path 20 or 218, and flow path 24 or flow path 216, respectively, allow an overhead supply of pressurized inert gas to flow continuously and/or intermittently through reaction vessel 4 (for example, flow into vessel 4 through opening 10 in the vessel headspace and out of vessel 4 through a separate opening 14 or as another example flow into vessel 4 through opening 208, spray head 220 and openings 222 toward side wall 6 and into the vessel headspace and out of vessel 4 through a separate opening 206) to purge (flush) volatized reactants present in the headspace during the (heated) deprotecting step.

In certain embodiments, system 2 can include a valve 122 in series with valve 104 and a flow path 124 positioned (disposed) between and fluidly connecting valve 104 and valve 122. Valve 122 is in fluid communication with flow path 102 and has an open position and a closed position relative to flow path 102. When valve 122 is present and valve 122 and valve 104 are in an open position relative to flow path 102, such as depicted in FIG. 2A (and FIG. 6A) and FIG. 2B (and FIG. 6B), inert gas source 100, pressure regulator 110, flow path 102, valve 104, flow path 124, valve 122, flow path 20 (FIG. 2A and FIG. 6A) or flow path 218 (FIG. 2B and FIG. 6B), opening 10 (FIG. 1A/5A/2A/6A) or opening 208 and spray head 220 (FIG. 1B/5B/2B/6B), and reaction vessel 4 can be fluidly connected (in fluid communication).

In certain embodiments, valve 104 can be a rotary valve which can rotate between multiple positions to fluidly connect one flow path selected from a plurality of flow paths with reaction vessel 4. As a non-limiting example, FIG. 2A depicts rotary valve 104 capable of rotating between four positions to fluidly communicate with flow path 102, 62, 82, or 92, depending on the open or closed position of the valve. As another non-limiting example, FIG. 6A depicts rotary valve 104 capable of rotating between five positions to fluidly communicate with flow path 102, 62, 82, 82a, or 92, depending on the open or closed position of the valve. For example, FIG. 2A schematically depicts rotary valve 104 in an open position with respect to flow path 102 but in a closed position with respect to flow paths 62, 82, and 92. For example, FIG. 6A schematically depicts rotary valve 104 in an open position with respect to flow path 102 but in a closed position with respect to flow paths 62, 82, 82a and 92. As another non-limiting example, FIG. 2B depicts rotary valve 104 capable of rotating between two positions to fluidly communicate with flow path 102 or 82, depending on the open or closed position of the valve. As another non-limiting example, FIG. 6B depicts rotary valve 104 capable of rotating between three positions to fluidly communicate with flow path 102, 82, or 82a, depending on the open or closed position of the valve. For example, FIG. 2B schematically depicts rotary valve 104 in an open position with respect to flow path 102 but in a closed position with respect to flow path 82. For example, FIG. 6B schematically depicts rotary valve 104 in an open position with respect to flow path 102 but in a closed position with respect to flow paths 82 and 82a. The open/closed positions of rotary valve 104 relative to the different flow paths can be selected depending on the stage of the peptide synthesis process and the reactants, etc. to be delivered to reaction vessel 4. Rotary valve 104 (and other valves discussed herein) can be operated as known in the art.

Other valves of the system can also be rotary valves. For example, as noted herein, in certain embodiments, system 2 can include a flow path 124 positioned (disposed) between and fluidly connecting valve 104 and valve 122. In this embodiment, as depicted schematically in FIG. 2A and FIG. 2B (and FIGS. 6A and 6B), valve 122 in series with valve 104 can rotate among multiple positions (e.g., valve 122 between two positions in FIGS. 2A and 2B (and FIGS. 6A and 6B) and valve 104 between four positions as schematically depicted in FIG. 2A, between five positions as schematically depicted in FIG. 6A, between two positions as schematically depicted in FIG. 2B, and between three positions as schematically depicted in FIG. 6B) to fluidly connect, for example, inert gas source 100 and reaction vessel 4 via flow path 102, flow path 124, flow path 20 or flow path 218, and opening 10 or opening 208. Alternatively, as shown in FIG. 2A, valve 122 in series with valve 104 can rotate among multiple positions (e.g., two positions and four positions, respectively) to fluidly connect, for example, one or more amino acid containers 60a, 60b, and 60c and reaction vessel 4 via flow path 62, flow path 124, flow path 20, and opening 10; solvent container 80 and reaction vessel 4 via flow path 82, flow path 124, flow path 20, and opening 10; or reagent container 90 and reaction vessel 4 via flow path 92, flow path 124, flow path 20, and opening 10. Alternatively, as shown in FIG. 6A, valve 122 in series with valve 104 can rotate among multiple positions (e.g., two positions and five positions, respectively) to fluidly connect, for example, one or more amino acid containers 60a, 60b, and 60c and reaction vessel 4 via flow path 62, flow path 124, flow path 20, and opening 10; solvent container 80 and reaction vessel 4 via flow path 82, flow path 124, flow path 20, and opening 10; extraction solvent container 80a and reaction vessel 4 via flow path 82a, flow path 124, flow path 20, and opening 10; or reagent container 90 and reaction vessel 4 via flow path 92, flow path 124, flow path 20, and opening 10. Also in some embodiments, as shown in FIG. 2B, valve 122 in series with valve 104 can rotate among multiple positions (e.g., two positions) to fluidly connect, for example, solvent container 80 and reaction vessel 4 via flow path 82, flow path 124, flow path 218, and opening 208 and spray head 222. Also in some embodiments, as shown in FIG. 6B, valve 122 in series with valve 104 can rotate among multiple positions (e.g., two positions and three positions, respectively) to fluidly connect, for example, solvent container 80 and reaction vessel 4 via flow path 82, flow path 124, flow path 218, and opening 208 and spray head 222; or extraction solvent container 80a and reaction vessel 4 via flow path 82a, flow path 124, flow path 218, and opening 208 and spray head 222.

As another example, in certain embodiments, as depicted schematically in FIG. 2A and FIG. 2B (and FIGS. 6A and 6B), valve 108 can be a rotary valve rotating among multiple positions (e.g., three positions) to fluidly connect reaction vessel 4 through opening 16, flow path 26, and flow path 72, flow path 106 or a flow path 132 with deprotecting base container 70, inert gas source 100, or a waste container 130, respectively, depending on the position of valve 108. For example, FIG. 2A and FIG. 2B (and FIGS. 6A and 6B) schematically depict rotary valve 108 in an open position with respect to flow path 106 but in a closed position with respect to flow paths 72 and 132. This can be the position of rotary valve 108 during the heating and/or deprotecting process described herein, wherein low pressure inert gas is directed (bubbled) into a lower portion of reaction vessel 4 to stir the reactants and/or flush evaporated deprotecting base from the headspace of the reaction vessel.

As another example, as discussed herein, in certain embodiments, as also depicted schematically in FIG. 2A (and FIG. 6A), the system can include a rotary valve 140 that can rotate among multiple positions (e.g., two positions) to fluidly connect reaction vessel 4 through opening 12, flow path 22, and flow path 52 with solid support and/or LPPS support (e.g., a soluble tag) containers 50a, 50b, and 50c. Alternately, rotary valve 140 can rotate among multiple positions (e.g., two positions) to fluidly connect reaction vessel 4 through opening 12, flow path 22, and a flow path 152, wherein flow path 152 is in fluid communication with a plurality of flow paths 151a, 151b, and 151c, which in turn fluidly connect flow path 152 with a plurality of product containers 150a, 150b, and 150c, respectively. This can allow the passage of product (e.g., peptide and/or peptide linked to a solid support and/or LPPS support (e.g., a soluble tag)) from reaction vessel 4 into container(s) 150a, 150b, and 150c. Again, the skilled artisan will appreciate that the number of product containers 150a, 150b, and 150c, and corresponding flow paths 151a, 151b, and 151c, can vary and is not limited to the number depicted in FIG. 2A (and FIG. 6A). FIG. 2A (and FIG. 6A) schematically depicts rotary valve 140 in a closed position relative to flow paths 52 and 152 (e.g., in a closed position relative to flow paths 52 and 152 during a deprotecting step as described herein).

As another example, as discussed herein, in certain embodiments, as also depicted schematically in FIG. 2B (and FIG. 6B), the system can include a rotary valve 140 that can rotate among multiple positions (e.g., two positions) to fluidly connect reaction vessel 4 through opening 204, flow path 214, and flow path 52 with solid support and/or LPPS support (e.g., a soluble tag) containers 50a, 50b, and 50c. Alternately, rotary valve 140 can rotate among multiple positions (e.g., two positions) to fluidly connect reaction vessel 4 through opening 204, flow path 214, and flow path 152, wherein again flow path 152 is in fluid communication with a plurality of flow paths 151a, 151b, and 151c, which in turn fluidly connect flow path 152 with a plurality of product containers 150a, 150b, and 150c, respectively. Again, this can allow the passage of product (e.g., peptide and/or peptide linked to a solid support and/or LPPS support (e.g., a soluble tag)) from reaction vessel 4 into container(s) 150a, 150b, and 150c. Also again, the skilled artisan will appreciate that the number of product containers 150a, 150b, and 150c, and corresponding flow paths 151a, 151b, and 151c, can vary and is not limited to the number depicted in FIG. 2B (and FIG. 6B). FIG. 2B (and FIG. 6B) schematically depicts rotary valve 140 in a closed position relative to flow paths 52 and 152 (e.g., in a closed position relative to flow paths 52 and 152 during a deprotecting step as described herein).

Peptide synthesis system 2 can also include one or more flow paths, vents, containers, valves, controllers, and the like, for example, for the removal of waste (e.g., excess reactants, solvents, extraction waste, etc.) from the peptide synthesis system. The waste can be in gas, liquid and/or solid form and the skilled artisan will appreciate appropriate types of flow paths and containers for removing the same from the peptide synthesis system. For example, in certain embodiments, as discussed herein, waste container 130 can be fluidly connected to reaction vessel 4 via flow path 132 and rotary valve 108 when in the appropriate open position to allow the passage of waste products (e.g., extraction waste) from reaction vessel 4 to waste container 130. FIGS. 2A and 2B (and FIGS. 6A and 6B) schematically depict rotary valve 108 in a closed position relative to flow path 132 (e.g., in a closed position relative to flow path 132 during a deprotecting step as described herein). As another example, in certain embodiments, as discussed herein, the open position of valve 120 can allow flow of gas from the upper interior portion (headspace) of reaction vessel 4 through opening 14 or opening 206 and flow path 24 or flow path 216 to a waste recovery zone, such as a vent and/or waste container (not shown) to permit purging/venting of gas from reaction vessel 4 (e.g., during a deprotecting step as described herein). FIGS. 2A and 2B (and FIGS. 6A and 6B) schematically depict valve 120 in an open position relative to flow path 24 or 216 (e.g., in an open position relative to flow path 24 or 216 during a deprotecting step as described herein).

Thus, generally, FIGS. 2A and 2B (and FIGS. 6A and 6B) illustrate an exemplary system for the delivery of solvents, reactants (amino acids, deprotecting bases, activators, extraction solvent, etc.), solid phase resins, LPPS support (e.g., a soluble tag), gases, etc. from their respective sources to reaction vessel 4 and the further delivery of products and by-products (peptides, gas, liquid, and/or solid waste, etc.) from reaction vessel 4 to their respective destinations. It will be understood that the particular flow paths and valve locations illustrate, rather than limit, the present disclosure.

At least partially reiterating from above, the peptide synthesis system typically includes at least one controller operatively associated with, for example, numerous electrical components of the system (e.g., the microwave source, sensors, and solenoid and/or other motor-operated valves). The at least one controller can include one or more computers, computer data storage devices, programmable logic devices (PLDs) and/or application-specific integrated circuits (ASIC). A suitable computer can include one or more of each of a central processing unit or processor, computer hardware integrated circuits or memory, user interface, peripheral or equipment interface for interfacing with other electrical components of the system, and/or any other suitable features. The controller(s) can respectively communicate with electrical components of the system by way of suitable signal communication paths. In FIGS. 2A and 2B (and FIGS. 6A and 6B), representative signal communication paths associated with a controller are schematically depicted and designated by numerals *2 (signal communication paths) and *1 (controller), respectively. Processes of this disclosure can be controlled (e.g., at least partially controlled) in response to the execution of computer-based algorithms operatively associated with the at least one controller *1.

Solid phase peptide synthesis processes, including batch-based processes, are known and thus the present disclosure does not provide detailed information on the same. Reference is made, for example, to the pioneering work R. B. Merrifield (1963) “Solid Phase Peptide Synthesis I, The Synthesis of a Tetrapeptide,” J. Am. Chem. Soc. 85 (14), 2149-2154). Accordingly, a detailed discussion of solid phase peptide synthesis processes is not provided.

Systems suitable for conducting solid phase peptide synthesis, including batch-based processes, are also known. Exemplary systems for conducting solid phase peptide synthesis include, for example, the LIBERTY line of instruments commercially available from CEM Corporation of Matthews N.C.

Liquid phase peptide synthesis processes, including batch-based processes, are also known and thus the present disclosure does not provide detailed information or detailed discussion thereof. Systems suitable for conducting liquid phase peptide synthesis, including batch-based processes, are also known and are commercially available.

Reference is also made to exemplary US patents dealing with the subject of solid phase peptide synthesis (including exemplary systems and/or processes) including without limitation U.S. Pat. Nos. 7,393,920; 7,550,560; 7,563,865; 7,939,628; 7,902,488; 7,582,728; 8,153,761; 8,058,393; 8,426,560; 8,846,862; 9,211,522; 9,669,380; 10,052,607; 10,308,677; 10,125,163; 10,858,390; and 10,239,914. The contents of each of these are incorporated entirely herein by reference.

The deprotecting processes and/or SPPS processes and/or LPPS processes of the present disclosure may be used as part of a SPPS process and/or LPPS process that do not include (that eliminate) washing and/or draining after each coupling step and/or that add deprotection base directly to a coupling solution from a preceding coupling step without any draining after coupling, such as disclosed in, for example, U.S. Pat. Nos. 10,308,677; 10,125,163; 10,858,390; and 10,239,914. Such SPPS processes may be referred to generally as “High Efficiency SPPS (HE-SPPS).”

FIG. 3 is a flow chart schematically depicting the steps of a cycle of a conventional solid phase peptide synthesis (SPPS) process broadly designated at 300. A deprotection step 302 is carried out in a reaction vessel as known in the art by adding a deprotection solution including a deprotecting base to the reaction vessel. The deprotection solution is then drained (step 304) following which a washing liquid (e.g., methanol or isopropanol) is added to the vessel for a washing step 306 carried out repetitively with five repetitions being typical. The washing solution is then removed in a second draining step 308 and thereafter a coupling step 310 takes place in the reaction vessel. A coupling solution is then removed in a third draining step 312 followed by a second washing step 314, again typically repeated five times, followed by a fourth draining step 316.

Thus, FIG. 3 illustrates draining and washing steps following each of a deprotecting step and a coupling step of a conventional SPPS cycle. It will be understood that FIG. 3 is schematic, and that there are many details about one SPPS cycle that could be added, but that FIG. 3 illustrates the concept sufficiently for the skilled person to understand both it and the present invention.

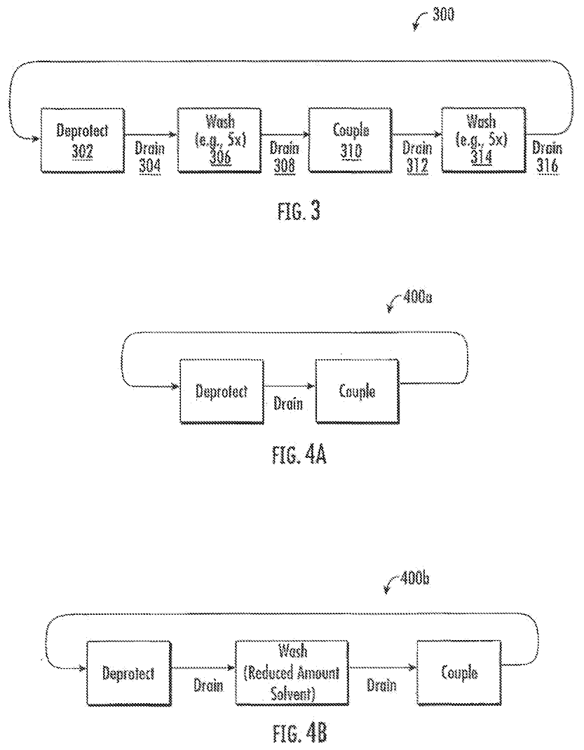

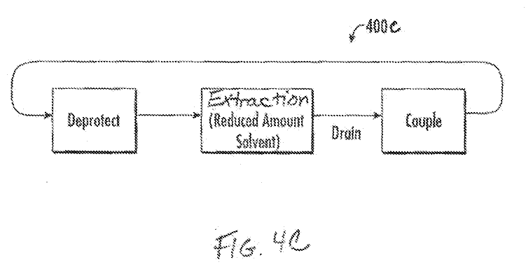

In contrast, FIGS. 4A and 4B are flow charts schematically depicting the steps of a cycle of SPPS processes in accordance with exemplary embodiments of the present disclosure, broadly designated at 400a and 400b, respectively, each as described in more detail herein.

FIG. 4A schematically depicts a deprotecting step/a deprotecting-coupling cycle of a SPPS process in accordance with exemplary embodiments of the present disclosure described in more detail herein, in which a deprotection solution including a deprotecting base is added to the coupling solution from the preceding coupling cycle, without any washing or draining steps between the coupling step of the previous cycle and the addition of the deprotection solution of the deprotecting step of the successive cycle. FIG. 4A schematically depicts optionally draining liquid remaining in the vessel post-deprotection (e.g., solvent, residual deprotecting agent, if any, etc.). FIG. 4A further schematically depicts that washing steps after each deprotecting step (before the next coupling step) in accordance with exemplary embodiments of the present disclosure described herein can be eliminated. FIG. 4A thus schematically depicts an exemplary embodiment of the present disclosure in which washing steps following a deprotecting step (and also washing steps and/or draining steps following a coupling step) of a SPPS cycle may be eliminated.

FIG. 4B schematically depicts a deprotecting step/a deprotecting-coupling cycle of a SPPS process in accordance with other exemplary embodiments of the present disclosure described in more detail herein, in which a deprotection solution including a deprotecting base is added to the coupling solution from the preceding coupling cycle, without any washing or draining steps between the coupling step of the previous cycle and the addition of the deprotection solution of the deprotecting step of the successive cycle. FIG. 4B schematically depicts optionally draining liquid remaining in the vessel post-deprotection (e.g., solvent, residual deprotecting agent, if any, etc.). FIG. 4B further schematically depicts one or more washing steps after each deprotecting step and before the next coupling step, followed by one or more additional optional draining step(s). In contrast to conventional SPPS processes such as schematically depicted in FIG. 3, however, FIG. 4B depicts that any post-deprotecting washing steps may use reduced amounts of solvent in accordance with other exemplary embodiments described herein. FIG. 4B thus schematically depicts an exemplary embodiment of the present disclosure in which the volume of a washing liquid (e.g., solvent) may be reduced, relative to the volume of solvent used post-deprotection in conventional SPPS processes.

FIG. 4C is flow chart schematically depicting a deprotecting step/a deprotecting-coupling cycle of a LPPS process broadly designated at 400c in accordance with other exemplary embodiments of the present disclosure described in more detail herein. A deprotection solution including a deprotecting base is added to a reaction vessel, with or without washing and/or draining steps between a coupling step of a previous deprotecting-coupling cycle and the deprotecting step of the successive cycle (e.g., coupling solution from the preceding coupling cycle may or may not be present in the reaction vessel to which deprotecting solution is added). FIG. 4C also schematically depicts one or more extraction step(s) after deprotection in which an extraction solvent (e.g., an aqueous solvent) is added to the reaction vessel after deprotection and before the next successive coupling, to form a waste layer including residual deprotecting agent remaining in the reaction vessel after deprotection, which waste layer is removed (e.g., drained) from the reaction vessel. FIG. 4C further schematically depicts one or more draining step(s) to remove waste (e.g., an aqueous waste layer including residual deprotecting base). FIG. 4C depicts that post-deprotecting extraction steps may use reduced amounts of extraction solvent in accordance with other exemplary embodiments described herein, relative to the volume of extraction solvent used post-deprotection in conventional LPPS processes. FIG. 4C thus schematically depicts extracting residual deprotecting base from the reaction vessel after removing the protecting group and before a successive coupling step of a deprotection-coupling cycle wherein a total volume of extraction solvent used in all extraction steps associated with the deprotection-coupling cycle (the total combined volume of extraction solvent for all extraction steps of a single deprotection-coupling cycle) is 2 times (2×) or less than the total amount (total volume) of the deprotection reaction mixture (e.g., the deprotection reaction solution) of the deprotection step of that deprotecting-coupling cycle.

The following examples are provided for illustration only and are not to be in any way construed as limiting the present invention. The examples demonstrate that even using small amounts of deprotecting base may result in essentially complete deprotection and scavenging of the protecting group (e.g., Fmoc protecting group) with only residual base left, which may be small enough to minimize issues for the next coupling step.

### Example 1

### Analysis of One-Pot Synthesis of JR 10 Mer Using Low Base Concentrations, with and without Post-Deprotection Washing and with and without Headspace Flushing

JR 10 mer is synthesized using solid phase peptide synthesis using a commercially available automated microwave peptide synthesizer (e.g., from the Liberty line of microwave peptide synthesizers commercially available from CEM Corporation, Matthews, NC, such as Liberty PRIME 2.0) at a 0.1 mmol scale. PEG-PS resin (e.g., Rink Amide ProTide Resin LL commercially available from CEM Corporation) or PS resin (e.g., Fmoc-Rink Amide MBHA PS commercially available from CEM Corporation) is used as the solid phase resin support, and coupling reactions are performed in the presence of Fmoc-protected amino acids (AA).

Deprotection reactions are performed by adding a pyrrolidine/dimethylformamide (DMF) deprotection reagent (the deprotection solution) to an undrained post-coupling mixture (the coupling solution). The concentration of pyrrolidine (e.g., volume percent pyrrolidine in the reaction vessel based on the total volume of a deprotection reaction mixture (e.g., a deprotection reaction solution) including the added pyrrolidine/DMF deprotection solution and the undrained coupling solution from the preceding coupling reaction) is noted in Table 1 below. Microwave power is regulated to provide a deprotection temperature of 110° C. and a deprotection reaction time as also noted in Table 1 below.

Table 1 further indicates whether post-deprotection washing and/or headspace flushing is used. For samples in Table 1 wherein “Headspace Flushing” is indicated as “ON,” a nitrogen gas stream is directed through the headspace of the reaction vessel to purge headspace gas from the reaction vessel in accordance with embodiments of the present disclosure described herein (e.g., directing a pressurized nitrogen gas stream into the reaction vessel through an entry port such as shown in FIGS. 1A and 1B, through the headspace, and out of the reaction vessel through an exit port such as a vent port such as shown in FIGS. 1A and 1B). For examples in Table 1 wherein “Headspace Flushing” is indicated as “OFF,” headspace flushing as described herein is not used. For examples in Table 1 wherein “Post-Deprotection Washing” is used, the washing step includes washing two times using 4 mL of DMF after each deprotection step.

Following completion of synthesis of the JR 10 mer, the JR 10 mer is cleaved from the solid phase and crude purity of the resultant JR 10 mer is analyzed. The results are also reported in Table 1 below.

TABLE 1

| | Deprotection | Deprotection | Post- | | |
|---|---|---|---|---|---|
| | (% | Time | Deprotection | Headspace | Crude |
| Entry | pyrrolidine) | (sec) | Washing | Flushing | Purity |
| 1 | 4.5 | 40 | 2 × 4 mL DMF | OFF | 77% |
| 2 | 4.5 | 40 | 2 × 4 mL DMF | ON | 79% |
| 3 | 4.5 | 40 | None | OFF | 16% |
| 4 | 4.5 | 40 | None | ON | 69% |
| 5 | 4.5 | 80 | None | OFF | 20% |
| 6 | 4.5 | 80 | None | ON | 68% |
| 7 | 3.5 | 80 | None | OFF | 34% |
| 8 | 3.5 | 80 | None | ON | 86% |
| 9 | 3 | 80 | None | OFF | 56% |
| 10 | 3 | 80 | None | ON | 84% |
| 11 | 3 | 80 | None | ON | 73% |
| 12 | 3 | 40 | None | ON | 73% |
| 13 | 2 | 80 | None | OFF | 36% |
| 14 | 2 | 80 | None | ON | 65% |
| 15 | 3 | 80 | 2 × 4 mL DMF | ON | 81% |
| 16 | 4.5 | 40 | 2 × 4 mL DMF | ON | 74% |
| 17 | 3 | 80 | None | ON | 65% |

Notes:
1. Fmoc-Rink Amide ProTide ™ LL resin (0.20 meq/g substitution) is used for all experiments except Entry 11 that uses Fmoc-Rink Amide MBHA PS resin (0.33 meq/g substitution).
2. N-butylpyrrolidinone (NBP) is used in place of DMF for Entries 16 and 17.

The results for the JR peptide synthesis show that a high purity result can be obtained without any washing when using the headspace flushing as described herein with each deprotection step. For example, without being bound by any explanation or theory and without limiting the scope of the invention, it is currently believed that directing an inert gas (nitrogen gas) through the reaction vessel (e.g., into the reaction vessel through an entry port, through the headspace, and out of the reaction vessel through an exit port (a vent port) such as shown in FIGS. 1A and 1B) may result in both a higher gas exchange rate above the deprotection solution and a top down directional flow which pushes condensation back into the reaction vessel where it is then reheated

In addition, also without being bound by any explanation or theory and without limiting the scope of the invention, it is currently believed that Example 1 demonstrates that pyrrolidine base, which has a lower boiling point (87° C.) compared to piperidine (106° C.), may be significantly evaporated in an Fmoc removal step and that even using small amounts thereof (as low as 2 vol %) may result in essentially complete deprotection and scavenging of the Fmoc group with only residual base left, which may be small enough to minimize issues for the next coupling step. It is also currently believed that the processes can allow high purity synthesis of JR with a complete cycle waste of only 4.25 mL per amino acid and a total cycle time of approximately 3.5 minutes at the common 0.1 mmol research scale.

In recent years, greener solvent replacements have been explored for SPPS. There above experiments also assessed the processes of the present disclosure using a green solvent alternative. To evaluate this, JR sequence is synthesized with N-butylpyrrolidinone (NBP) completely replacing DMF under both control conditions using post-deprotection washing and with the wash-free conditions (Table 1, entries 16 and 17). While NBP shows a reduction in purity compared to DMF, it is still successful in producing the target in relatively high purity with both the wash-based and wash-free conditions. This result indicates that the processes disclosed herein may also work with solvent alternatives to DMF.

### Example 2

### Analysis of One-Pot Synthesis of ⁶⁵⁻⁷⁴ACP, Liraglutide, and ¹⁻⁴²-Amyloid Sequences Using Protecting Composition Having a Low Base Concentration without Post-Deprotection

### Washing and with Headspace Flushing

Other well-known difficult sequences, namely, the ⁶⁵⁻⁷⁴ACP (acyl carrier protein), ¹⁻⁴²β-amyloid and liraglutide (Entries 1, 2, and 3 in Table 2 below), are next investigated. The sequences are synthesized using solid phase peptide synthesis with a commercially available automated microwave peptide synthesizer (e.g., from the Liberty line of microwave peptide synthesizers commercially available from CEM Corporation, Matthews, NC, such as Liberty PRIME 2.0) on a 0.1 mmol scale. ⁶⁵⁻⁷⁴ACP and Liraglutide are synthesized on Fmoc-Gly-Wang-ProTide resin (0.24 meq/g substitution) and ¹⁻⁴²β-Amyloid is synthesized on Fmoc-Ala-Wang-ProTide resin (0.23 meq/g substitution). Coupling reactions are performed in the presence of Fmoc-protected amino acids (AA).

Liraglutide synthesis is further investigated in a simulated run at 0.1 mmol scale in a 35 mL reaction vessel using deprotection and coupling conditions for a large-scale (25 mmol) production method previously developed in a 15 liter reaction vessel (Entry 4 in Table 2 below). This method employs a lower temperature deprotection and coupling method that is limited to 80° C. To test the robustness of this process for production scales the deprotection step is extended to 8 min at 80° C. As reported in Table 2, this method yields Liraglutide with crude purity that matches previous results obtained with wash methods.

Deprotection reactions are performed by adding a pyrrolidine/dimethylformamide (DMF) deprotection reagent (the deprotection solution) to an undrained post-coupling mixture (the coupling solution). The concentration of pyrrolidine (e.g., volume percent pyrrolidine in the reaction vessel based on the total volume of a deprotection reaction mixture (e.g., a deprotection reaction solution) including the added pyrrolidine/DMF deprotection solution and the undrained coupling solution from the preceding coupling reaction) is 3 vol %. A 3 vol % pyrrolidine concentration is chosen as a middle value utilization of process toward the synthesis of these sequences. Microwave power is regulated to provide a deprotection temperature and a deprotection reaction time as also noted in Table 2 below.

Following completion of synthesis of the sequences, the sequences are cleaved from the solid phase and crude purity of the resultant sequences is analyzed. The results are reported in Table 2 below. Specifically, the column of Table 2 labeled “Crude Purity (Wash-free)” reports crude purity of peptides produced using a deprotection process in accordance with embodiments of the present disclosure described herein (including head space flushing and no post-deprotection washing). For comparison, the column of Table 2 labeled “Crude Purity (Wash based)” reports crude purity of peptides produced using a wash-based deprotection process (without head space flushing and with post-deprotection washing).

TABLE 2

| | | | | | | Crude | Crude |
|---|---|---|---|---|---|---|---|
| | | Depro- | | Depro- | | Purity | Purity |
| | | tection | | tection | | (Wash- | (Wash |
| Entry | Peptide | Temp. | | Time | | free) | based) |
| 1 | ⁶⁵⁻⁷⁴ACP | 110° | C. | 80 | sec | 91% | 91% |
| 2 | ¹⁻⁴²β-amyloid | 110° | C. | 80 | sec | 67% | 69% |
| 3 | Liraglutide | 110° | C. | 80 | sec | 73% | 68% |
| 4 | Liraglutide | 80° | C. | 8 | min | 75% | — |

The results for the ⁶⁵⁻⁷⁴ACP, ¹⁻⁴²β-amyloid and Liraglutide sequences also show that a high purity result can be obtained without any washing when using the headspace flushing as described herein in each deprotection step.

The foregoing Examples 1 and 2 demonstrate that embodiments of the present disclosure including a deprotection step described herein can provide an improved process that can eliminate one or more (e.g., all) washing steps for solid phase peptide synthesis (e.g., can eliminate post-deprotection washing steps). In some embodiments, the process may use low amounts of deprotecting base (e.g., about 3-4 vol % pyrrolidine) for Fmoc removal; and/or heating (e.g., microwave heating at 80-110° C.); and/or base removal from the deprotection solution with elevated temperatures and/or nitrogen purging (headspace flushing), which may result in a low enough remaining base so as to eliminate the need for washing before the next amino acid is added. Examples 1 and 2 demonstrate significant robustness even on longer, more difficult sequences such as Liraglutide. Thus, the processes may provide a major savings in solvent and time.

### Example 3

### Wash-Free Production Scale Synthesis

In preparation for testing the wash-free method at a production scale, 25 mmol scale deprotection and coupling conditions are investigated by synthesizing liraglutide at research scale (0.1 mmol) in a 35 mL reaction vessel, as discussed in Example 2 above; this method employs lower temperature conditions for deprotection (8 min at 80° C.) and coupling (5 min at 80° C.). Also as reported in Example 2 above, this method yields liraglutide with crude purity that matches previous results obtained with wash-based methods at research and production scales.

Having established the high enantiomeric and chromatographic purities using large-scale reaction conditions for 0.1 mmol scale synthesis, the 25 mmol wash-free synthesis of liraglutide is conducted on the Liberty PRO large-scale microwave peptide synthesizer. Based on results from an initial optimization experiment involving 6 amino acid couplings, exemplary conditions for a 25 mmol wash-free synthesis may include without limitation (i) 2.5% pyrrolidine concentration in the reaction vessel, (ii) 10 min at 90° C. deprotection and 5 min at 80° C. coupling, and (iii) 85 psi nitrogen pressure of directed headspace flushing during each deprotection. 4 equivalent excess of regular amino acids is used and only 2 equivalents of Fmoc-Lys(palmitoyl-Glu-OtBu)-OH for coupling. Under these conditions, the 25 mmol wash-free synthesis of liraglutide generates a total waste of 28.4 L as compared to 139.7 L from the wash-based 25 mmol run which implies an overall waste reduction of approximately 80%. Liraglutide samples from wash-based and wash-free 25 mmol syntheses show very similar (77-78%) crude purities. These results confirm the general applicability of wash-free methodology for research scale as well as production scale synthesis of peptides. Future use of this technique in large-scale peptide drug production would be helpful in reducing enormous amounts of waste generated from SPPS.

The potential for epimerization with this new wash-free method is evaluated by measuring the occurrence of D-amino acids in the liraglutide samples. Liraglutide samples synthesized by both the research and production scale wash-free methods are analyzed using an established method (C.A.T. GmbH). See Gerhardt, J.; Nicholson, G. J., Validation of a GC-MS Method for Determination of the Optical Purity of Peptides. GmbH, C. A. T., Ed. The results from the crude and corresponding purified samples are then compared to a commercial sample of liraglutide (Victoza®) as shown in Table 3. The results show very low levels of epimerization with well over 99.5% control of stereochemistry for each amino acid in the liraglutide sequence using the production method. This demonstrates that all epimerization related impurities are below the critical 0.5% limit for any new specified peptide-related impurity. Meeting this limit is required for an application of a synthetic peptide as a substitute for an approved peptide drug of recombinant deoxyribonucleic acid (rDNA) origin with an abbreviated new drug application (ANDA). The results demonstrate that the processes of the present disclosure may be used not only in research and development but also production processes that require stringent purity standards.

TABLE 3
Epimerization data for liraglutide samples

| | D-Enantiomer | | | | | |
|---|---|---|---|---|---|---|
| | Table 2, Entry 3 | | Table 2, Entry 4 | | | |
| | (Crude) | Purified | (Crude) | Purified | Purified | |
| | 0.1 mmol | 0.1 mmol | 0.1 mmol | 0.1 mmol | 25 mmol | VICTOZA ® |
| Residue | Research Method | Research Method | Production Method | Production Method | Production Run | Lot #FS61B71 |
| Alanine | 0.15% | 0.10% | 0.12% | 0.10% | 0.10% | <0.10% |
| Valine | <0.10% | <0.10% | <0.10% | <0.10% | <0.10% | <0.10% |
| Threonine | <0.10% | <0.10% | <0.10% | <0.10% | <0.10% | <0.10% |
| | <0.10% | <0.10% | <0.10% | <0.10% | <0.10% | <0.10% |
| | D-allo | D-allo | D-allo | D-allo | D-allo | D-allo |
| | <0.10% | <0.10% | <0.10% | <0.10% | <0.10% | <0.10% |
| | L-allo | L-allo | L-allo | L-allo | L-allo | L-allo |
| Isoleucine | <0.10% | <0.10% | <0.10% | <0.10% | <0.10% | <0.10% |
| | 0.11% | <0.10% | <0.10% | <0.12% | <0.12% | <0.10% |
| | D-allo | D-allo | D-allo | D-allo | D-allo | D-allo |
| | <0.10% | <0.10% | <0.10% | <0.13% | <0.14% | <0.11% |
| | L-allo | L-allo | L-allo | L-allo | L-allo | L-allo |
| Leucine | 0.12% | 0.16% | 0.14% | 0.15% | 0.13% | 0.13% |
| Serine | 0.31% | 0.10% | 0.21% | <0.10% | <0.10% | 0.38% |
| Aspartic Acid | 0.36% | 0.38% | 0.17% | 0.33% | 0.18% | 0.12% |
| Phenylalanine | 0.19% | 0.22% | 0.15% | 0.22% | 0.14% | 0.13% |
| Glutamic Acid | 0.23% | 0.37% | 0.23% | 0.30% | 0.25% | 0.16% |
| Tyrosine | 0.15% | 0.10% | <0.10% | 0.14% | <0.10% | 0.18% |
| Lysine | 0.13% | <0.10% | 0.15% | 0.11% | 0.13% | 0.11% |
| Arginine | 0.23% | 0.11% | 0.24% | 0.16% | 0.10% | 0.13% |
| Tryptophan | 0.23% | <0.10% | 0.22% | 0.16% | not | 0.25% |
| | | | | | determined | |
| Histidine | 0.72% | 0.72% | 0.40% | 0.34% | 0.47% | 0.57% |

### Example 4

### Protein Synthesis

The capabilities of this process are then tested further by the synthesis of two proteins with sequence lengths >80 amino acids, proinsulin and barstar. Linear synthesis of long sequences by SPPS is challenging due to the iterative accumulation of impurities and increased susceptibility for aggregation to occur. The proinsulin 86-mer and barstar 89-mer sequences are chosen for synthesis as they were previously synthesized using a fast flow methodology at 1% and 2% overall yield, respectively. The fast flow approach advantageously provides a very fast synthesis time of only ˜2.5 minutes per amino acid cycle at small synthesis scales (0.035 mmol for proinsulin; 0.027 mmol for barstar). However, the process requires a large excess of amino acid (˜100 equivalents) and wash solvent (˜90 mL per amino acid).

To account for the potential increased synthesis difficulty of these longer sequences, a higher coupling concentration is used with 10 equivalents of amino acids and the deprotection time (2 minutes) and coupling time (4.5 minutes) are extended. The pyrrolidine concentration is also increased (3.8% for wash-free method and 6.8% with a more conservative 3×4 mL wash method) to quench the larger excess of activated amino acid. These conditions result in a cycle time of 7.3 minutes per amino acid, and a total waste of 5.5 mL per amino acid at 0.1 mmol synthesis scale. Using this wash-free method, both proteins are obtained with similar crude purity as when washing is utilized. The crude proinsulin and barstar samples are then purified by reversed-phase HPLC which results in 2.4% and 3.4% overall yield, respectively. Purified samples of proteins are identified by deconvoluted mass spectra showing 9395 Da and 10210 Da for proinsulin and barstar, respectively. These protein synthesis examples demonstrate that the processes of the present disclosure can be robust for generating high purity results even for long and challenging sequences.

The foregoing demonstrates wash-free processes for solid phase synthesis of peptides and proteins. The processes of the present disclosure may not negatively impact peptide purity versus controls with washing and can provide high purity and rapid synthesis times, for example when combined with elevated temperature reaction conditions. Compared to traditional SPPS, the processes according to the present disclosure can provide up to a 95% reduction in waste generated.

The foregoing also demonstrates successful application of the processes of the present disclosure at 25 mmol scale on a large-scale microwave peptide synthesizer and that the processes of the present disclosure may be readily scalable. This may allow for larger reaction vessel sizes (e.g., up to 15 liters) at which the processes of the present disclosure may be applied for synthesis scales above 200 mmol per batch. At these larger scales, many hundreds of liters of solvent can be saved per batch of peptide synthesized. Additionally, by largely reducing solvent as a material cost, the processes of the present disclosure may provide a significant boost toward overcoming cost barriers associated with the use of more expensive and potentially less efficient green solvents for SPPS.

### Methods

### Synthesis

(a) Peptides: All peptides are synthesized using automated microwave synthesis conditions on a CEM Liberty PRIME 2.0 system at 0.1 mmol scale using the one-pot coupling/deprotection methodology. See, e.g., U.S. Pat. No. 10,239,914; Singh, S. K.; Collins, J. M., New Developments in Microwave-Assisted Solid Phase Peptide Synthesis. In Peptide Synthesis: Methods and Protocols, Hussein, W. M.; Skwarczynski, M.; Toth, I., Eds. Springer US: New York, NY, 2020; 95-109. Method details involving reaction time, temperature, concentration of deprotection reagent etc. are described in Tables 1 and 2. Couplings are performed for 30 seconds at room temperature followed by 60 seconds at 105° C. using Fmoc-amino acid (1.0 mL, 0.5 M in DMF, 5 equivalents), DIC (1.0 mL, 0.75 M in DMF, 7.5 equivalents) and Oxyma (1.5 mL, 0.26 M in DMF, 4 equivalents). Fmoc deprotection step(s) using 3 vol % pyrrolidine (e.g., see Example 2) is initiated by adding 0.75 mL of pyrrolidine/DMF (17% v/v) directly to the undrained post-coupling solution (optimization experiments are performed by adding 0.75 mL of 11.3-25% v/v pyrrolidine/DMF as described in Table 1). Headspace flushing pressure of 15 psi is used during the deprotection step. The wash-based method uses 2×4 mL DMF post-deprotection washings. The cycle involving deprotection-coupling (for wash-free) or deprotection-washing-coupling (for wash-based) runs is automatically performed for all amino acid residues in the peptide sequence. JR-10 mer is synthesized on Fmoc-Rink Amide ProTide™ LL resin (0.20 meq/g substitution) or Fmoc-Rink Amide MBHA PS resin (0.33 meq/g substitution). ⁶⁵⁻⁷⁴ACP and Liraglutide are synthesized on Fmoc-Gly-Wang-ProTide resin (0.24 meq/g substitution) and ¹⁻⁴²β-Amyloid is synthesized on Fmoc-Ala-Wang-Portside resin (0.23 meq/g substitution).

(b) Wash-Free Production Scale Liraglutide Synthesis: Liraglutide is synthesized at 25 mmol scale using Fmoc-Gly-Wang-ProTide resin (0.24 meq/g substitution) in a 3 L reaction vessel on the Liberty PRO microwave peptide synthesizer. Couplings are performed for 5 min at 80° C. using Fmoc-amino acid (200 mL, 0.5 M in DMF), DIC (50 mL, 4 M in DMF) and Oxyma (225 mL, 0.33 M in DMF). After draining the post-coupling mixture, Fmoc deprotection step is performed for 10 min at 90° C. by adding 50 mL of pyrrolidine/DMF (15% v/v) followed by additional DMF (250 mL) to obtain a final concentration of 2.5% pyrrolidine in the reaction vessel. Nitrogen pressure at 85 psi is directed through a spray head in the top of the reaction vessel to facilitate directed flushing of the headspace gas during each deprotection step. Fmoc-Lys(palmitoyl-Glu-OtBu)-OH is coupled using 2 equivalent excess with a wash-based coupling cycle, while all other amino acid residues in the sequence use no washings after the deprotection and coupling steps. Fmoc-His(Boc)-OH is coupled by using 2×30 min at 40° C. method. Wash-based cycles at 25 mmol scale use 4×650 mL DMF and 1×800 mL DMF for post-deprotection washings. The cycles involving deprotection-coupling (for wash-free) or deprotection-washing-coupling (for wash-based) runs are automatically performed for all amino acid residues in the peptide sequence.

(c) Proteins: Proteins are synthesized using automated microwave synthesis conditions on a CEM Liberty PRIME 2.0 system at 0.10 mmol scale using the one-pot coupling/deprotection methodology. See, e.g., U.S. Pat. No. 10,239,914; Singh, S. K.; Collins, J. M., New Developments in Microwave-Assisted Solid Phase Peptide Synthesis. In Peptide Synthesis: Methods and Protocols, Hussein, W. M.; Skwarczynski, M.; Toth, I., Eds. Springer US: New York, NY, 2020; 95-109. Couplings are performed with Fmoc-amino acid (2.0 mL, 0.5 M in DMF), DIC (1.0 mL, 2.0 M in DMF) and Oxyma (1.75 mL, 0.50 M in DMF) for 30 seconds at room temperature followed by 4 minutes at 90° C. Fmoc deprotection step is performed for 2 minutes at 110° C. and initiated by adding 0.75 mL of pyrrolidine/DMF (28% v/v) directly to the undrained post-coupling solution. Headspace flushing pressure of 15 psi is used during the deprotection step. The wash-based method uses 3×4 mL DMF post-deprotection washings. The cycles involving deprotection-coupling (for wash-free) or deprotection-washing-coupling (for wash-based) runs are automatically performed for all amino acid residues in the peptide sequence. Proinsulin 86-mer and Barstar 89-mer proteins are synthesized on Fmoc-Rink Amide ProTide™ LL resin (0.18 meq/g substitution).

### Resin Cleavage

(a) Peptides: The peptidyl resin is washed with DCM (3×5 mL) after synthesis. Cleavage is performed for 30 min at 38° C. using 5 mL of a freshly prepared cleavage cocktail [TFA/TIS/H₂0/DODT (92.5/2.5/2.5/2.5)]. The TFA solution is collected by filtration and ice cold ethyl ether is added followed by centrifugation at 3500 rpm for 5 minutes to obtain the crude peptide as white pellet.

(b) Proteins: The peptidyl resin is washed with DCM (3×15 mL) after synthesis. Cleavage is performed for 5 hours at room temperature using a slow cleavage method by adding 7.5 mL of [TFA/TIS/H₂O/DODT (6/0.5/0.5/0.5)] followed by gradual addition of 4 mL TFA every hour for 3 hours. After the third addition (final conc. TFA/TIS/H₂O/DODT (18/0.5/0.5/0.5) the cleavage is allowed to react for an additional 2 hours. The TFA solution is collected by filtration and ice cold ethyl ether is added followed by centrifugation at 3900 rpm for 3 minutes to obtain the crude peptide as a white pellet.

### Analysis

All peptides are lyophilized overnight after dissolving the pellet in 10% acetic acid/deionized water. A lyophilized aliquot of the peptide is taken in deionized water (˜2 mg/mL peptide concentration) and a clear solution is obtained by addition of acetonitrile, ammonium hydroxide (up to 1%), or acetic acid (up to 9%) followed by sonication. Protein samples (barstar and proinsulin) are dissolved by sonicating in a solution of H₂O/ACN/AcOH (8:1:1) for 1 hour. The peptide/protein solution are analyzed on a Vanquish UHPLC system (Thermo Fisher; Waltham, MA, USA) with a Waters ACQUITY UPLC BEH C8 reversed-phase column (100×2.1 mm i.d., 1.7 μm, 130 Å; Waters Corporation, Milford, MA, USA) coupled to an Exactive™ Plus Orbitrap™ mass spectrometer (Thermo Fisher; Waltham, MA, USA) via an ESI source (operated in positive polarity mode). Deconvoluted mass spectra for proteins are obtained using UniDec (Universal Deconvolution) Version 6.0.1 developed by Marty et al. Marty, M. T.; Baldwin, A. J.; Marklund, E. G.; Hoshberg, G. K. A.; Benesch, J. L. P.; Robinson, C. V., Bayesian Deconvolution of Mass and Ion Mobility Spectra: From Binary Interactions to Polydisperse Ensembles. Anal. Chem. 2015, 87 (8), 4370-4376. Analytical runs are performed at a flow rate of 0.5 mL/min with gradient elution of 10-70% B using 0.05% trifluoroacetic acid in water (A) and 0.05% trifluoroacetic acid in acetonitrile (B). The column and autosampler are maintained at 40 and 24° C., respectively for all peptides except ¹⁻⁴²β-amyloid. ¹⁻⁴²β-amyloid is analyzed on a Waters ACQUITY UPLC BEH C8 reversed-phase column (100×2.1 mm i.d., 1.7 μm, 130 Å; Waters Corporation, Milford, MA, USA) with a flow rate of 0.6 mL/min at 70° C. column temperature on a Waters Acquity RP-UPLC system with PDA detector coupled to a 3100 Single Quad mass spectrometer.

### Purification

Lyophilized protein samples (barstar and proinsulin) are dissolved (barstar: 6.4 mg/mL in water with 0.2% ammonium hydroxide and 10 mM DTT; proinsulin: 8 mg/mL in 6 M GdnHCl with 0.1% ammonium hydroxide and 100 mM DTT through sonication for 1 hour at 40° C. Lyophilized liraglutide is dissolved (8.1 mg/mL) in 20% acetonitrile. Samples are filtered with a 0.45 μm regenerated cellulose syringe filter (Phenomenex; Torrance, CA, USA) prior to purification.

Purifications are completed on a CEM Prodigy HPLC System, which includes an integrated heating system (column oven and mobile phase heater) to enable high-efficiency elevated temperature operation. Barstar and proinsulin purifications are performed at 60° C. using a Waters Protein XBridge C4 column (19×150 mm, 5 μm, 300 Å; Waters Corporation, Milford, MA, USA) with mobile phases consisting of 0.1% trifluoroacetic acid in water (A) and 0.1% trifluoroacetic acid in acetonitrile (B). Liraglutide purifications are performed using the same conditions, but with a Waters XBridge C8 column (19×150 mm, 5 μm, 130 Å).

Optimized gradient conditions are determined by first injecting −10 mg of crude sample on a 10-70% B screening gradient (20 min gradient; ˜3% B/CV) with a flow rate of 27 mL/min. The target peak retention time is then used to calculate (using CEM Focused Gradient Calculator software, version 1.1.673.1159) optimized focused gradients for each purification. The protein samples are purified using focused gradients over 25 min (proinsulin: 27-39% B; barstar: 39-51% B), while liraglutide is purified using a focused gradient over 18 min. (46-55% B).

In the foregoing, examples of embodiments have been disclosed. The present invention is not limited to such exemplary embodiments. In the foregoing, descriptions of sequences of steps or other actions are described for purposes of providing examples, and not for the purpose of limiting the scope of this disclosure (e.g., where appropriate, steps or actions may be performed in different sequences than described above, and steps and actions may be omitted and/or added). The figures are schematic representations and so are not necessarily drawn to scale. Unless otherwise noted, specific terms have been used in a generic and descriptive sense and not for purposes of limitation.

Numerical values provided throughout this disclosure can be approximate, and for each range specified in this disclosure, all values within the range (including end points) and all subranges within the range are also disclosed. Those of ordinary skill in the art will also readily understand that, in different implementations of the features of this disclosure, reasonably different engineering tolerances, precision, and/or accuracy (for example with respect to numerical value(s)) may be applicable and suitable for obtaining the desired result. Those of ordinary skill will accordingly readily understand the meaning, usage, etc. herein of terms such as “substantially,” “about,” “approximately,” and the like. As non-limiting examples, the term “about” can indicate that a numeric value can vary by plus or minus 25%, for example plus or minus 20%, for example plus or minus 15%, for example plus or minus 10%, for example plus or minus 5%, for example plus or minus 4%, for example plus or minus 3%, for example plus or minus 2%, for example plus or minus 1%, for example plus or minus less than 1%, for example plus or minus 0.5%, for example less than plus or minus 0.5%, including all values and subranges therebetween for each of the above ranges.

As used herein, the phrase “and/or” includes any and all combinations of one or more of the associated listed items (e.g., can refer to elements that are conjunctively present in some embodiments and elements that are disjunctively present in other embodiments), and in some embodiments optionally in combination with other elements not specifically identified by the “and/or” phrase. As non-limiting examples, “A and/or B” can refer in some embodiments to A without B; in some embodiments to B without A; in some embodiments to both A and B; etc.

As used herein, the phrase “at least one” in reference to a list of one or more elements can refer to at least one element selected from any one or more of the elements in the list of elements, but not necessarily including at least one of each and every element specifically listed within the list of elements and not excluding any combinations of elements in the list of elements. In some embodiments, elements may be optionally present other than the elements specifically identified within the list of elements to which the phrase “at least one” refers, whether related or unrelated to those elements specifically identified. As non-limiting examples, “at least one of A and B”; “at least one of A or B”; and/or “at least one of A and/or B” can refer in some embodiments to at least one, optionally including more than one, A, with no B present (and optionally including elements other than B); in some embodiments to at least one, optionally including more than more one, B, with no A present (and optionally including elements other than A); in some embodiments to at least one, optionally including more than one, A, and at least one, optionally including more than one, B (and optionally including other elements); etc.

As used herein, indefinite articles “a” and “an” refer to at least one (“a” and “an” can refer to singular and/or plural element(s)).

1. A process for deprotecting a protected amino acid during solid phase peptide synthesis (SPPS) including deprotecting steps and coupling steps, the deprotecting process comprising:
removing a protecting group of a protected amino acid in a reaction vessel with a deprotecting base at a temperature from about 60° C. to about 120° C. to provide a deprotected amino acid, wherein:
the deprotecting base is present in the reaction vessel in an amount greater than zero to about 5 vol %, based on the total volume (100 vol %) of a deprotection reaction mixture in the reaction vessel, and
at least a portion of the deprotecting base evaporates into an upper interior portion of the reaction vessel during the removing of the protecting group of the protected amino acid; and
directing an inert gas through the reaction vessel to remove evaporated deprotecting base from the interior of the reaction vessel during the removing the protecting group of the protected amino acid.

2. The process according to claim 1, wherein the process comprises a coupling step preceding the removing of the protecting group of the protected amino acid, the reaction vessel includes a post-coupling solution after completion of the preceding coupling step, the post-coupling solution is not drained from the reaction vessel after the preceding coupling step and before the removing of the protecting group of the protected amino acid, and the deprotection reaction mixture includes a mixture of a deprotection solution including the deprotecting base and the post-coupling solution from the preceding coupling step.

3. The process according to claim 1, wherein the deprotecting base is present in the reaction vessel in an amount from about 1 vol % to about 5 vol %, based on the total volume (100 vol %) of the deprotection reaction mixture in the reaction vessel.

4. The process according to claim 1, wherein the deprotecting base is present in the reaction vessel in an amount from about 2 vol % to about 4.5 vol %, based on the total volume (100 vol %) of the deprotection reaction mixture in the reaction vessel.

5. The process according to claim 1, wherein the deprotecting base is present in the reaction vessel in an amount from about 3 vol % to about 4.5 vol %, based on the total volume (100 vol %) of the deprotection reaction mixture in the reaction vessel.

6. The process according to claim 1, wherein the directing comprises directing inert gas into an upper interior portion of the reaction vessel through a first opening located in an upper portion of the reaction vessel; and venting inert gas and evaporated deprotecting base from the upper interior portion of the reaction vessel through a second opening located in the upper portion of the reaction vessel.

7. The process according to claim 1, wherein the directing comprises directing inert gas into a lower interior portion of the reaction vessel through a first opening located in a lower portion of the reaction vessel; and venting inert gas and evaporated deprotecting base from the upper interior portion of the reaction vessel through a second opening located in the upper portion of the reaction vessel.

8. The process according to claim 1, wherein the directing comprises directing inert gas into an upper interior portion of the reaction vessel through a first opening located in an upper portion of the reaction vessel, directing inert gas into a lower interior portion of the reaction vessel through a second opening located in a lower portion of the reaction vessel, and venting inert gas and evaporated deprotecting base from the upper interior portion of the reaction vessel through a third opening located in the upper portion of the reaction vessel.

9. The process according to claim 1, wherein the inert gas is nitrogen gas and wherein the process comprises continuously directing the nitrogen gas through the reaction vessel.

10. The process according to claim 1, comprising heating the protected amino acid and the deprotecting base at a temperature from about 90° C. to about 120° C. using microwave radiation.

11. The process according to claim 1, wherein the deprotecting base has a boiling point less than 107° C. and a difference between a temperature of heating the protected amino acid and the deprotecting base during the removing of the protecting group from the protected amino acid and the boiling point of the deprotecting base ranges from about 1° C. to about 50° C.

12. The process according to claim 1, wherein the deprotecting base is pyrrolidine.

13. The process according to claim 2, comprising adding deprotection solution including the deprotecting base to the reaction vessel in an amount sufficient to provide greater than zero to about 5 vol % of the deprotecting base in the reaction vessel, based on the total volume (100 vol %) of a deprotection reaction mixture in the reaction vessel,
wherein the vol % of the deprotecting base of the deprotection solution added to the reaction vessel is greater than the vol % of the deprotecting base of the deprotecting reaction mixture after the deprotection solution is added to the reaction vessel.

14. The process according to claim 13, wherein the vol % of the deprotecting base of the deprotection solution added to the reaction vessel is not greater than about 30 vol %, based on the total volume of the deprotection solution added to the reaction vessel.

15. The process according to claim 1, wherein the protected amino acid is attached to a solid resin support having a resin substitution from 0.10 mmol/g to 0.35 mmol/g.

16. The process according to claim 15, wherein the protected amino acid is attached to a solid resin support having a resin substitution from 0.20 mmol/g to 0.33 mmol/g.

17. The process according to claim 1, wherein:
the deprotecting base is pyrrolidine;
the protected amino acid is attached to a solid PEG-PS (polyethylene glycol-polystyrene) resin support having a resin substitution from 0.20 mm/g to 0.25 mmol/g;
the protecting group of the protected amino acid is a 9-fluorenylmethyloxycarbonyl (Fmoc) protecting group; and
the heating is conducted at a temperature of about 60° C. or higher.

18. A process for deprotecting a protected amino acid during solid phase peptide synthesis (SPPS) including deprotecting steps and coupling steps, the deprotecting process comprising:
removing a protecting group of a protected amino acid attached to a solid resin with a resin substitution from 0.10 mmol/g to 0.35 mmol/g in a reaction vessel with a deprotecting base at a temperature that is no less than 35° C. below the boiling point of the deprotecting base and for a time of 1.5 hours or less to provide a deprotected amino acid, wherein:
the deprotecting base is present in the reaction vessel in an amount greater than zero to about 5 vol %, based on the total volume (100 vol %) of a deprotection reaction mixture in the reaction vessel, and
a majority of the deprotecting base evaporates into an upper interior portion of the reaction vessel during the removing of the protecting group of the protected amino acid; and
directing an inert gas through the reaction vessel to assist in the removal of evaporated deprotecting base from the interior of the reaction vessel during the removing the protecting group of the protected amino acid; and
washing the interior of the reaction vessel after removing the protecting group and before a successive coupling step of one or more deprotection-coupling cycles of the solid phase peptide synthesis using a solvent in an amount that is 2 times or less of a bed volume of resin present in the reaction vessel.

19. A process for solid phase peptide synthesis, comprising:
deprotecting a first protected amino acid at a temperature from about 60° C. to about 120° C. to provide a deprotected amino acid; and
coupling a second amino acid to the deprotected amino acid to form a peptide from the first and second amino acids,
wherein the deprotecting comprises:
removing a protecting group of the protected amino acid in a reaction vessel with a deprotecting base, wherein the deprotecting base is present in the reaction vessel in an amount great than zero to about 5 vol %, based on the total volume (100 vol %) of a deprotection reaction mixture in the reaction vessel, and wherein at least a portion of the deprotecting base evaporates into an upper interior portion of the reaction vessel during the removing of the protecting group; and
directing an inert gas through the reaction vessel to remove evaporated deprotecting base from the interior of the reaction vessel during the removing of the protecting group.

20. The process according to claim 19, wherein the process comprises repeating the deprotecting and coupling to form a peptide comprising the first, second, and one or more successive amino acids.

21. The process according to claim 19, wherein the process comprises a coupling step preceding the deprotecting step, the reaction vessel includes a post-coupling solution after completion of the preceding coupling step, the post-coupling solution is not drained from the reaction vessel after the preceding coupling step and before the deprotecting step, and the deprotection reaction mixture includes a mixture of a deprotection solution including the deprotecting base and the post-coupling solution from the preceding coupling step.

22. The process according to claim 19, wherein the deprotecting base is present in the reaction vessel in an amount from about 1 vol % to about 5 vol %, based on the total volume (100 vol %) of the deprotection reaction mixture in the reaction vessel.

23. The process according to claim 19, wherein the deprotecting base is present in the reaction vessel in an amount from about 3 vol % to about 4.5 vol %, based on the total volume (100 vol %) of the deprotection reaction mixture in the reaction vessel.

24. The process according to claim 19, wherein the directing comprises directing inert gas into an upper interior portion of the reaction vessel through a first opening located in an upper portion of the reaction vessel; and venting inert gas and evaporated deprotecting base from the upper interior portion of the reaction vessel through a second opening located in the upper portion of the reaction vessel.

25. The process according to claim 19, wherein the directing comprises directing inert gas into a lower interior portion of the reaction vessel through a first opening located in a lower portion of the reaction vessel; and venting inert gas and evaporated deprotecting base from the upper interior portion of the reaction vessel through a second opening located in the upper portion of the reaction vessel.

26. The process according to claim 19, wherein the directing comprises directing inert gas into an upper interior portion of the reaction vessel through a first opening located in an upper portion of the reaction vessel, directing inert gas into a lower interior portion of the reaction vessel through a second opening located in a lower portion of the reaction vessel, and venting inert gas and evaporated deprotecting base from the upper interior portion of the reaction vessel through a third opening located in the upper portion of the reaction vessel.

27. The process according to claim 19, wherein the inert gas is nitrogen gas and wherein the directing comprises continuously directing the nitrogen gas through the reaction vessel.

28. The process according to claim 19, wherein the deprotecting base has a boiling point less than 107° C. and a difference between a temperature of heating the protected amino acid and the deprotecting base during the removing of the protecting group from the protected amino acid and the boiling point of the deprotecting base ranges from about 1° C. to about 50° C.

29. The process according to claim 19, wherein the deprotecting base is pyrrolidine.

30. The process according to claim 19, comprising adding deprotection solution including the deprotecting base to the reaction vessel in an amount sufficient to provide from greater than zero to about 5 vol % of the deprotecting base in the reaction vessel, based on the total volume (100 vol %) of the deprotection reaction mixture in the reaction vessel,
wherein the vol % of the deprotecting base of the deprotection solution added to the reaction vessel is greater than the vol % of the deprotecting base of the deprotecting reaction mixture after the deprotection solution is added to the reaction vessel.

31. The process according to claim 30, wherein the vol % of the deprotecting base of the deprotection solution added to the reaction vessel is not greater than about 30 vol %, based on the total volume of the deprotection solution added to the reaction vessel.

32. The process according to claim 19, wherein the protected amino acid is attached to a solid resin support having a resin substitution from 0.10 mmol/g to 0.35 mmol/g.

33. The process according to claim 32, wherein the protected amino acid is attached to a solid resin support having a resin substitution from 0.20 mmol/g to 0.33 mmol/g.

34. The process according to claim 19, wherein the process does not include washing between a deprotecting step and a coupling step of one or more deprotection-coupling cycles of the solid phase peptide synthesis process.

35. The process according to claim 19, comprising washing the interior of the reaction vessel between a deprotecting step and a coupling step of one or more deprotection-coupling cycles of the solid phase peptide synthesis process using a solvent in an amount that is 2 times or less of the bed volume of resin present in the reaction vessel.

36. The process according to claim 35, comprising washing the interior of the reaction vessel between a deprotecting step and a coupling step of one or more deprotection-coupling cycles of the solid phase peptide synthesis process using a solvent in an amount that is 1 times or less of the bed volume of resin present in the reaction vessel.

37. A process for solid phase peptide synthesis, comprising:
deprotecting a protected peptide attached to a solid resin with a resin substitution from 0.10 mmol/g to 0.35 mmol/g in a reaction vessel with a deprotecting base at a temperature that is no less than 35° C. below the boiling point of the deprotecting base and for a time of 1.5 hours or less to provide a deprotected peptide, wherein:
the deprotecting base is present in the reaction vessel in an amount great than zero to about 5 vol %, based on the total volume (100 vol %) of a deprotection reaction mixture in the reaction vessel, and
at least a portion of the deprotecting base evaporates into an upper interior portion of the reaction vessel during the deprotecting the protected peptide;
directing an inert gas through the reaction vessel to assist in the removal of evaporated deprotecting base from the interior of the reaction vessel during deprotecting the protected peptide;
washing the interior of the reaction vessel after deprotecting the protected peptide and before a successive coupling step of one or more deprotection-coupling cycles of the solid phase peptide synthesis using a solvent in an amount that is 2 times or less of a bed volume of resin present in the reaction vessel;
coupling an amino acid to the deprotected peptide to form a peptide including the deprotected peptide and the amino acid; and
repeating the deprotecting and coupling to form a peptide further comprising one or more successive amino acids.

38. A process for deprotecting a protected peptide during liquid phase peptide synthesis including deprotecting steps and coupling steps, the deprotecting process comprising:
removing a protecting group of a protected peptide in the liquid phase, wherein the C-terminus of the protected peptide is protected, in a reaction vessel with a deprotecting base at a temperature that is no less than 35° C. below the boiling point of the deprotecting base and for a time of 1.5 hours or less to provide a deprotected peptide, wherein:
the deprotecting base is present in the reaction vessel in an amount greater than zero to about 5 vol %, based on the total volume (100 vol %) of a deprotection reaction mixture in the reaction vessel, and
a majority of the deprotecting base evaporates into an upper interior portion of the reaction vessel during the removing of the protecting group of the protected peptide;
directing an inert gas through the reaction vessel to assist in the removal of evaporated deprotecting base from the interior of the reaction vessel during the removing the protecting group of the protected peptide; and
extracting residual deprotecting base from the deprotection reaction mixture after removing the protecting group and before a successive coupling step using a total volume of extraction solvent that is 2× or less than the total volume of the deprotection reaction mixture.

39. The process according to claim 38, wherein the directing comprises directing inert gas into an upper interior portion of the reaction vessel through a first opening located in an upper portion of the reaction vessel; and venting inert gas and evaporated deprotecting base from the upper interior portion of the reaction vessel through a second opening located in the upper portion of the reaction vessel.

40. A process for deprotecting a protected peptide during liquid phase peptide synthesis including deprotecting steps and coupling steps, the deprotecting process comprising:
removing a protecting group of a protected peptide in the liquid phase, wherein the C-terminus of the protected peptide is protected, in a reaction vessel with a deprotecting base at a temperature that is no less than 35° C. below the boiling point of the deprotecting base and for a time of 1.5 hours or less to provide a deprotected peptide, wherein:
the deprotecting base is present in the reaction vessel in an amount greater than zero to about 5 vol %, based on the total volume (100 vol %) of a deprotection reaction mixture in the reaction vessel, and
a majority of the deprotecting base evaporates into an upper interior portion of the reaction vessel during the removing of the protecting group of the protected peptide; and
directing inert gas into an upper interior portion of the reaction vessel through a first opening located in an upper portion of the reaction vessel; and venting inert gas and evaporated deprotecting base from the upper interior portion of the reaction vessel through a second opening located in the upper portion of the reaction vessel.

41. The process according to claim 40, further comprising extracting residual deprotecting base from the deprotection reaction mixture after removing the protecting group and before a successive coupling step using a total volume of extraction solvent that is 2× or less than the total volume of the deprotection reaction mixture.

42. A process for liquid phase peptide synthesis, comprising:
deprotecting a protected peptide in the liquid phase, wherein the C-terminus of the protected peptide is protected, in a reaction vessel to provide a deprotected peptide and wherein the deprotecting comprises:
removing a protecting group of the protected peptide with a deprotecting base at a temperature that is no less than 35° C. below the boiling point of the deprotecting base and for a time of 1.5 hours or less, wherein the deprotecting base is present in the reaction vessel in an amount great than zero to about 5 vol %, based on the total volume (100 vol %) of a deprotection reaction mixture in the reaction vessel, and wherein a majority of the deprotecting base evaporates into an upper interior portion of the reaction vessel during the removing of the protecting group; and
directing an inert gas through the reaction vessel to remove evaporated deprotecting base from the interior of the reaction vessel during the removing of the protecting group;
extracting residual deprotecting base from the deprotection reaction mixture after removing the protecting group and before a successive coupling step using a total volume of extraction solvent that is 2× or less than the total volume of the deprotection reaction mixture;
coupling an amino acid to the deprotected peptide to form a peptide from the deprotected peptide and the amino acid; and
repeating the deprotecting, extracting, and coupling to form a peptide further comprising one or more successive amino acids.

43. The process according to claim 42, wherein the directing comprises directing inert gas into an upper interior portion of the reaction vessel through a first opening located in an upper portion of the reaction vessel; and venting inert gas and evaporated deprotecting base from the upper interior portion of the reaction vessel through a second opening located in the upper portion of the reaction vessel.

44. A process for liquid phase peptide synthesis, comprising:
deprotecting a protected peptide in the liquid phase, wherein the C-terminus of the protected peptide is protected, in a reaction vessel to provide a deprotected peptide and wherein the deprotecting comprises:
removing a protecting group of the protected peptide with a deprotecting base at a temperature that is no less than 35° C. below the boiling point of the deprotecting base and for a time of 1.5 hours or less, wherein the deprotecting base is present in the reaction vessel in an amount great than zero to about 5 vol %, based on the total volume (100 vol %) of a deprotection reaction mixture in the reaction vessel, and wherein a majority of the deprotecting base evaporates into an upper interior portion of the reaction vessel during the removing of the protecting group; and
directing inert gas into an upper interior portion of the reaction vessel through a first opening located in an upper portion of the reaction vessel; and venting inert gas and evaporated deprotecting base from the upper interior portion of the reaction vessel through a second opening located in the upper portion of the reaction vessel to remove evaporated deprotecting base from the reaction vessel during the removing of the protecting group;
coupling an amino acid to the deprotected peptide to form a peptide from the deprotected peptide and the amino acid; and
repeating the deprotecting and coupling to form a peptide further comprising one or more successive amino acids.

45. The process according to claim 44, further comprising extracting residual deprotecting base from the deprotection reaction mixture after removing the protecting group and before a successive coupling step using a total volume of extraction solvent that is 2× or less than the total volume of the deprotection reaction mixture.
